(19) United States
(12) Patent Application Publication
(10) Pub. No.: US 2005/0239751 A1
(43) Pub. Date: Oct. 27, 2005

(54) Heterocyclic anti-viral compounds comprising metabolizable moieties and their uses

(75) Inventors: Rajinder Singh, Belmont, CA (US); Dane Goff, Redwood City, CA (US); Rao S. S. Kolluri, Foster City, CA (US); Ihab S. Darwish, San Mateo, CA (US); John J. Partridge, Chapel Hill, NC (US); Robin Cooper, St. George Island, FL (US); Henry H. Lu, Foster City, CA (US); Gary Park, Moss Beach, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Rigel Pharmaceuticals, Inc., South San Francisco, CA (US)

(21) Appl. No.: 11/090,823

(22) Filed: Mar. 25, 2005

Related U.S. Application Data
(60) Provisional application No. 60/556,625, filed on Mar. 26, 2004.
(60) Provisional application No. 60/582,903, filed on Jun. 24, 2004.

(51) Int. Cl. A61K 31/675; A61K 31/497; A61K 31/496; A61K 31/4439

(52) U.S. Cl. 514/79; 514/253.1; 514/337; 546/268.4; 548/112; 514/362; 514/364; 544/360

(57) ABSTRACT

The present invention relates to substituted prodrug and compositions thereof useful for treating or preventing Hepatitis C virus (HCV) infections. In particular, the present invention relates to prodrugs of substituted diphenyl-, diheteroaryl- and mixed phenyl heteroaryl substituted five-membered heterocycle compounds, compositions comprising the compounds and the use of such compounds and compositions to inhibit HCV replication and/or proliferation as a therapeutic approach towards the treatment and/or prevention of HCV infections in humans and animals.

This application claims priority to U.S. Provisional application No. 60/556,625 filed Mar. 26, 2004 and to U.S. Provisional application No. 60/582,903 filed Jun. 24, 2004.

### FIELD OF INVENTION

The present invention relates to substituted prodrug and compositions thereof useful for treating or preventing Hepatitis C virus (HCV) infections. In particular, the present invention relates to prodrugs of substituted diphenyl-, diheteroaryl- and mixed phenyl heteroaryl substituted five-membered heterocycle compounds, compositions comprising the compounds and the use of such compounds and compositions to inhibit HCV replication and/or proliferation as a therapeutic approach towards the treatment and/or prevention of HCV infections in humans and animals.

### BACKGROUND OF THE INVENTION

Hepatitis C virus (HCV) infection is a global human health problem with approximately 150,000 new reported cases each year in the United States alone. HCV is a single stranded RNA virus, which is the etiological agent identified in most cases of non-A, non-B post-transfusion and post-transplant hepatitis and is a common cause of acute sporadic hepatitis (Choo et al., Science 244:359, 1989; Kuo et al., Science 244:362, 1989; and Alter et al., in Current Perspective in Hepatology, p. 83,1989).

It is estimated that more than 50% of patients infected with HCV become chronically infected and 20% of those develop cirrhosis of the liver within 20 years (Davis et al., New Engl. J. Med. 321:1501, 1989; Alter et al., in Current Perspective in Hepatology, p. 83, 1989; Alter et al., New Engl. J. Med. 327:1899, 1992; and Dienstag Gastroenterology 85:430, 1983). Moreover, the only therapy available for treatment of HCV infection is interferon-α (INTRON® A, PEG-INTRON®A, Schering-Plough; ROFERON-A®, PEGASys®, Roche). Most patients are unresponsive, however, and among the responders, there is a high recurrence rate within 6-12 months after cessation of treatment (Liang et al., J. Med. Virol. 40:69, 1993). Ribavirin, a guanosine analog with broad spectrum activity against many RNA and DNA viruses, has been shown in clinical trials to be effective against chronic HCV infection when used in combination with interferon- [symbol] (see, e.g., Poynard et al., Lancet 352:1426-1432, 1998; Reichard et al., Lancet 351:83-87, 1998), and this combination therapy has been recently approved (REBETRON, Schering-Plough; see also Fried et al., 2002, N. Engl. J. Med. 347:975-982). However, the response rate is still at or below 50%. Therefore, additional compounds for treatment and prevention of HCV infection are needed.

### SUMMARY OF THE INVENTION

The invention provides compounds, compositions and methods comprising substituted heterocyclic prodrugs that are potent inhibitors of Hepatitis C virus (“HCV”) replication and/or proliferation.

In a second aspect, the invention provides methods of making the prodrugs of formula (I). Specific embodiments of the methods are illustrated in FIGS. 1-10. In one embodiment, the method for synthesizing compounds according to structural formula (I) comprises acetylating a compound according to structural formula (Ill) with a dihaloacetyl halide.

In a third aspect, the invention provides prodrug compositions. The compositions generally comprise prodrugs of the invention, or salts, hydrates, solvates, or N-oxides thereof and a suitable excipient, carrier or diluent. The composition may be formulated for veterinary uses or for use in humans.

The prodrugs of the invention, the resultant active drug transformed from the prodrug, or the active compound produced after metabolism are potent inhibitors of HCV replication and/or proliferation. Accordingly, in still a fourth aspect, the invention provides methods of inhibiting HCV replication and/or proliferation, comprising contacting a Hepatitis C virion with an amount of a prodrug or composition of the invention effective to inhibit its replication or proliferation. The methods may be practiced either in vitro or in vivo, and may be used as a therapeutic approach towards the treatment and/or prevention of HCV infections.

In a fifth aspect, the invention provides methods of treating, preventing, and/or inhibiting HCV infections. The methods generally involve administering to a subject that has an HCV infection or that is at risk of developing an HCV infection with an amount of a prodrug or composition of the invention effective to treat or prevent the HCV infection. The method may be practiced in animals in veterinary contexts or in humans.

### BRIEF DESCRIPTION OF THE FIGURES

FIG. 1 shows a general synthetic scheme for phosphonate containing compounds of the invention.

FIG. 2 shows an alternative general synthetic scheme for phosphonate containing compounds compounds of the invention.

FIG. 3 shows synthetic schemes for two phosphonate containing compounds of the invention.

FIG. 4 shows a general synthetic scheme for ester containing compounds of the invention.

FIG. 5 shows an alternative general synthetic scheme for ester containing compounds of the invention.

FIG. 6 shows a synthetic scheme for an ester containing compound of the invention.

FIG. 7 shows a synthetic scheme for an ester containing compound of the invention.

FIGS. 8a and 8b show a synthetic scheme for a dioxolenone containing compound of the invention.

FIGS. 9a and 9b show a synthetic scheme for a dioxolenone containing compound of the invention.

FIG. 10 shows a synthetic scheme for a dioxolenone containing compound of the invention.

FIG. 11 shows a synthetic scheme for preparing compound 311a, an alkyl acetamide.

FIG. 12 shows a general synthetic scheme for preparing alkyl acetamide containing prodrugs.

FIG. 13 shows a synthetic scheme for preparing compound 409a, an alkyl acetamide.

FIG. 14 shows an general synthetic scheme for preparing alkylacetamide containing prodrugs.

FIG. 15 shows a synthetic scheme for preparing compound 605a, an alkyl acetamide.

FIG. 16 shows a general synthetic scheme for preparing alkyl acetamide containing prodrugs.

FIGS. 17A and 17B show the metabolism of the active parent compound in human microsomes.

FIGS. 18A and 18B show the hydrolysis of the active parent compound in rat.

FIG. 19 shows the hydrolysis of the active parent compound in cynomolgus monkey.

FIGS. 20A and 20B show excretion of the active parent compound in rat.

FIGS. 21A, 21b and 21C show the absorption of the active parent compound in cynomolgus monkey.

FIG. 22 shows the excretion of the active parent compound in rat.

FIG. 23 shows a synthetic scheme for preparing compound 1045.

FIG. 24 shows a synthetic scheme for preparing compound 1046.

FIG. 25 shows a synthetic scheme for preparing compound 1047.

FIG. 26 shows a synthetic scheme for preparing compound 1048.

FIG. 27 shows a synthetic scheme for preparing compound 1028.

FIG. 28 shows a synthetic scheme for preparing compound 1027.

FIG. 29 shows a synthetic scheme for preparing compound 1010.

FIG. 30 shows a synthetic scheme for preparing compound 1014.

FIG. 31 shows a synthetic scheme for preparing compound 1024.

FIG. 32 shows a synthetic scheme for preparing compound 1034.

FIG. 33 shows a synthetic scheme for preparing compound 1037.

FIG. 34 shows a synthetic scheme for preparing compound 1042.

FIG. 35 shows a synthetic scheme for preparing compound 1044.

### DETAILED DESCRIPTION OF THE INVENTION

The invention provides compounds, compositions and methods comprising substituted heterocyclic prodrugs that are potent inhibitors of Hepatitis C virus (“HCV”) replication and/or proliferation.

In the first aspect, the invention provides a compound of the formula
A—B—C-(C(O))ₛ—N(R¹¹)—C(O)—CX₂—R¹² (I)
or a pharmaceutically acceptable salt, hydrate, solvate or N-oxide thereof, wherein
A is a phenyl or six-membered heteroaryl ring having from one to five of the same or different R²⁰ substituents, provided that at least one of the substituents is positioned at the ortho position;
B is a saturated, unsaturated, or aromatic heteroatomic ring having from one to three annular heteroatoms selected from N, O, and S, where the A and C moieties are attached to non-adjacent ring atoms of B, provided that when the B includes more than one annular oxygen atom, the oxygen atoms are not adjacent;
C is a phenyl or a heteroaryl ring; in certain embodiments, wherein when C is phenyl, it is substituted relative to the B moiety at the meta position with the —C(O))ᵣN(R¹¹)—C(O)—CX₂—R¹², or when C is a heteroaryl group, the B moiety and the —C(O))ᵣN(R¹¹)—C(O)—CX₂—R¹² moiety are positioned on C with only one ring atom of C between them;
s is 0 or 1;
R¹¹ is selected from the group consisting of hydrogen, lower alkyl, -(CHR¹⁰)ₙ—J—G, or a group of the formula 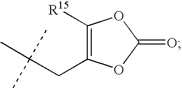
each X is independently H or halo, provided both X are not H;
R¹² is selected from the group consisting of hydrogen, —M(O)-alkyl, —C(O)OR¹⁶, —C(O)R¹⁷ and —P(O)(OR¹⁸)OR¹⁹;
R¹⁵ is lower alkyl, arylalkyl, aryl, substituted cycloheteroalkyl, cycloheteroalkyl, substituted cycloalkyl, cycloalkyl, —C(O)OR¹⁸ or —CH₂—OR³⁰;
R³⁰ is hydrogen, lower alkyl or a sugar moiety;
R¹⁶ is selected from the group consisting of aryl-C₁-C₆ alkyl, aryl, substituted cycloheteroalkyl, cycloheteroalkyl, substituted cycloalkyl, cycloalkyl, —C(O)OR¹⁶ or —CH₂—OR³⁰, (C₁₋₁₅) alkyl and (C₇₋₁₅) arylalkyl;
R¹⁷ is selected from the group consisting of lower alkyl, —N(R^{c})₂, N-morpholino, N-piperazino and N-pyrrolidino;
each R^{c} is independently R^{b} or alternatively, the both R^{c} taken together with the nitrogen atom to which they are bonded form a 5-, 6- or 7-membered cycloheteroalkyl which optionally includes from 1 to 4 of the same or different additional heteroatoms selected from the group consisting of O, N and S;
each R^{b} is independently hydrogen or R^{a};
R^{a} is selected from the group consisting of alkyl, hydroxyalkyl, cycloalkyl, heteroalkyl, C₀-C₆ alkyl-cycloheteroalkyl, substituted C₀-C₆ alkyl-cycloheteroalkyl, aryl, arylalkyl, heteroaryl and heteroarylalkyl;
R¹⁸ is H, lower alkyl, aryl or arylalkyl;
R¹⁹ is H, lower alkyl, aryl or arylalkyl;
n is 0, 1, 2, 3 or 4;
each R¹⁰ is independently hydrogen or lower alkyl;
J is selected from the group consisting of -(CH₂)₁₋₃-, —O—, —O—(CH₂)₁₋₃-, —CH(OH)-, —C(═O)-, —S(O)ₘ-, —C(═NR²³)-, —C(═NOR²⁹)-, —C(N—N-(R²⁵)₂)-, —C(N—NR²⁷C(═O)N-(R²⁷)₂)- and —C(Z—R²⁸)₂-;
m is 0, 1, or 2;
R²³ is selected from the group consisting of hydrogen, lower alkyl, aryl, substituted aryl, arylalkyl and substituted arylalkyl;
R²⁹ is selected from the group consisting of hydrogen, lower alkyl, aryl, substituted aryl, arylalkyl and substituted arylalkyl;
each R²⁵ is independently selected from the group consisting of hydrogen, lower alkyl, aryl, substituted aryl, arylalkyl and substituted arylalkyl;
each R²⁷ is independently selected from the group consisting of hydrogen, lower alkyl, aryl, substituted aryl, arylalkyl and substituted arylalkyl;
each Z is independently —O— or —S—;
each R²⁸ is independently selected from the group consisting of lower alkyl, aryl, substituted aryl, arylalkyl and substituted arylalkyl or alternatively, the two R¹⁸'s taken together with the heteroatoms to which they are bonded form a 5, 6 or 7 membered cycloheteroalkyl;
G is selected from the group consisting of aryl, substituted aryl, arylalkyl, substituted arylalkyl, heteroaryl, substituted heteroaryl, cycloheteroalkyl and substituted cycloheteroalkyl; and
each R²⁰ is, independently of the other, selected from the group consisting of —OH, —SH, —CN, —C(O)H, —NO₂, halo, fluoro, chloro, bromo, iodo, lower alkyl, substituted lower alkyl, lower heteroalkyl, substituted lower heteroalkyl, cycloalkyl, substituted cycloalkyl, cycloheteroalkyl, substituted cycloheteroalkyl, lower haloalkyl, monohalomethyl, dihalomethyl, trihalomethyl, trifluoromethyl, lower alkylthio, substituted lower alkylthio, lower alkoxy, substituted lower alkoxy, methoxy, substituted methoxy, lower heteroalkoxy, substituted lower heteroalkoxy, cycloalkoxy, substituted cycloalkoxy, cycloheteroalkoxy, substituted cycloheteroalkoxy, lower haloalkoxy, monohalomethoxy, dihalomethoxy, trihalomethoxy, trifluoromethoxy, amino, lower di- or monoalkylamino, substituted lower di- or monoalkylamino, aryl, substituted aryl, aryloxy, substituted aryloxy, phenoxy, substituted phenoxy, arylalkyl, substituted arylalkyl, arylalkyloxy, substituted arylalkyloxy, benzyl, benzyloxy, heteroaryl, substituted heteroaryl, heteroaryloxy, substituted heteroaryloxy, heteroarylalkyl, substituted heteroarylalkyl, heteroarylalkyloxy, substituted heteroarylalkyloxy, carboxyl, lower alkoxycarbonyl, substituted lower alkoxycarbonyl, aryloxycarbonyl, substituted aryloxycarbonyl, arylalkyloxycarbonyl, substituted arylalkyloxycarbonyl, carbamate, substituted carbamate, carbamoyl, substituted carbamoyl, thiocarbamoyl, substituted thiocarbamoyl, ureas, substituted ureas, thioureas, substituted thioureas, sulfamoyl, substituted sulfamoyl and a group of the formula —LR¹⁴, where “L” is a linker and R¹⁴ is cycloalkyl, substituted cycloalkyl, cycloheteroalkyl or substituted cycloheteroalkyl;
provided that when R¹¹ is hydrogen or lower alkyl, then R¹² is not hydrogen.

In Embodiment A1 according to formula (1), s is 1, R¹² is —H, and R¹¹ is a group of the formula 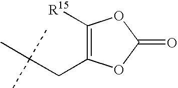
wherein R¹⁵ is CH₂—OR³⁰. In some embodiments, R³⁰ is a sugar moiety.

In Embodiment A according to formula (I), s is O, R¹² is —H, and R¹¹ is a group of the formula 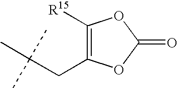
wherein R¹⁵ is lower alkyl, arylalkyl, aryl, cycloheteroalkyl, cycloalkyl, or —CH₂—OR³⁰. In some embodiments, R¹⁵ is piperidyl, pyrrolidinyl, t-butyl, benzyl, cyclobutyl or propyl. In some embodiments R¹⁵ is —CH₂—OR³⁰. In some embodiments, R³⁰ is a sugar moiety.

In Embodiment B according to formula (I), s is O, R¹¹ is selected from the group consisting of hydrogen or -(CHR¹⁰)ₙ—J—G, wherein n is 0, 1 or 2, J is -(CH₂)₁₋₃-, —C(O)-, —O— or —O—(CH₂)₁₋₃-, and G is substituted aryl, cycloheteroalkyl, substituted cycloheteroalkyl or heteroaryl. In some embodiments, R¹¹ is hydrogen. In some embodiments G is pyrrolidinyl, morpholinyl or imidazolyl. In some embodiments G is phenyl substituted with methoxy, -chloro, fluoro, CH₂—P(O)(OR^{b})(OR^{b}), —O—P(O)(OR^{b})(OR^{b}), methyl, —O—C(O)NH—R^{b}, —NR^{b}C(O)OR^{b}, ethyl-piperazinyl, piperazinyl, t-butyl-O—C—(O)-piperazinyl, —O—(CH₂)₀₋₄—R^{b}, or —C(O)OR^{b}, wherein R^{b} is —H, propyl, t-butyl, ethyl, or morpholinyl.

In Embodiment C according to formula (I), s is O, each X is chloro, and R¹² is selected from the group consisting of hydrogen, —C(O)OR¹⁵, —C(O)R¹⁷ and —P(O)(OR¹⁸)OR¹⁹; R¹⁵ is lower alkyl, arylalkyl, substituted cycloalkyl, or cycloalkyl; R¹⁷ is selected from the group consisting of lower alkyl, —N(R^{c})₂, or N-morpholino; each R^{c} is independently hydrogen, alkyl, hydroxyalkyl, C₀-C₆ alkyl-cycloheteroalkyl, or heteroarylalkyl; and R¹⁸ and R¹⁹ are independently H or lower alkyl. In some embodiments, R¹² is —P(O)(OR¹⁸)OR¹⁹ wherein R¹⁸ and R¹⁹ are both —H, ethyl or propyl. In some embodiments R¹² is —C(O)OR¹⁵. In some embodiments, R¹⁵ is adamantane methyl, propyl, —CH₂-phenyl, t-butyl, cyclohexyl, or cyclohexyl substituted with methyl, propyl, or pentyl. In some embodiments R¹² is —C(O)R¹⁷. In some embodiments, R¹⁷ is methyl, N-morpholino, or —N(R^{c})₂. In some embodiments, each RC is independently hydrogen, -(CH₂)₃-morpholinyl, —CH₂-pyridinyl or -(CH₂)₂—OH.

In Embodiment D according to formula (I), s is O and A is phenyl substituted with at least two R²⁰ groups selected from the group consisting of halo, lower alkoxy, carboxyl, lower haloalkyl, cycloalkyl, lower alkoxycarbonyl and —L—R¹⁴, wherein “L” is a —O— and R¹⁴ is cycloheteroalkyl or substituted cycloheteroalkyl. In some embodiments, A represents a phenyl ring substituted at the 2- and 6-positions with the same or different R²⁰ substituent. In some embodiments one R²⁰ is halo and the other R²⁰ is lower alkoxy, lower haloalkyl or cycloalkyl. In some embodiments, one R²⁰ is chloro and the other R²⁰ is methoxy, —CF₃, or cyclopropyl. In some embodiments one R²⁰ is halo and the other R²⁰ is carboxyl or lower alkoxycarbonyl. In some embodiments, one R²⁰ is chloro and the other R²⁰ is methoxycarbonyl. In some embodiments one R²⁰ is halo and the other R²⁰ is —O—R¹⁴, wherein R¹⁴ is morpholinyl or morpholinyl substituted with —C(O)—O—t-butyl or —C(O)—CH₃.

In Embodiment E according to formula (I), s is O and A represents a pyrid-2-yl ring substituted at the 3-position with an R²⁰ substituent, a pyrid-3-yl ring substituted at the 2- and 4-positions with the same or different R²⁰ substituents or a pyrid4-yl ring substituted at the 3- and 5-positions with the same or different R²⁰ substituents. In some embodiments, each R²⁰ is independently selected from the group consisting of halo, lower dialkylamino and —L—R¹⁴, wherein “L” is a —O— and R¹⁴ is cycloheteroalkyl. In some embodiments each R²⁰ is independently selected from the group consisting of chloro, —N(CH₃)₂, and —O—R¹⁴, wherein R¹⁴ is morpholinyl.

In Embodiment F according to formula (I), s is O and C represents a phenyl ring, a pyrid-2-yl ring or a pyrid-3-yl ring.

In Embodiment G according to formula (I), s is O and B represents a isoxazolyl, pyrazolyl, oxadiazolyl or triazolyl ring.

In Embodiment H according to formula (I), s is O and B is 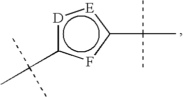
wherein D, E and F are each, independently of one another, selected from N, O and CH, provided that at least two of D, E and F are other than CH and D and E are not both simultaneously O.

In Embodiment I according to formula (I), s is O and each X is —Cl. In many examples of Embodiment I, A is phenyl and C is phenyl or pyridyl. More particularly, in some examples, the compound is according to one of the following nine formula:

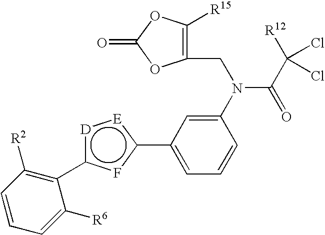

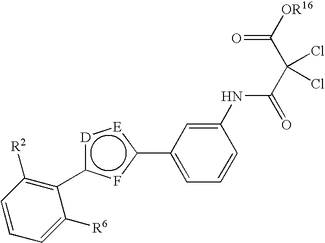

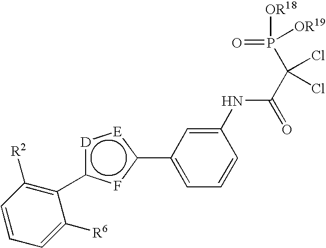

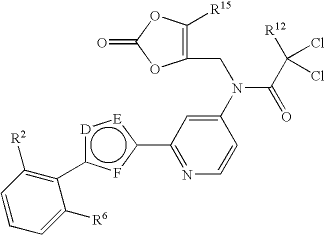

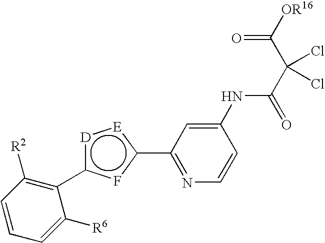

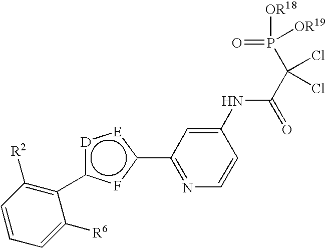

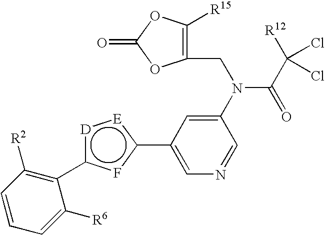

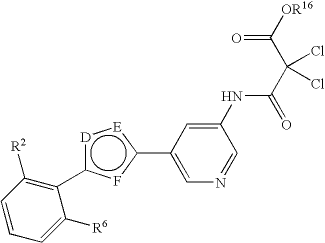

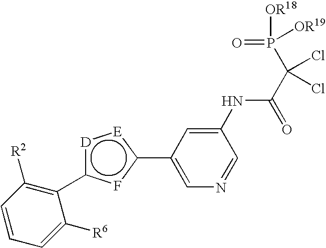

wherein R² and R⁶ are R²⁰ and each, independently of one another, is selected from the group consisting of —OH, —NO₂, halo, fluoro, chloro, bromo, iodo, lower alkyl, methyl, lower heteroalkyl, (C₃-C₆) cycloalkyl, 5- or 6-membered cycloheteroalkyl, N-morpholinyl, N-methyl-N-piperazinyl, N-piperadinyl, substituted N-piperadinyl, 44N-piperadinyl)-N-piperadinyl, 4-amino-N-piperadinyl, lower alkoxy, methoxy, ethoxy, lower alkylthio, methylthio, lower haloalkyl, monohalomethyl, dihalomethyl, trihalomethyl, trifluoromethyl, lower haloalkyloxy, monohalomethoxy, dihalomethoxy, trihalomethoxy, trifluoromethoxy, aryl, phenyl, arylalkyl, benzyl, aryloxy, phenoxy, arylalkyloxy, benzyloxy, 5- or 6-membered heteroaryl, lower alkyloxycarbonyl, sulfamoyl and —L—R¹⁴, where L is -(CH₂)₁₋₃- or —O—(CH₂)₁₋₃- and R¹⁴ is a 5- or 6-membered cycloheteroalkyl or N-morpholinyl. In some embodiments, R² and R⁶ are each, independently of one another, selected from the group consisting of chloro, bromo, iodo and fluoro. In some embodiments, D is O, E is N and F is CH, or D is N, E is O and F is CH. In some embodiments, R¹² is —H and R¹⁵ is lower alkyl, arylalkyl, aryl, cycloheteroalkyl, cycloalkyl, or a sugar moiety. In some embodiments, R¹⁵ is piperidyl, pyrrolidinyl, t-butyl, benzyl, cyclobutyl or propyl. In some embodiments, R¹⁶ is lower alkyl or cycloalkyl. Preferably, R¹⁶ is t-butyl or adamantane. In some embodiments, R¹⁸ is —H or lower alkyl and R¹⁹ is H or lower alkyl. In some embodiments, R¹⁸ and R¹⁹ are both —H, ethyl or propyl.

In Embodiment J according to formula (I), s is O and G is aryl or substituted aryl. In some embodiments, G is phenyl substituted with one or more groups selected from hydrogen, —F, —Cl, —OMe, —CO₂H, —CO₂t—Bu, —CH₂CO₂Et, methyl —OC(O)CH₃, —OC(O)CH₂N(CH₃)₂, —OC(O)CH₂N(CH₃)Boc, —OC(O)CH₂NH(CH₃), or

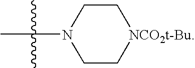

In Embodiment K according to formula (I), s is O and G is substituted arylalkyl, heteroaryl, cycloheteroalkyl or substituted cycloheteroalkyl. In some examples, G is

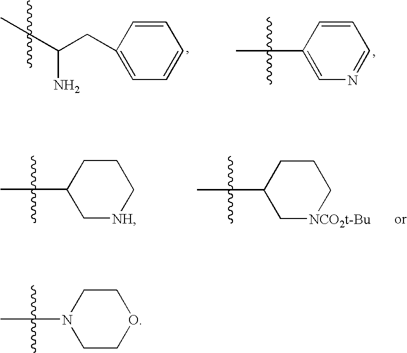

In Embodiment L according to formula (I) are compounds that, when administered to a cell comprising a hepatitis C virion, inhibits HCV replication and/or proliferation, and have an IC₅₀ of 10 μM or less, as measured in an in vitro assay.

In the third aspect, the invention provides a composition comprising a pharmaceutically acceptable vehicle and a compound according to the first aspect and Embodiments A1-L.

In the fourth aspect, the invention provides a method of inhibiting replication and/or proliferation of a hepatitis C (“HC”) virion, comprising the step of contacting an HC virion with an amount of a compound according to according to the first aspect and Embodiments A-L effective to inhibit replication and/or proliferation of the HC virion. In some embodiments, the method is practiced in vitro or in vivo. In some embodiments, the method of treating or preventing an HCV infection, comprising the step of administering to a subject an amount of a compound according to according to the first aspect and Embodiments A-L effective to treat or prevent an HCV infection. In some embodiments, the subject is a human. In some embodiments, the compound is administered in an amount of about 0.1 mg/kg/day to 200 mg/kg/day. In some embodiments, the compound is administered in an amount of about 10 mg/kg/day to 100 mg/kg/day. In some embodiments compound is administered orally, intravenously or subcutaneously. In some embodiments, the method is practiced therapeutically in a subject having an HCV infection, or practiced prophylactically in a subject at risk of developing an HCV infection.

In one embodiment according to the second aspect, the invention provides an intermediate compound useful for synthesizing substituted heterocycle compounds, said intermediate compound having the formula (IV): 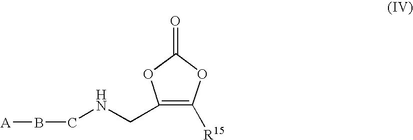
wherein A, B, C and R¹⁵ are as defined in the first aspect and Embodiments A-L. Like the compounds of structural formulae (I) and (III), the double bonding pattern of the “B” ring will depend upon the identities of the heteroatoms therein. The invention also comprises a method of synthesizing a heterocycle compound of formula (XI): 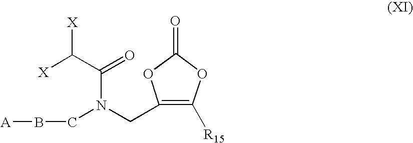
wherein A, B, C, X and R¹⁵ are as defined in the first aspect and Embodiments A-L, comprising dihaloacetylating a compound of the formula (IV), thereby yielding a compound according to formula (XI).

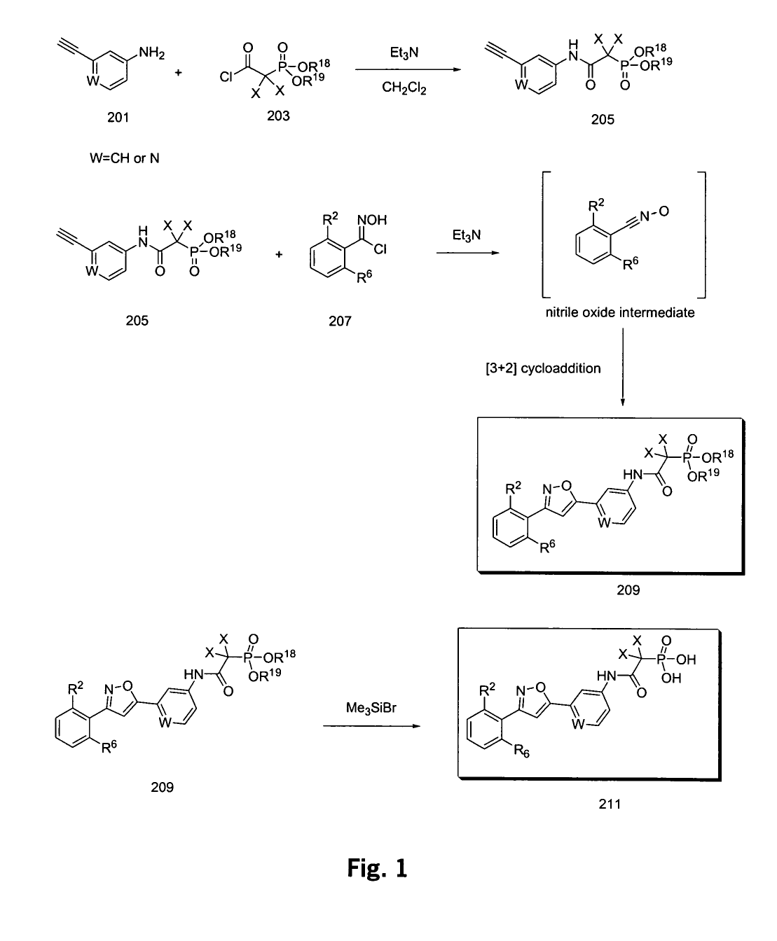

In Embodiment M according to the second aspect, the invention provides starting and intermediate compounds useful for synthesizing the compounds of the invention. Representative starting and intermediate compounds useful for synthesizing prodrugs of the invention include compounds 201, 203, 205, 207, 209, 301, 401, 403, 405, 501, 503, 603, 605, 801, 803, 805, 807, 903, 905, 1003, and 1005 as depicted in FIGS. 1-10.

Prodrugs having the structural formulae (I) and (II) can be prepared from heterocyclic compounds described in U.S. Ser. Nos. 10/286,017, filed Nov. 1, 2002, 60/467,650, filed May 2, 2003, 60/467,811, filed May 2, 2003, 10/440,349, filed May 15, 2003, 10/646,348, filed August 22, 2003, the contents of which are incorporated herein in their entirety.

In Embodiment N according to the third aspect, some starting materials used for making compounds of the invention are according to structural formula (III)
A—B—C−NH₂ (III)
wherein A, B and C are as previously defined. Compounds having structural formula (III) are treated with, for example, either a 2, 2-dihalo-24dialkoxyphosphonyl)acetyl halide or a 24alkyloxycarbonyl)2,2-dichloroacetyl chloride to form the corresponding acetamides. Alternatively, compounds having structural formula (Ill) are treated with 4-bromomethyl-5-alkyl-1,3-dioxol-2-one to provide corresponding N-[(5-alkyl-1,3-dioxolene-2-one-4-yl)methylene]anilines. Optionally, the acetamide or aniline can be further treated with a dihaloacetyl halide to form prodrugs to structural formulae (I).

In Embodiment N1, according to the third aspect, prodrug moieties of the invention may be pre-incorporated into, for example, an acetamide intermediate containing ring C. In one example, such intermediates contain an alkynyl group that is used in conjunction with another intermediate containing ring A. When combined, for example in a [3+2] cycloaddition reaction to form ring B, prodrug compounds of the invention are formed.

In Embodiment O according to the first aspect, prodrugs having formulae (V) through (X) are useful to treat HCV infection. These include 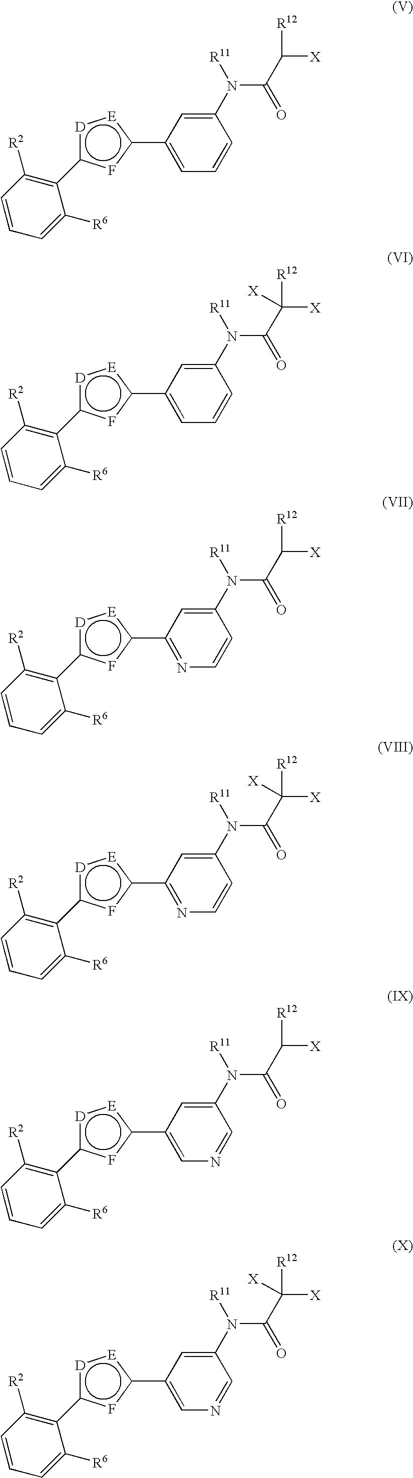
or their pharmaceutically acceptable salts, hydrates, solvates and N-oxides thereof, wherein:
D, E and F are each, independently of one another, selected from N, O and CH, provided that at least two of D, E and F are other than CH and D and E are not both simultaneously O;
R² and R⁶ are R²⁰ and each, independently of one another, is selected from the group consisting of —OH, —NO₂, halo, fluoro, chloro, bromo, iodo, lower alkyl, methyl, lower heteroalkyl, (C₃-C₆) cycloalkyl, 5- or 6-membered cycloheteroalkyl, N-morpholinyl, N-methyl-N-piperazinyl, N-piperadinyl, substituted N-piperadinyl, 44N-piperadinylkN-piperadinyl, 4-amino-N-piperadinyl, lower alkoxy, methoxy, ethoxy, lower alkylthio, methylthio, lower haloalkyl, monohalomethyl, dihalomethyl, trihalomethyl, trifluoromethyl, lower haloalkyloxy, monohalomethoxy, dihalomethoxy, trihalomethoxy, trifluoromethoxy, aryl, phenyl, arylalkyl, benzyl, aryloxy, phenoxy, arylalkyloxy, benzyloxy, 5- or 6-membered heteroaryl, lower alkyloxycarbonyl, sulfamoyl and —L—R¹⁴, where L is -(CH₂)₁₋₃- or —O—(CH₂)₁₋₃- and R¹⁴ is a 5- or 6-membered cycloheteroalkyl or N-morpholinyl; and
each individual X, R¹¹ and R¹² are as previously defined. In some embodiments, D is O, E is N and F is CH, or D is N, E is O and F is CH.

In Embodiment P, the invention provides compounds of the formula (XII) 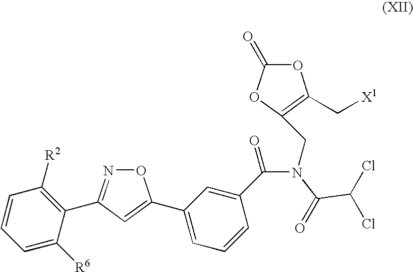
wherein R² and R⁶ are R²⁰ and each, independently of one another, is selected from the group consisting of —OH, —NO₂, halo, fluoro, chloro, bromo, iodo, lower alkyl, methyl, lower heteroalkyl, (C₃-C₆) cycloalkyl, 5- or 6-membered cycloheteroalkyl, N-morpholinyl, N-methyl-N-piperazinyl, N-piperadinyl, substituted N-piperadinyl, 44N-piperadinylkN-piperadinyl, 4-amino-N-piperadinyl, lower alkoxy, methoxy, ethoxy, lower alkylthio, methylthio, lower haloalkyl, monohalomethyl, dihalomethyl, trihalomethyl, trifluoromethyl, lower haloalkyloxy, monohalomethoxy, dihalomethoxy, trihalomethoxy, trifluoromethoxy, aryl, phenyl, arylalkyl, benzyl, aryloxy, phenoxy, arylalkyloxy, benzyloxy, 5- or 6-membered heteroaryl, lower alkyloxycarbonyl, sulfamoyl and —L—R¹⁴, where L is -(CH₂)₁₋₃- or —O—(CH₂)₁₋₃- and R¹⁴ is a 5- or 6-membered cycloheteroalkyl or N-morpholinyl; X¹ is hydroxyl, C₁-C₆ alkoxy, C₁-C₆ hydroxyalkoxy, C₁-C₆ alkoxy-C₁-C₆ alkoxy, poly-C₁-C₆ alkoxy or a sugar moiety, or X¹ is any substituted or unsubstituted tetrahydropyran or 6-(hydroxymethyl)-tetrahydro-2H-pyran-2,4,5-triol, hexahydropyrano[2,3-d][1, 3]dioxine-5, 7-diol or 5, 7-dihydroxy-hexahydropyrano[2, 3-d][1, 3]dioxin-2-one, or 6-hydroxy-44hydroxymethyl)-tetrahydro-3aH-[1 ,3]dioxolo[4, 5-c]pyran-2-one. In some embodiments, X¹ is one of the following substructures: 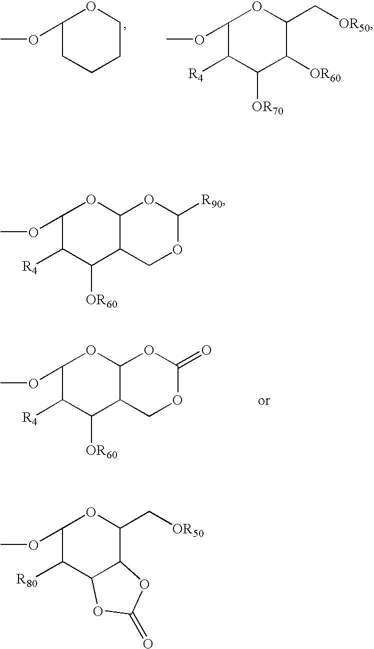
wherein R₅₀, R₆₀ and R₇₀ are independently C₁-C₆ alkyl, C₀-C₆ alkylaryl, acyl, C₀-C₆ alkyl-C(═O)-aryl, C₀-C₆ alkyl-C(═O)-C₁-C₆ alkyl, acetyl, benzoyl, benzyl, tert-butyldimethylsilyl, tert-butyldiphenylsilyl, hydrogen, or —Si(R₁₀₀)₃, wherein each R₁₀₀ is the same or different and is C₁-C₆ alkyl or C₀-C₆ alkylaryl; R₈₀ is C₁-C₆ alkyl, methyl or hydrogen; and R₉₀ is —H, or any alkyl chain or aryl group or substituted aryl group. For example,
R₅₀, R₆₀ and R₇₀ are independently 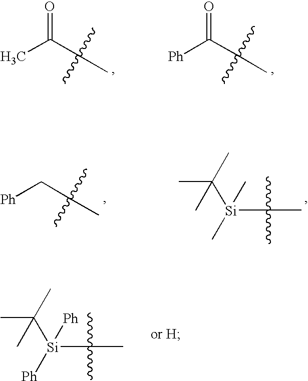
R₈₀ is —H or —CH₃; and
R₉₀ is C₁-C₆ alkyl, aryl or substituted aryl. For example, X¹ may be hydroxyethoxy, methoxymethoxy or polyethylene glycol. In some embodiments R² and R⁶ are each, independently of one another, selected from the group consisting of chloro, bromo, iodo and fluoro.

### Definitions

As used herein, the following terms are intended to have the following meanings: A “sugar moiety” is any substituted or unsubstituted saccharide having the general composition (CH₂O)ₙ and simple derivatives thereof. For examples, a sugar moiety is any naturally occurring monosaccharide including, but not limited to, (CH₂O)₆ or C₆H₁₂O₆ molecules such as aldoses, for example, D-glucose and ketoses, for example, D-fructose. A sugar moiety also includes naturally occurring disaccharides that are formed chemically or enzymatically from two monosaccharides 2((CH₂O)₆) minus an H₂O molecule to give C₁₂H₂₂O₁₁. For example, a sugar moiety includes molecules like lactose (milk sugar), maltose (malt sugar) and sucrose (cane sugar). A sugar moiety also comprises sugars in cyclic form, for example, glucose, α-D-glucose and/or its anomeric form β-glucose, fructose, the five-membered ring furanose form of fructose and the six-membered ring pyranose form of fructose. Additionally, a sugar moiety includes a tetrahydropyranyloxy group substituted with at least one hydroxyl or alkoxyl. For example, a sugar moiety any substituted or unsubstituted tetrahydropyran or
6-(hydroxymethyl~tetrahydro-2H-pyran-2,4,5-triol, hexahydropyrano[2, 3-d][1 ,3]dioxine-5, 7-diol or 5,7-dihydroxy-hexahydropyrano[2,3-d][1,3]dioxin-2-one, 6-hydroxy-4-(hydroxymethyl)-tetrahydro-3aH-[1,3]dioxolo[4,5-c]pyran-2-one, or any of the following structures: 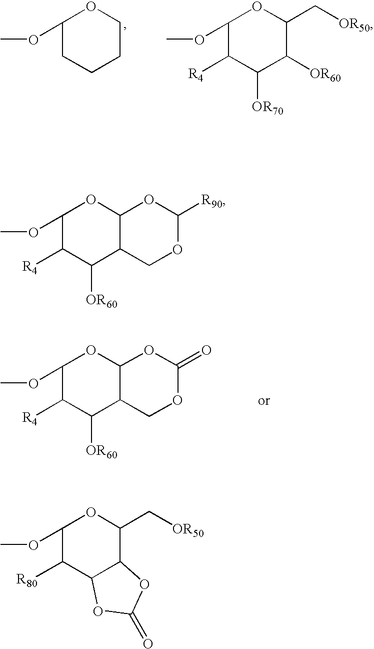
wherein R₅₀, R₆₀ and R₇₀ are independently C₁-C₆ alkyl, C₀-C₆ alkylaryl, acyl, C₀-C₆ alkyl-C(═O)aryl, C₀-C₆ alkyl-C(═O)-C₁-C₆ alkyl, acetyl, benzoyl, benzyl, tert-butyldimethylsilyl, tert-butyldiphenylsilyl, hydrogen, or —Si(R₁₀₀)₃, wherein each R₁₀₀ is the same or different and is C₁-C₆ alkyl or C₀-C₆ alkylaryl; R₈₀ is C₁-C₆ alkyl, methyl or hydrogen, and R₉₀ is —H, or any alkyl chain or aryl group or substituted aryl group. For example,
R₅₀, R₆₀ and R₇₀ are independently 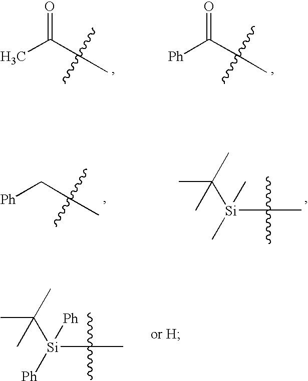
R₈₀ is —H or —CH₃; and
R₉₀ is C₁-C₆ alkyl, aryl or substituted aryl.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as t-butyl, isopropyl, propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-arbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 15 carbon atoms (C₁-C₁₅ alkyl), more preferably from 1 to 10 carbon atoms (C₁-C₁₀ alkyl) and even more preferably from 1 to 6 carbon atoms (C₁-C₆ alkyl or lower alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl , prop-1-en-2-yl, prop-2-en-1-yl (allyl), prop-2-en-2-yl, cycloprop-1-en-1-yl; cycloprop-2-en-1-yl ; butenyls such as but-1-en-1-yl, but-1-en-2-yl, 2-methyl-prop-1-en-1-yl, but-2-en-1-yl , but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-hain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” by itself or as part of another substituent refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1 ,2-diyl, prop-1-en-i,1-diyl, prop-1-en-1 ,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-i,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyidiyl and/or alkynyldiyl is used. Where it is specifically intended that the two valencies are on the same carbon atom, the nomenclature “alkylidene” is used. In preferred embodiments, the alkyldiyl group comprises from 1 to 6 carbon atoms (C1-C6 alkyldiyl). Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno,” by itself or as part of another substituent, refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[l]yno, but[2]yno, buta[1,3]diyno, etc.; and the like. Where specific levels of saturation are intended, the nomenclature alkano, alkeno and/or alkyno is used. In preferred embodiments, the alkyleno group is (C₁-C₆) or (C₁-C₃) alkyleno. Also preferred are straight-chain saturated alkano groups, e.g., methano, ethano, propano, butano, and the like.

“Alkoxy,” by itself or as part of another substituent, refers to a radical of the formula —OR, where R is an alkyl or cycloalkyl group as defined herein. Representative examples alkoxy groups include, but are not limited to, methoxy, ethoxy, propoxy, isopropoxy, butoxy, tert-butoxy, cyclopropyloxy, cyclopentyloxy, cyclohexyloxy and the like.

“Alkoxycarbonyl,” by itself or as part of another substituent, refers to a radical of the formula —C(O)-alkoxy, where alkoxy is as defined herein.

“Alkylthio,” by itself or as part of another substituent, refers to a radical of the formula —SR, where R is an alkyl or cycloalkyl group as defined herein. Representative examples of Alkylthio groups include, but are not limited to, methylthio, ethylthio, propylthio, isopropylthio, butylthio tert-butylthio, cyclopropylthio, cyclopentylthio, cyclohexylthio, and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms (C₆-C₂₀ aryl), more preferably from 6 to 15 carbon atoms (C₆-C₁₅ aryl) and even more preferably from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl, more preferably, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl, and even more preferably, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryloxy,” by itself or as part of another substituent, refers to a radical of the formula —O-aryl, where aryl is as defined herein.

“Arylalkyloxy, by itself or as part of another substituent, refers to a radical of the formula —O-arylalkyl, where arylalkyl is as defined herein.

“Aryloxycarbonyl,” by itself or as part of another substituent, refers to a radical of the formula —C(O)—O-aryl, where aryl is as defined herein.

“Carbamoyl,” by itself or as part of another substituent, refers to a radical of the formula —C(O)NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. Preferably, the cycloalkyl group comprises from 3 to 10 ring atoms (C₃-C₁₀ cycloalkyl) and more preferably from 3 to 7 ring atoms (C₃-C₇ cycloalkyl). The cycloalkyl group also includes polycyclic groups such as, but not limited to, adamantane, and the like.

“Cycloheteroalkyl, ” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. Preferably, the cycloheteroalkyl group comprises from 3 to 10 ring atoms (3-10 membered cycloheteroalkyl) and more preferably from 5 to 7 ring atoms (5-7 membered cycloheteroalkyl).

A cycloheteroalkyl group may be substituted at a heteroatom, for example, a nitrogen atom, with a lower alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteralkyl group may be attached to the remainder of the molecule via a ring carbon atom or a ring heteroatom.

The term “heterocycle” as used herein mean a cycloheteroalkyl, heteroaryl or parent heteroaromatic ring system. Heterocycle includes groups that are, for example, saturated, unsaturated, or aromatic heteroatomic ring systems.

“Dialkylamino” or “Monoalkylamino,” by themselves or as part of other substituents, refer to radicals of the formula —NRR and —NHR, respectively, where each R is independently selected from the group consisting of alkyl and cycloalkyl, as defined herein. Representative examples of dialkylamino groups include, but are not limited to, dimethylamino, methylethylamino, di 1-methylethyl)amino, (cyclohexyl)(methyl)amino, (cyclohexyl)(ethyl)amino, (cyclohexyl)(propyl)amino and the like. Representative examples of monalkylamino groups include, but are not limited to, methylamino, ethylamino, propylamino, isopropylamino, cyclohexylamino, and the like.

“Halogen” or “Halo,” by themselves or as part of another substituent, refer to a fluoro, chloro, bromo and/or iodo radical.

“Haloalkyl,” by itself or as part of another substituent, refers to an alkyl group as defined herein in which one or more of the hydrogen atoms is replaced with a halo group. The term “haloalkyl” is specifically meant to include monohaloalkyls, dihaloalkyls, trihaloalkyls, etc. up to perhaloalkyls. The halo groups substituting a haloalkyl can be the same, or they can be different. For example, the expression “(C₁-C₂) haloalkyl” includes 1-fluoromethyl, 1-fluoro-2-chloroethyl, difluoromethyl, trifluoromethyl, 1-fluoroethyl, 1,1-difluoroethyl, 1,2-difluoroethyl, 1,1,1-trifluoroethyl, perfluoroethyl, etc.

“Haloalkyloxy,” by itself or as part of another substituent, refers to a group of the formula —O-haloalkyl, where haloalkyl is as defined herein.

“Heteroalkyl,” “Heteroalkanyl,” “Heteroalkenyl,” “Heteroalkynyl,” “Heteroalkyldiyl” and “Heteroalkyleno,” by themselves or as part of other substituents, refer to alkyl, alkanyl, alkenyl, alkynyl, alkyidiyl and alkyleno groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O), —S(O)₂-, —S(O)NH—, —S(O)₂NH— and the like and combinations thereof. The heteroatoms or heteroatomic groups may be placed at any interior position of the alkyl, alkenyl or alkynyl groups. Examples of such heteroalkyl, heteroalkanyl, heteroalkenyl and/or heteroalkynyl groups include —CH₂—CH₂—O—CH₃, —CH₂—CH₂—NH—CH₃, —CH₂—CH₂—N(CH₃)—CH₃, —CH₂—S—CH₂, —CH₃, —CH₂—CH₂—S(O)—CH₃, —CH₂—CH₂—S(O)₂—CH₃, —CH═CH—O—CH₃, —CH₂—CH═N—O—CH₃, and —CH₂—CH₂—O—C═CH. For heteroalkyldiyl and heteroalkyleno groups, the heteratom or heteratomic group can also occupy either or both chain termini. For such groups, no orientation of the group is implied.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, [symbol] -carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group comprises from 5 to 20 ring atoms (5-20 membered heteroaryl), more preferably from 5 to 10 ring atoms (5-10 membered heteroaryl). Preferred heteroaryl groups are those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In preferred embodiments, the heteroarylalkyl group is a 6-21 membered heteroarylalkyl, e.g., the alkanyl, alkenyl or alkynyl moiety of the heteroarylalkyl is (C1-C6) alkyl and the heteroaryl moiety is a 5-15-membered heteroaryl. In particularly preferred embodiments, the heteroarylalkyl is a 6-13 membered heteroarylalkyl, e.g., the alkanyl, alkenyl or alkynyl moiety is (C1-C3) alkyl and the heteroaryl moiety is a 5-10 membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, f,carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutically acceptable salt” refers to a salt of a compound of the invention which is made with counterions understood in the art to be generally acceptable for pharmaceutical uses and which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 344-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge etal., 1977, J. Pharm. Sci. 66:1-19).

“Pharmaceutically acceptable vehicle” refers to a diluent, adjuvant, excipient or carrier that is acceptable for human use.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 18, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (e.g., methyl and ethyl esters, acetate or propionate groups or glycol esters) or alkylated such as benzyl and trityl ethers, as well as alkyl ethers, tetrahydropyranyl ethers, trialkylsilyl ethers (e.g., TMS or TIPPS groups) and allyl ethers.

“Progroup” refers to a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃. Various phosphonate, ester and dioxolenone progroups and their uses are described herein.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O—, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —CH₂—P(O)(OR^{b})(OR^{b}), —O—P(O)(OR^{b})(OR^{b}), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 5-, 6- or 7-membered cycloheteroalkyl which may optionally include from 1 to 4 of the same or different additional heteroatoms selected from the group consisting of O, N and S. As specific examples, -NRCRc is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a} optionally substituted with C₁-C₄-alkyl or C(O)OR^{b}, halo, —O⁻, —O—(CH₂)₀₋₄—R^{b}, —SR^{b}, S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)2O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —O—C(O)—NH—R^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined. Additional substituent groups for substituting unsaturated carbon atoms are C₁-C₄-alkyl moieties substituted with one of the foregoing moieties.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Sulfamoyl,” by itself or as part of another substituent, refers to a radical of the formula —S(O)₂NR′R″, where R′ and R″ are each, independently of one another, selected from the group consisting of hydrogen, alkyl and cycloalkyl as defined herein, or alternatively, R′ and R″, taken together with the nitrogen atom to which they are bonded, form a 5-, 6- or 7-membered cycloheteroalkyl ring as defined herein, which may optionally include from 1 to 4 of the same or different additional heteroatoms selected from the group consisting of O, S and N.

Some examples of prodrugs of the invention are provided in TABLES 1 through 13. These examples merely serve to illustrate some embodiments of the invention and in no way limit the scope of the invention. Also included in the invention are the various regioisomers and hydro isomers of the prodrugs described herein, including the various regioisomers and hydro isomers of the prodrugs of structural formulae (I) through (XI) and TABLES 1 through 13.

TABLE 1

| Cpd No. | Structure |
|---|---|
| 1008 | 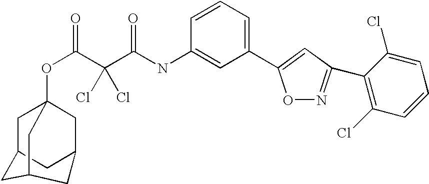 |
| 1009 | 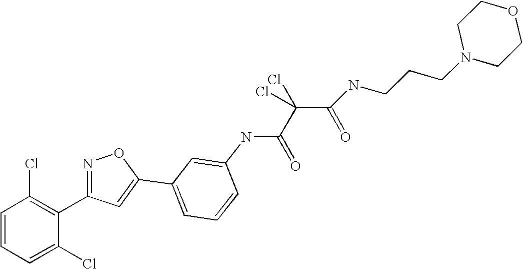 |
| 1010 | 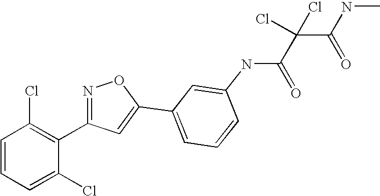 |
| 1011 | 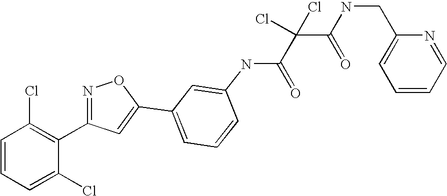 |
| 1012 | 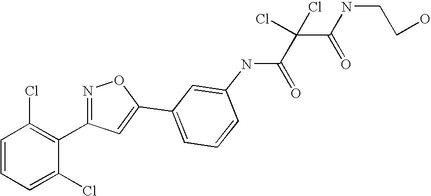 |
| 1013 | 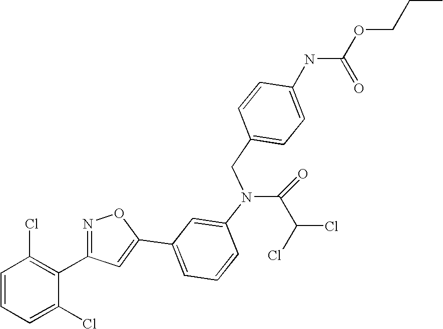 |
| 1014 | 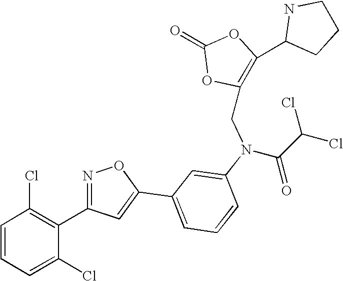 |
| 1015 | 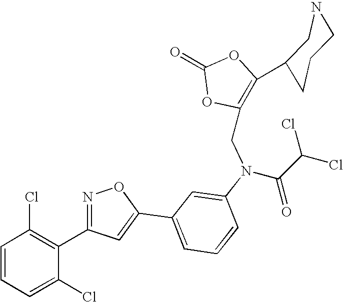 |
| 1016 | 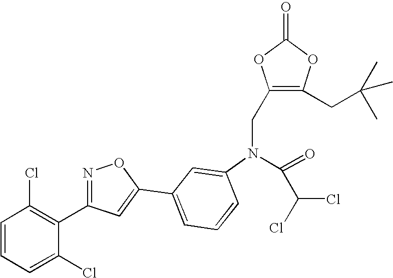 |
| 1017 | 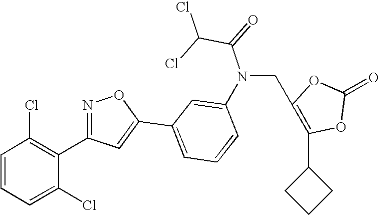 |
| 1018 | 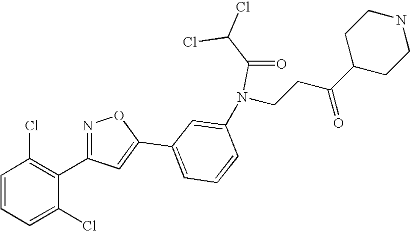 |
| 1019 | 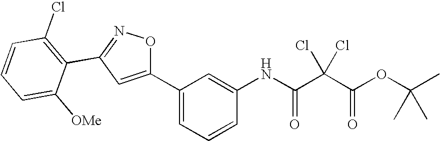 |
| 1020 | 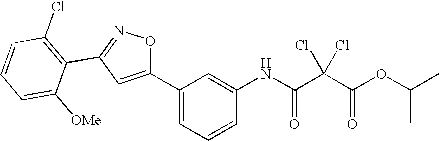 |
| 1021 | 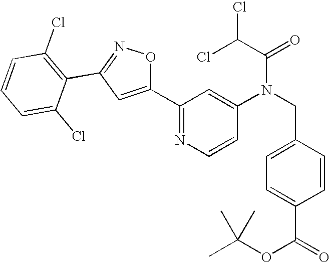 |
| 1022 | 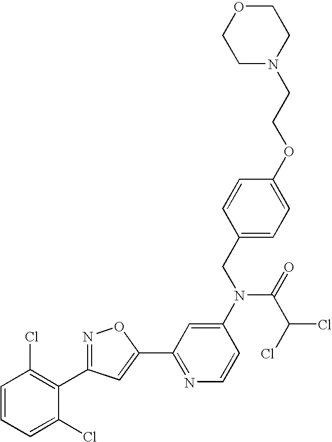 |
| 1023 | 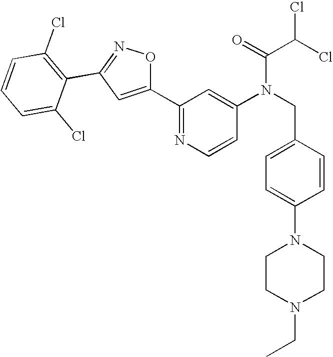 |
| 1024 | 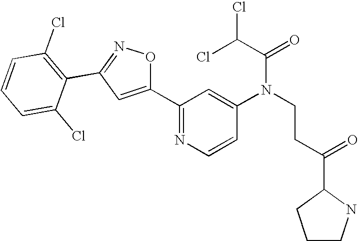 |
| 1025 | 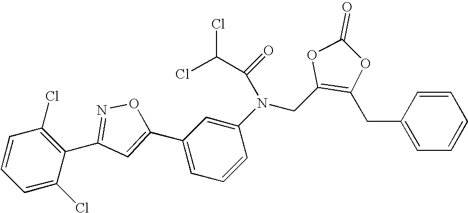 |
| 1026 | 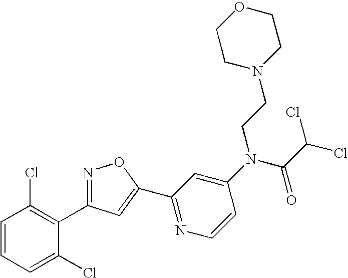 |
| 1027 | 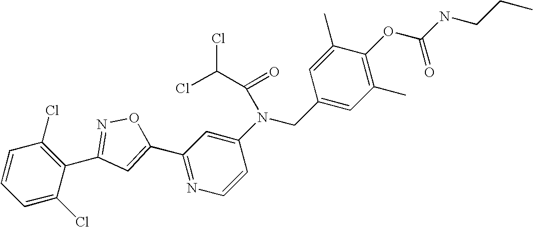 |
| 1028 | 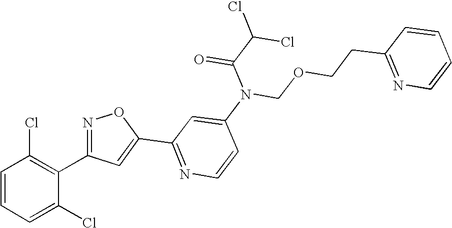 |
| 1029 | 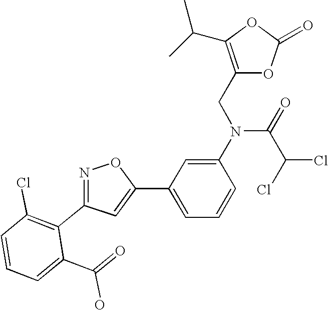 |
| 1030 | 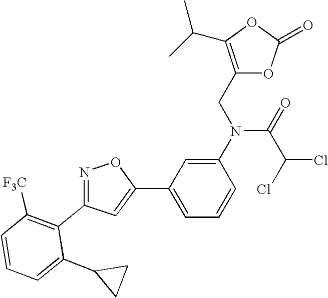 |
| 1031 | 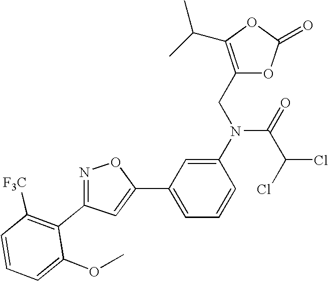 |
| 1032 | 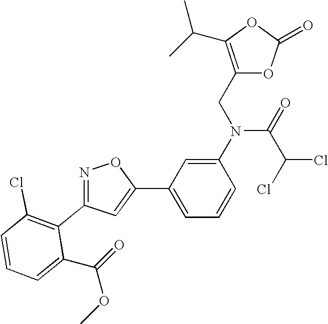 |
| 1033 | 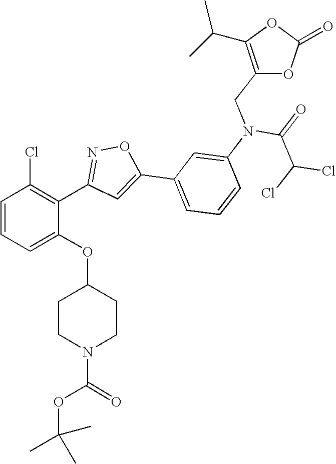 |
| 1034 | 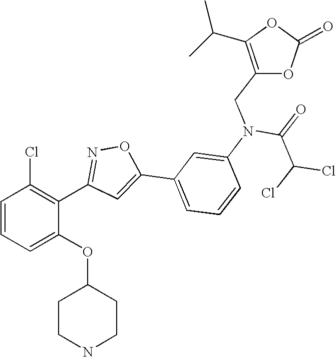 |
| 1035 | 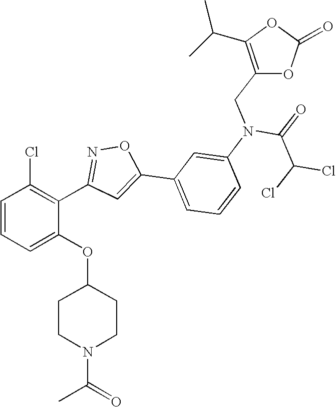 |
| 1036 | 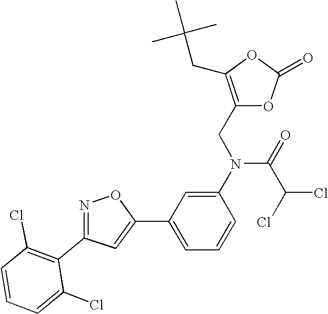 |
| 1037 | 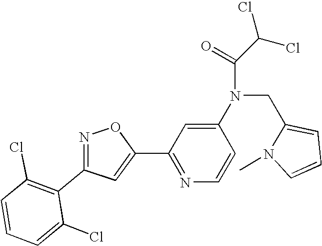 |
| 1038 | 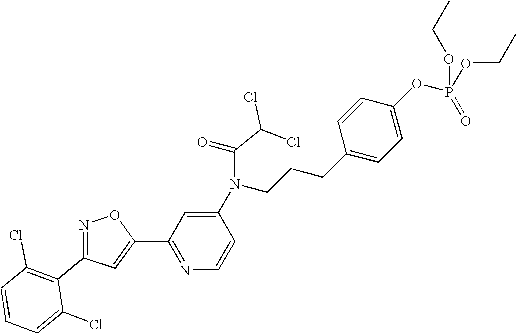 |
| 1039 | 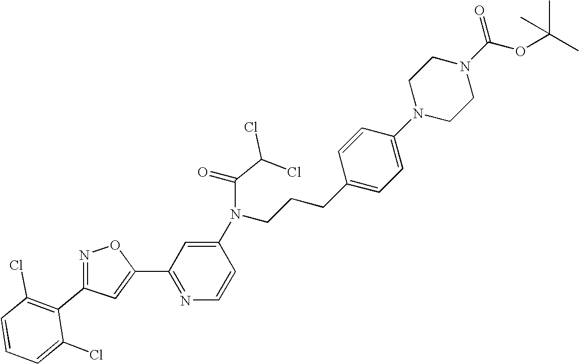 |
| 1040 | 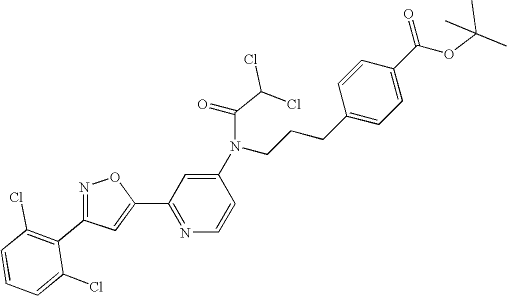 |
| 1041 | 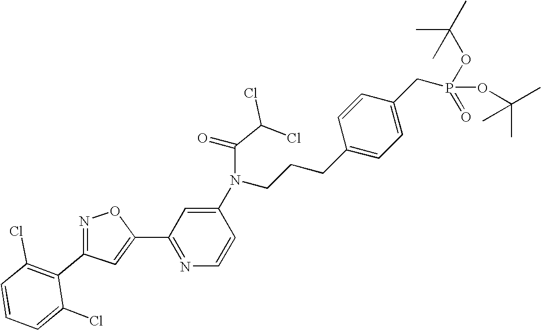 |
| 1042 | 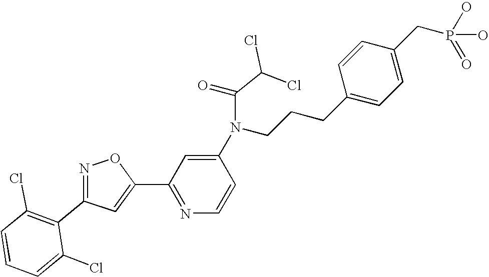 |
| 1043 | 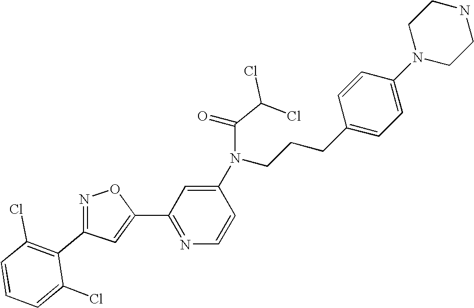 |
| 1044 | 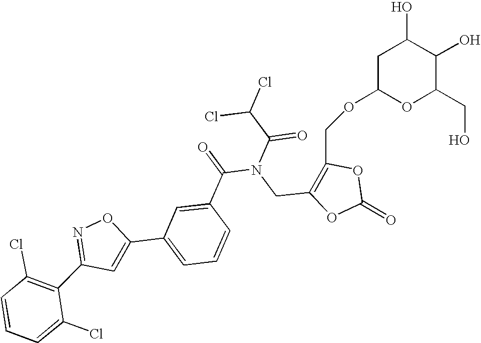 |
| 1045 | 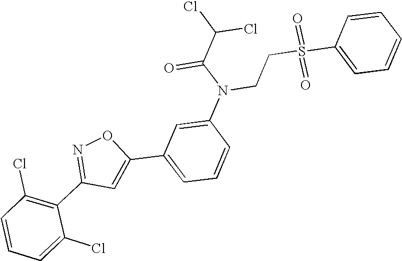 |
| 1049 | 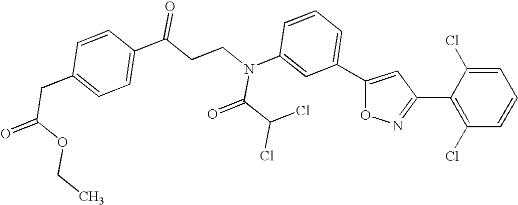 |
| 1046 | 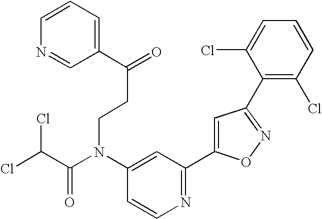 |
| 1050 | 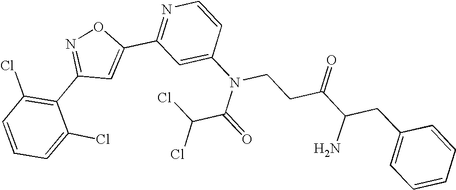 |
| 1051 | 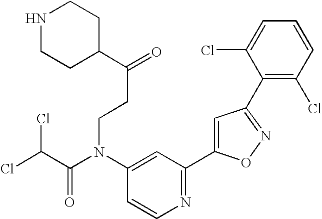 |
| 1052 | 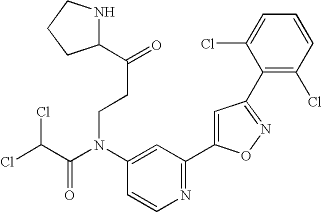 |
| 1053 | 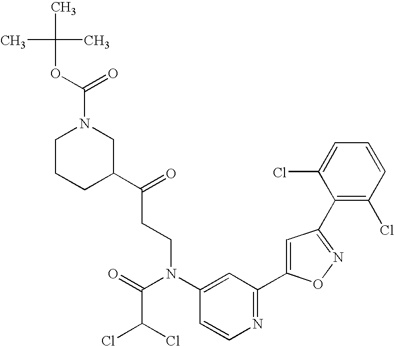 |
| 1054 | 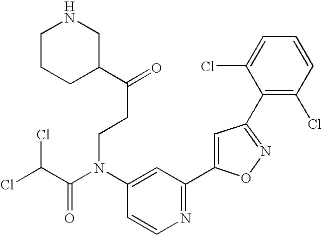 |
| 1055 | 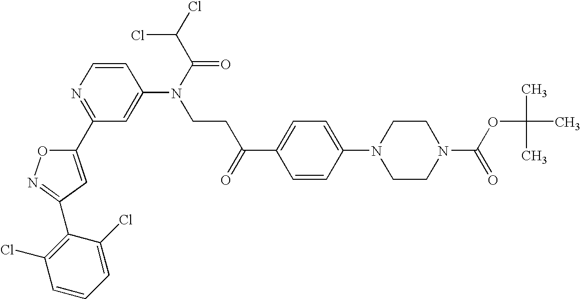 |
| 1056 | 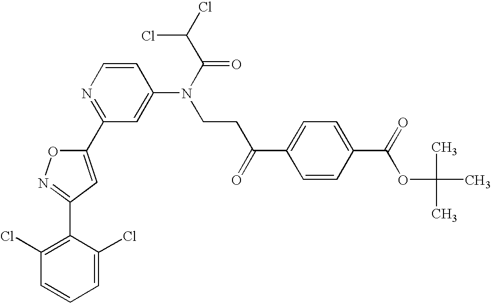 |
| 1047 | 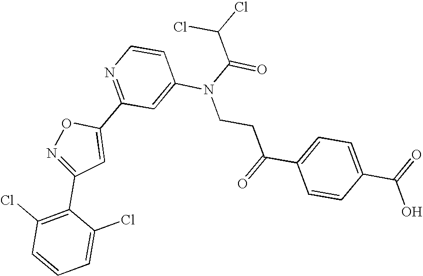 |
| 1048 | 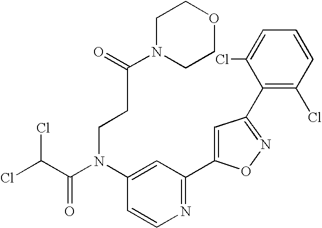 |
| 1057 | 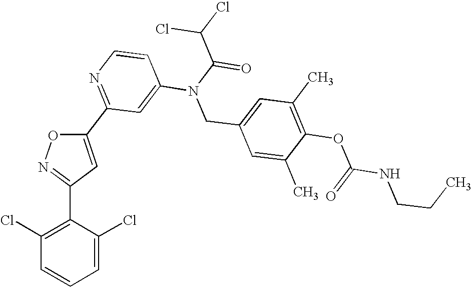 |

TABLE 2

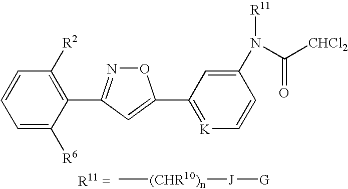

| # | R² | R⁶ | K | R¹⁰ | n | J | G |
|---|---|---|---|---|---|---|---|
| 311a | Cl | Cl | —CH— | H | 2 | —C(O)— | 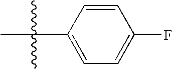 |
| 101a | Cl | Cl | —CH— | H | 2 | —SO₂— | 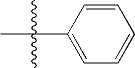 |
| 102a | Cl | Cl | —CH— | H | 2 | —C(O)— | 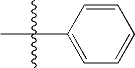 |
| 103a | Cl | Cl | —CH— | H | 2 | —C(O)— | 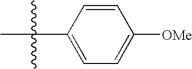 |
| 104a | Cl | Cl | —CH— | H | 2 | —C(O)— | 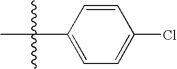 |
| 105a | Cl | Cl | —CH— | H | 1 | —C(O)— | 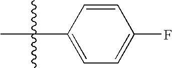 |
| 106a | Cl | Cl | —CH— | H | 2 | —CH₂— | 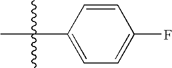 |
| 407a | Cl | Cl | —CH— | CH₃, H * | 2 | —C(O)— | 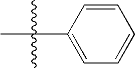 |
| 108a | Cl | Cl | —CH— | H | 2 | —C(NOCH₃)— | 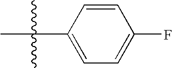 |
| 109a | Cl | Cl | —CH— | H | 2 | —CH(OH)— | 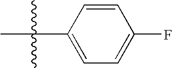 |
| 110a | Cl | Cl | —CH— | H | 2 | —C(O)— | 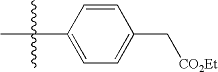 |
| 111a | Cl | Cl | —CH— | H | 2 | —C(O)— | 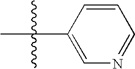 |
| 112a | Cl | Cl | —N— | H | 2 | —C(O)— | 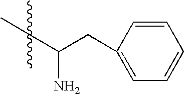 |
| 113a | OMe | Cl | —CH— | H | 2 | —C(O)— | 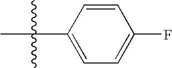 |
| 114a | CF₃ | 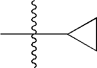 | —CH— | H | 2 | —C(O)— | 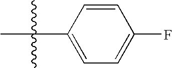 |
| 115a | Cl | Cl | —N— | | 0 | —CH₂— | 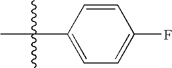 |
| 116a | Cl | Cl | —N— | H | 1 | —CH₂— | 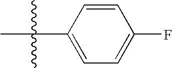 |
| 117a | Cl | Cl | —CH— | | 0 | —CH₂— | 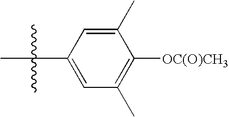 |
| 118a | Cl | Cl | —N— | | 0 | —CH₂— | 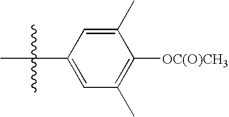 |
| 119a | Cl | Cl | —CH— | | 0 | —CH₂— | 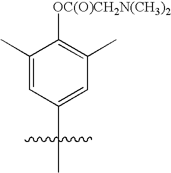 |
| 120a | Cl | Cl | —N— | | 0 | —CH₂— | 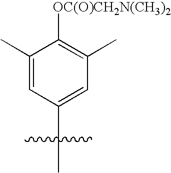 |
| 121a | Cl | Cl | —CH— | | 0 | —CH₂— | 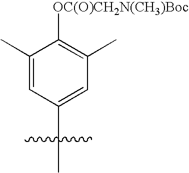 |
| 122a | Cl | Cl | —N— | | 0 | —CH₂— | 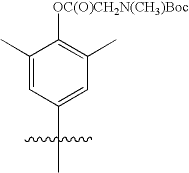 |
| 123a | Cl | Cl | —CH— | | 0 | —CH₂— | 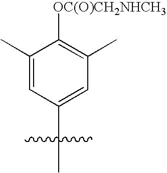 |
| 124a | Cl | Cl | —N— | | 0 | —CH₂— | 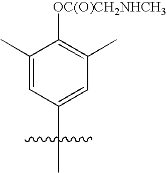 |
| 125a | Cl | Cl | —CH— | | 0 | —CH₂— | 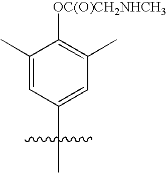 |
| 605a | Cl | Cl | —N— | H | 2 | —C(O)— | 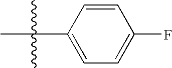 |
| 127a | Cl | 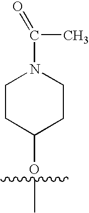 | —CH— | H | 2 | —C(O)— | 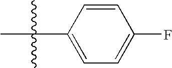 |
| 128a | Cl | 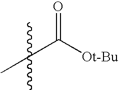 | —CH— | H | 2 | —C(O)— | 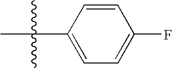 |
| 129a | Cl | 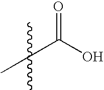 | —CH— | H | 2 | —C(O)— | 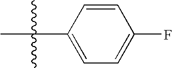 |
| 130a | Cl | Cl | —CH— | H | 2 | —C(O)— | 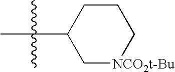 |
| 131a | Cl | Cl | —CH— | H | 2 | —C(O)— | 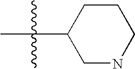 |
| 132a | Cl | Cl | —N— | H | 2 | —CH₂— | 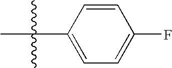 |
| 133a | Cl | 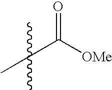 | —CH— | H | 2 | —C(O)— | 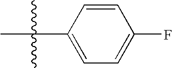 |
| 134a | Cl | Cl | —N— | H | 2 | —C(O)— | 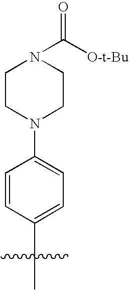 |
| 135a | Cl | Cl | —N— | H | 2 | —C(O)— | 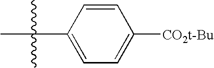 |
| 136a | Cl | Cl | —N— | H | 2 | —C(O)— | 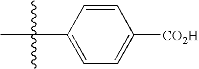 |
| 137a | Cl | Cl | —N— | H | 2 | —C(O)— | 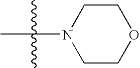 |

The * indicates that R¹⁰ is methyl when attached to the carbon atom adjacent to the nitrogen atom and is hydrogen otherwise.

The skilled artisan will further appreciate that the progroups described herein (i.e., —R¹¹ and —C(O)CHX₂) may be combined with any therapeutic agent which has a primary amine or primary sulfonamide to provide prodrugs of the therapeutic agent. Accordingly, compounds according to structural Formula (IV) are also provided herein: 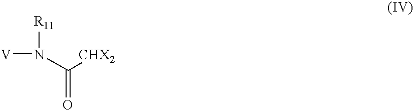
wherein R¹¹ and X are as defined, supra, and V—N is a primary amine containing therapeutic agent or a primary sulfonamide containing therapeutic agent where one of the hydrogen atoms of the amine has been replaced with —R¹¹ and the other hydrogen atom has been replaced by —C(O)CHX₂. Many such therapeutic agents (i.e., V—NH₂, V—SO₂NH₂) are known in the art and include, but are not limited to, abacavir, acadesine, acediasulfone, amiloride, aminorex, cisapride, metoclopramide, mexiletine, pamidronate, pramipexole, prazosin, procainamide, dimethoxyphenethylamine, aletamine, amphetamine, aspartame, chlortermine, dopamine, L-Dopa, etryptamine, methyldopamine, norepinephrine, norepinephrine, enviroxime, triamterene, pipedemic acid, tyleno, epirvir, lamuvidine, zidovudone, cipro, ciprofluxavir, gantavol, gantrisin, salmeterol, and similar compounds. Other therapeutic agents (i.e., V—NH₂, V—SO₂NH₂) which may be used in the current invention will be obvious to the skilled artisan.

### Uses and Administration

Owing to their ability to inhibit HCV replication, the metabolically active agents of the prodrugs of the invention and/or compositions thereof can be used in a variety of contexts. For example, the prodrugs of the invention can be used as controls in in vitro assays to identify additional more or less potent anti HCV prodrugs. As another example, the prodrugs of the invention and/or compositions thereof can be used as preservatives or disinfectants in clinical settings to prevent medical instruments and supplies from becoming infected with HCV virus. When used in this context, the prodrugs of the invention and/or composition thereof may be applied to the instrument to be disinfected at a concentration that is a multiple, for example 1×, 2×, 3×, 4×, 5× or even higher, of the measured IC₅₀ for the metabolically active agent of the prodrug.

In a specific embodiment, the prodrugs and/or compositions can be used to “disinfect” organs for transplantation. For example, a liver or portion thereof being prepared for transplantation can be perfused with a solution comprising an inhibitory prodrug of the invention prior to implanting the organ into the recipient. This method has proven successful with lamuvidine (3TC, Epivir®, Epivir-HB®) for reducing the incidence of hepatitis B virus (HBV) infection following liver transplant surgery/therapy. Quite interestingly, it has been found that such perfusion therapy not only protects a liver recipient free of HBV infection (HBV−) from contracting HBV from a liver received from an HBV+ donor, but it also protects a liver from an HBV− donor transplanted into an HBV+ recipient from attack by HBV. The prodrugs of the invention may be used in a similar manner prior to organ or liver transplantation.

The prodrugs of the invention and/or compositions thereof find particular use in the treatment and/or prevention of HCV infections in animals and humans. When used in this context, the prodrugs may be administered per se, but are typically formulated and administered in the form of a pharmaceutical composition. The exact composition will depend upon, among other things, the method of administration and will apparent to those of skill in the art. A wide variety of suitable pharmaceutical compositions are described, for example, in Remington's Pharmaceutical Sciences, 20^{th} ed., 2001).

Formulations suitable for oral administration can consist of (a) liquid solutions, such as an effective amount of the prodrugs suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The prodrug of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the prodrug of choice with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are the preferred methods of administration. A specific example of a suitable solution formulation may comprise from about 0.5-100 mg/ml prodrug and about 1000 mg/ml propylene glycol in water. Another specific example of a suitable solution formulation may comprise from about 0.5-100 mg/ml prodrug and from about 800-1000 mg/ml polyethylene glycol 400 (PEG 400) in water.

A specific example of a suitable suspension formulation may include from about 0.5-30 mg/ml prodrug and one or more excipients selected from the group consisting of: about 200 mg/ml ethanol, about 1000 mg/ml vegetable oil (e.g., corn oil), about 600-1000 mg/ml fruit juice (e.g., grape juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH 7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml prodrug, about 100-200 mg/ml lecithin (or other phospholipid or mixture of phospholipids) and optionally about 5 mg/ml cholesterol in water. For subcutaneous administration of a prodrug, a liposome suspension formulation including 5 mg/ml prodrug in water with 100 mg/ml lecithin and 5 mg/ml prodrug in water with 100 mg/ml lecithin and 5 mg/ml cholesterol provides good results. This formulation may be used for other prodrugs of the invention.

The formulations of prodrugs can be presented in unit-dose or multi-dose sealed containers, such as ampoules and vials. Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the prodrug. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail, below.

In therapeutic use for the treatment of HCV infection, the prodrugs utilized in the pharmaceutical method of the invention are administered to patients diagnosed with HCV infection at dosage levels suitable to achieve therapeutic benefit. By therapeutic benefit is meant that the administration of prodrug leads to a beneficial effect in the patient over time. For example, therapeutic benefit is achieved when the HCV titer or load in the patient is either reduced or stops increasing. Therapeutic benefit is also achieved if the administration of prodrug slows or halts altogether the onset of the organ damage that or other adverse symptoms typically accompany HCV infections, regardless of the HCV titer or load in the patient.

The prodrugs of the invention and/or compositions thereof may also be administered prophylactically in patients who are at risk of developing HCV infection, or who have been exposed to HCV, to prevent the development of HCV infection. For example, the prodrugs of the invention and/or compositions thereof may be administered to hospital workers accidentally stuck with needles while working with HCV patients to lower the risk of, or avoid altogether, developing an HCV infection.

Initial dosages suitable for administration to humans may be determined from in vitro assays or animal models. For example, an initial dosage may be formulated to achieve a serum concentration that includes the IC₅₀ of the particular metabolically active agent of the prodrug being administered, as measured in an in vitro assay. Alternatively, an initial dosage for humans may be based upon dosages found to be effective in animal models of HCV infection. Suitable model systems are described, for example, in Muchmore, 2001, Immunol. Rev. 183:86-93 and Lanford & Bigger, 2002, Virology, 293:1-9, and the referenced cited therein. As one example, the initial dosage may be in the range of about 0.01 mg/kg/day to about 200 mg/kg/day, or about 0.1 mg/kg/day to about 100 mg/kg/day, or about 1 mg/kg/day to about 50 mg/kg/day, or about 10 mg/kg/day to about 50 mg/kg/day, can also be used. The dosages, however, may be varied depending upon the requirements of the patient, the severity of the condition being treated, and the prodrug being employed. The size of the dose also will be determined by the existence, nature, and extent of any adverse side-effects that accompany the administration of a particular prodrug in a particular patient. Determination of the proper dosage for a particular situation is within the skill of the practitioner. Generally, treatment is initiated with smaller dosages which are less than the optimum dose of the prodrug. Thereafter, the dosage is increased by small increments until the optimum effect under circumstances is reached. For convenience, the total daily dosage may be divided and administered in portions during the day, if desired.

### Combination Therapy

In certain embodiments of the present invention, the prodrugs of the invention and/or compositions thereof can be used in combination therapy with at least one other therapeutic agent. A prodrug of the invention and/or composition thereof and the therapeutic agent can act additively or, more preferably, synergistically. The prodrug of the invention and/or a composition thereof may be administered concurrently with the administration of the other therapeutic agent(s), or it may be administered prior to or subsequent to administration of the other therapeutic agent(s).

In one embodiment, the prodrugs of the invention and/or compositions thereof are used in combination therapy with other antiviral agents or other therapies known to be effective in the treatment or prevention of HCV. As a specific example, the prodrugs of the invention and/or compositions thereof may be used in combination with known antivirals, such as ribavirin (see, e.g., U.S. Pat. No. 4,530,901). As another specific example, the prodrugs of the invention and/or compositions thereof may also be administered in combination with one or more of the compounds described in any of the following: U.S. Pat. No. 6,143,715; U.S. Pat. No. 6,323,180; U.S. Pat. No. 6,329,379; U.S. Pat. No. 6,329,417; U.S. Pat. No. 6,410,531; U.S. Pat. No. 6,420,380; and U.S. Pat. No. 6,448,281.

Yet another specific example, the prodrugs of the invention and/or compositions thereof may be used in combination with interferons such as α-interferon, β-interferon and/or y-interferon. The interferons may be unmodified, or may be modified with moieties such as polyethylene glycol (pegylated interferons). Many suitable unpegylated and pegylated interferons are available commercially, and include, by way of example and not limitation, recombinant interferon alpha-2b such as Intron-A interferon available from Schering Corporation, Kenilworth, N.J., recombinant interferon alpha-2a such as Roferon interferon available from Hoffmann-La Roche, Nutley, N.J., recombinant interferon alpha-2C such as Berofor alpha 2 interferon available from Boehringer Ingelheim Pharmaceutical, Inc., Ridgefield, Conn., interferon alpha-nl, a purified blend of natural alpha interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 (especially Examples 7, 8 or 9 thereof) and the specific product available from Amgen, Inc., Newbury Park, Calif., or interferon alpha-n3 a mixture of natural alpha interferons made by Interferon Sciences and available from the Purdue Frederick Co., Norwalk, Conn., under the Alferon Tradename, pegylated interferon-2b available from Schering Corporation, Kenilworth, N.J. under the tradename PEG-Intron A and pegylated interferon-2a available from Hoffmann-LaRoche, Nutley, N.J. under the tradename Pegasys.

As yet another specific example, the prodrugs of the invention and/or compositions thereof may be administered in combination with both ribovirin and an interferon.

### Methods of Synthesis

The prodrugs of the invention may be obtained via synthetic methods illustrated in FIGS. 1-36. It should be understood that in FIGS. 1-36, R², R⁶, R¹⁶, R¹⁸ and R¹⁹ are as previously defined for structural formulae herein.

Starting materials useful for preparing prodrugs of the invention and intermediates thereof are commercially available or can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquefte, “Encyclopedia of Reagents for Organic Synthesis,” 3d Edition, John Wiley & Sons, 1995). Other methods for synthesis of the compounds described herein and/or starting materials are either described in the art or will be readily apparent to the skilled artisan. Accordingly, the synthetic methods and strategy presented herein are illustrative rather than comprehensive.

It should be understood that the following general methods are meant to be illustrative. For example, the synthetic routes described focus on isoxazole “B” rings. However, heterocyclic rings in place of an isoxazole “B” ring as described throughout the specification can be employed by the skilled artisan. Additionally, substituents utilized in the Figures correspond to those described throughout the specification. Manipulation and choice of substituents are within the knowledge of a skilled artisan.

One method for synthesizing substituted diphenyl isoxazoles according to structural formula (VI) or phenylpyridyl isoxazoles according to structural formula (VIII) (in either case, when D is N, E is O and F is —CH—) is provided in FIG. 1. Referring to FIG. 1, condensation of ethynyl(hetero)aromatics 201 with 2, 2-dihalo-24disubstitutedoxyphosphonyl)acetyl chloride 203 under basic conditions, provides N-substituted-ethynyl(hetero)aromatics 205. Treatment of 205 with 2,6-disubstituted-N-hydroxybenzenecarboximidoyl chloride 207 under basic conditions provides phosphonate ester containing 3,5-disubstituted isoxazoles 209, which may be optionally de-esterified with trimethylsilyl bromide to yield the phosphonic acid containing 3,5-disubstituted isoxazoles 211.

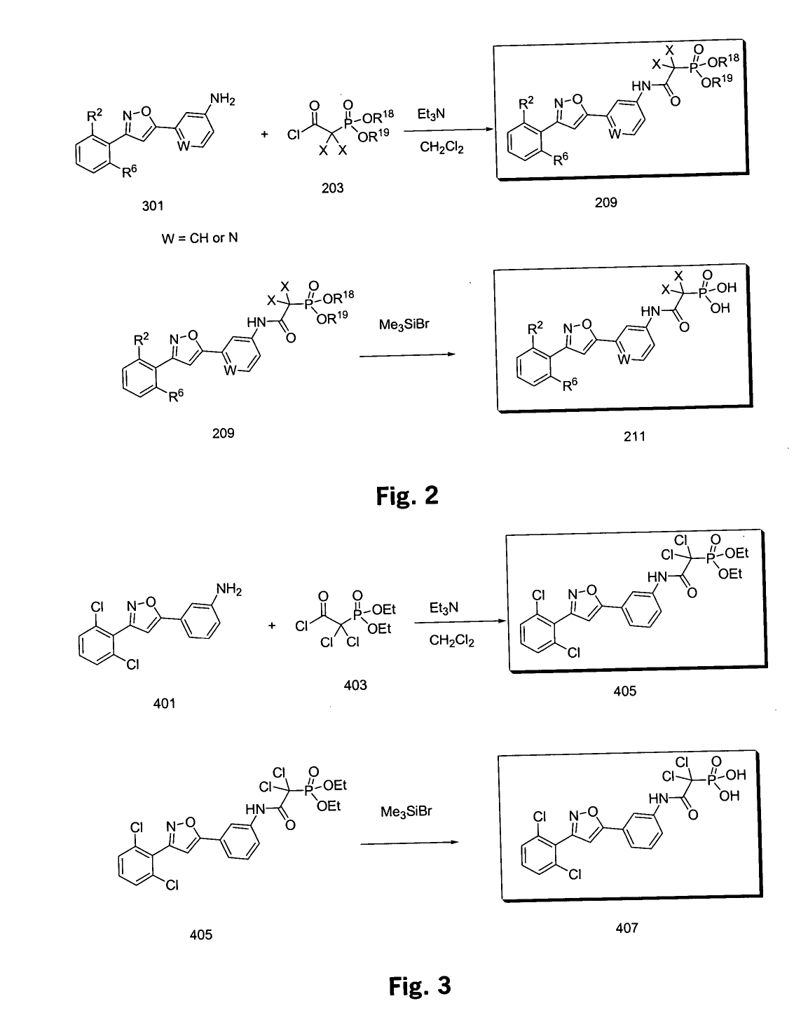

A different method for synthesizing substituted diphenyl isoxazoles according to structural formula (VI) or phenyl-pyridyl isoxazoles according to structural formula (VIII) (in either case, when D is N, E is O and F is —CH—) is provided in FIG. 2. Referring to FIG. 2, condensation of 3,5-disubstituted isoxazoles 301 with 2, 2-dihalo-24disubstitutedoxyphosphonyl)acetyl chloride 203 under basic conditions, provides phosphonate ester containing 3,5-disubstituted isoxazoles 209. Optional treatment of 209 with trimethylsilyl bromide provides the phosphonic acid containing 3,5-disubstituted isoxazoles 211. Preparation of 301 and similar compounds is known in the art can be prepared by the methods disclosed in U.S. Ser. No. 10/646,348, filed Aug. 22, 2003, U.S. Ser. Nos. 10/286,017, filed Nov. 1, 2002 and 10/440,349, filed May 15, 2003, WO 04/018463 and WO 03/040112, the contents of which are incorporated herein in their entirety.

A specific example of the synthetic method of FIG. 2 is illustrated in FIG. 3 for the preparation of diphenyl isoxazoles 405 and 407. Preparation of 401 and similar compounds is known in the art and can be prepared by the methods disclosed in U.S. Ser. Nos. 10/286,017, filed Nov. 1, 2002 and 10/440,349, filed May 15, 2003, and WO 03/040112, the contents of which are incorporated herein in their entirety.

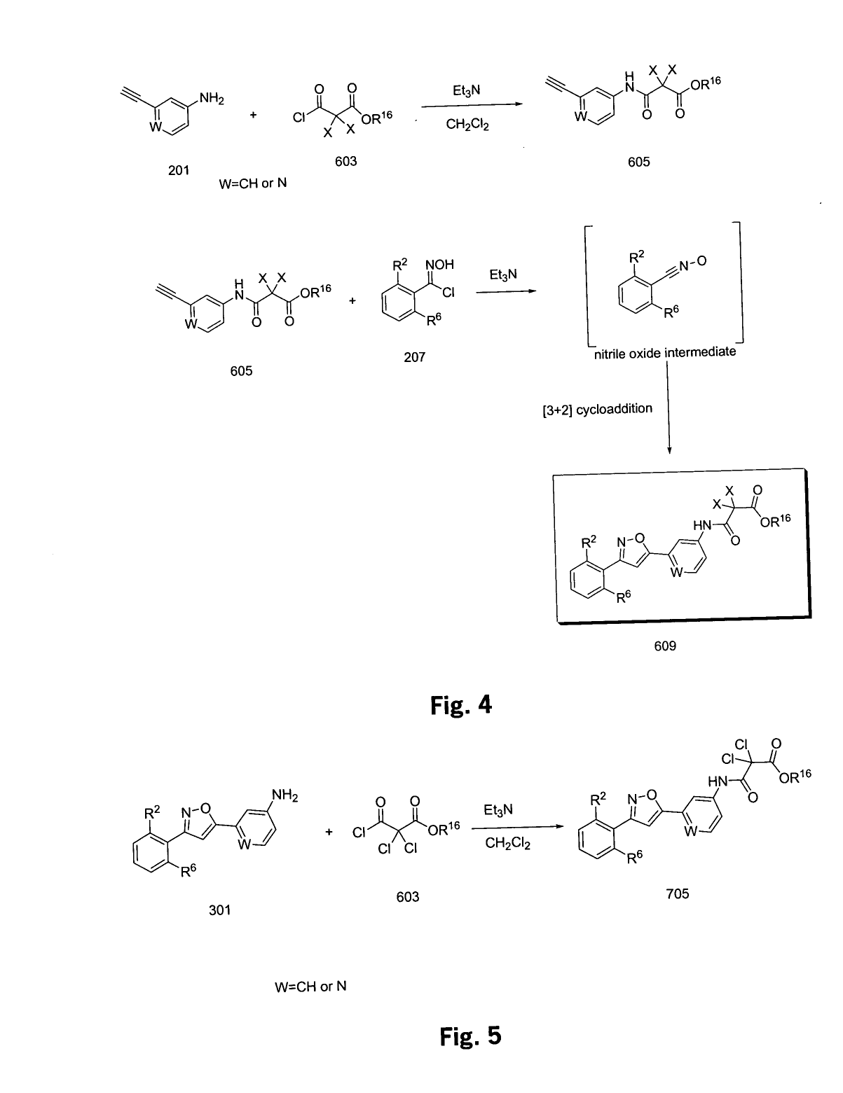

A method for synthesizing substituted diphenyl isoxazoles according to structural formula (VI) or phenyl-yridyl isoxazoles according to structural formula (VIII) (in either case, when D is N, E is O and F is —CH—) is provided in FIG. 4. Referring to FIG. 4, condensation of ethynyl(hetero)aromatics 201 with 24alkyloxycarbonylI2,2-dichloroacetyl chloride 603 under basic conditions, provides N-substituted-ethynyl(hetero)aromatics 605. Treatment of 605 with 2,6-disubstituted-N-hydroxybenzenecarboximidoyl chloride 207 under basic conditions provides carboxylate ester containing 3,5-disubstituted isoxazoles 609.

Another method for synthesizing substituted diphenyl isoxazoles according to structural formula (VI) or phenyl-pyridyl isoxazoles according to structural formula (VIII) (in either case, when D is N, E is O and F is —CH—) is provided in FIG. 5. Referring to FIG. 5, condensation of diphenyl/pyridyl isoxazole 301 with 24alkyloxycarbonyl)-2,2-dichloroacetyl chloride 603 under basic conditions, provides carboxylate ester containing 3,5-disubstituted isoxazoles 705.

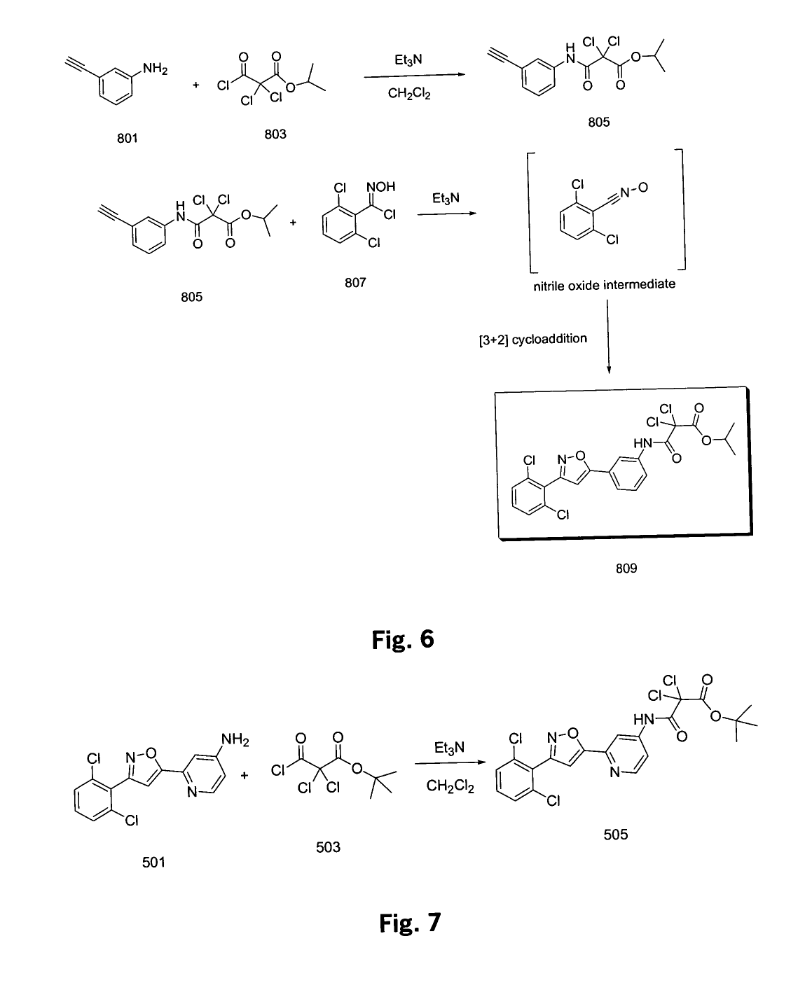

A specific example of the synthetic method of FIG. 4 is illustrated in FIG. 6 for the preparation of diphenyl isoxazole 809.

A specific example of the synthetic method of FIG. 5 is illustrated in FIG. 7 for the preparation of phenyl-pyridyl isoxazole 505. Referring to FIG. 7, condensation of pyridyl isoxazole 501 with 2-(t-butyloxycarbonyl)-2,2-dichloroacetyl chloride 503 under basic conditions, provides carboxylate ester containing 3,5-disubstituted isoxazoles 505.

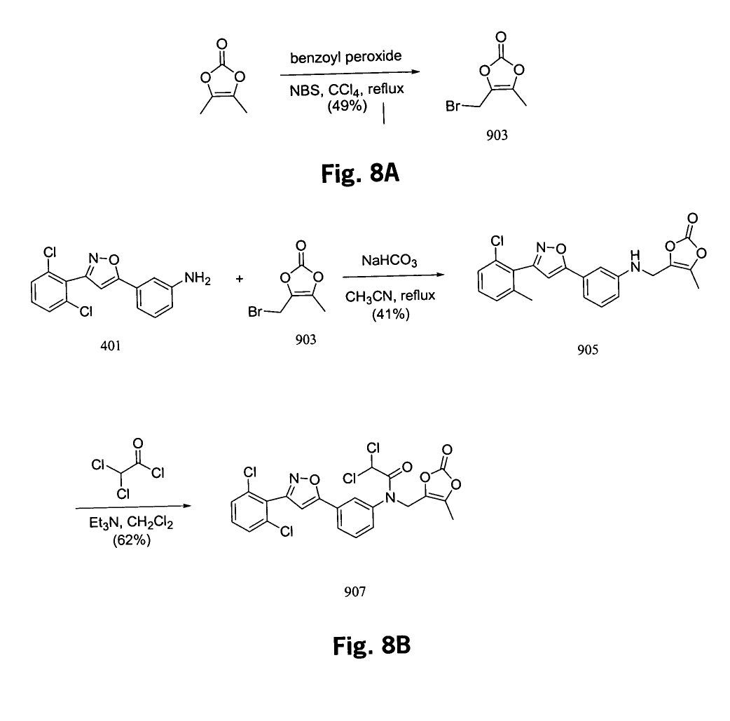

Still another method for synthesizing substituted diphenyl isoxazole according to structural formula (VI) (when D is N, E is O and F is —CH—) is provided in FIG. 8B. Referring to FIG. 8B, condensation of 3,5-disubstituted isoxazole 401 with 4-bromomethyl-5-methyl-1,3-dioxolene-2-one 903 (see FIG. 8A) under basic conditions, provides N-[3′-[3′-(2′,6′-dichlorophenyl)-5′-isoxazoyl]]N-[(5-methyl-1,3-dioxolene-2-one4-yl)methylene]aniline 905. Treatment of 905 with dichloroacetyl chloride under basic conditions provided 2,2-dichloroN-[3′-[3′-(2′,6′-dichlorophenyl)-5′-isoxazoyl]phenyl]N-[(5-methyl-1,3-dioxolene-2-one-yl)methylene] acetamide 907.

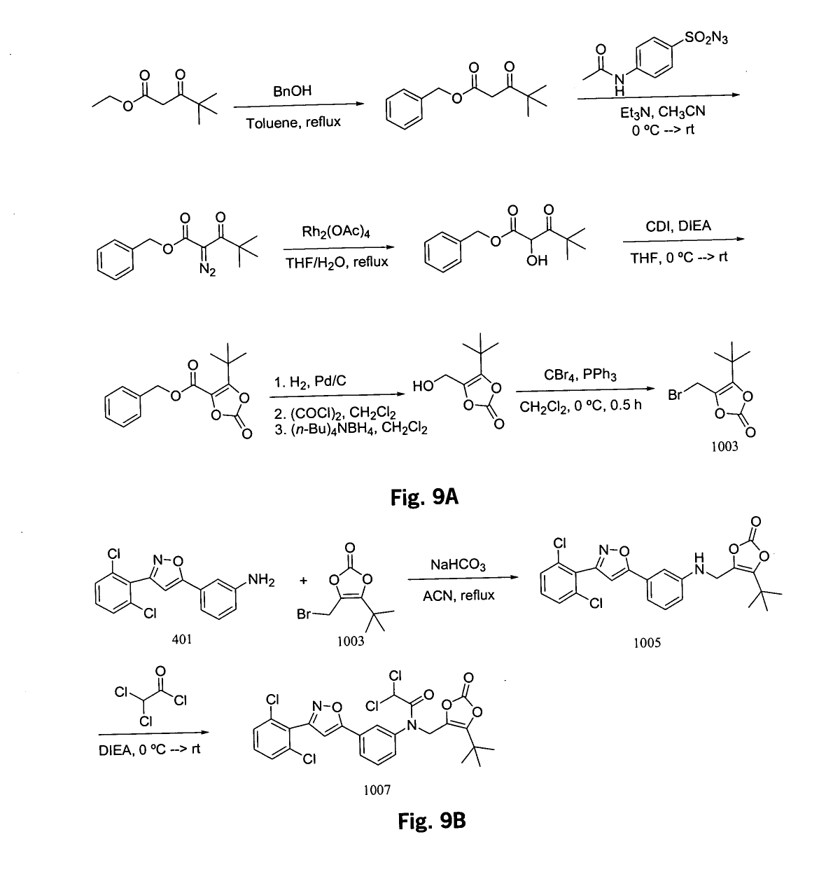

FIG. 9A depicts a method to prepare 4bromomethyl-5-t-butyl-1,3-dioxolene-2-one 1003, useful in the synthesis of 2,2-dichloroN-[3′-[3′-(2′,6′-dichlorophenyl)-5′-isoxazoyl]phenyl]-N-[(5-tbutyl-1,3-dioxolene-2-one4-yl)methylene]acetamide 1007. Referring to FIG. 9B, condensation of 3-(2,6-dichlorophenyl)-5-(3-aminophenyl)isoxazole 401 with 4-bromomethyl-5-t-butyl-1,3-dioxolene-2-one 1003 under basic conditions, provides N-[3′-[3′-(2′,6′-dichlorophenyl)-5′-isoxazolyl]phenyl]-N-[5-t-butyl-1,3-dioxolene-2-one-4-yl)methylene]aniline 1005. Treatment of 1005 with dichloroacetyl chloride under basic conditions provided 2,2-dichloro-N-[3′-[3′-(2′,6′-dichlorophenyl)-5′-isoxazoyl]phenyl]N-[(5-tbutyl-1,3-dioxolene-2-one4-yl)methylene]acetamide 1007.

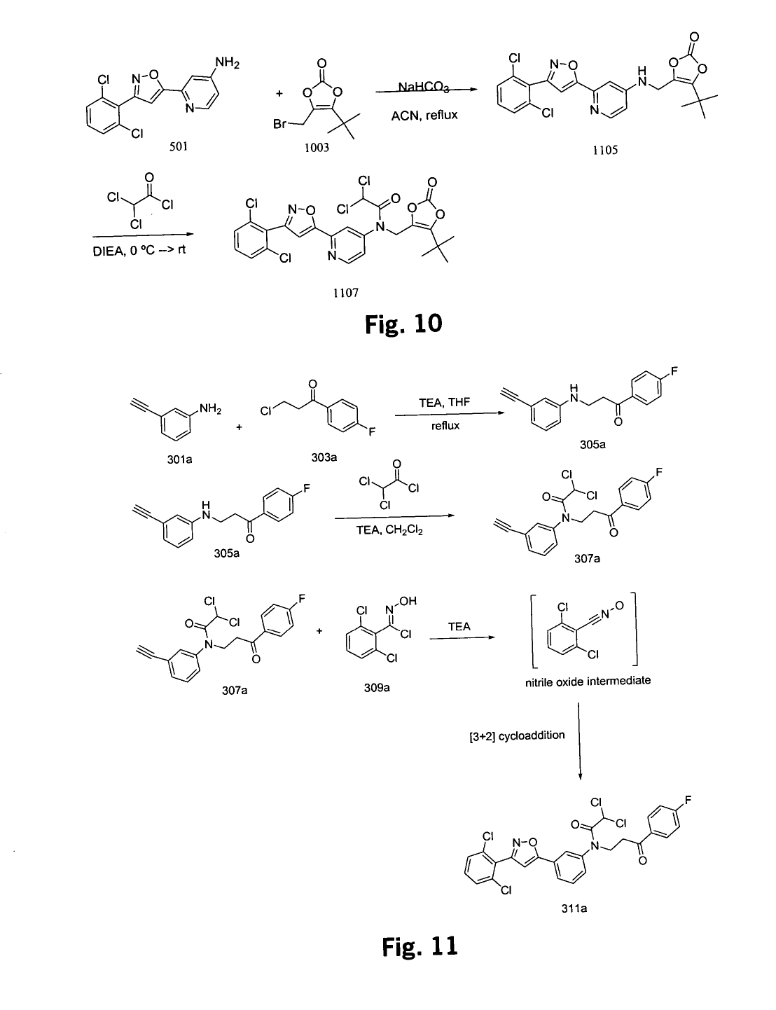

A method for synthesizing substituted phenyl-pyridyl isoxazole according to structural formula (VIII) (when D is N, E is O and F is —CH—) is provided in FIG. 10. Referring to FIG. 10, condensation of pyridyl isoxazole 501 with 4bromomethyl-5-t-butyl-1,3-dioxolene-2-one 1003 under basic conditions, provides N-[2′-[3′-(2′,6′-dichlorophenyl)-5′-isoxazolyl]4pyridyl]N-[5-t-butyl-1,3-dioxolene-2-one-4-yl)methylene]amine 1105. Treatment of 1105 with dichloroacetyl chloride under basic conditions provided 2,2-dichloroN-[3′-[3′-(2′,6′-dichlorophenyl)-5′-isoxazoyl]-2-pyridyl]-N-[(5-t-butyl-1,3-dioxolene-2-one4-yl)methylene]acetamide 1107.

The methods described in FIGS. 1, 4 and 6 above may be readily adapted for the synthesis of pyrazoles by substituting hydrazine for hydroxylamine in the reaction sequence. Further, those of skill in the art will appreciate that isoxazole regioisomers of those depicted in the above FIGS. 1-10 may be synthesized by merely interchanging the reactive functionalities of the two different aromatic rings.

In FIGS. 1-10, substituents R² and R⁶ (R²⁰'s) may include reactive functional groups that require protection during synthesis. Selection of suitable protecting groups will depend on the identity of the functional group and the synthesis method employed, and will be apparent to those of skill in the art. Guidance for selecting suitable protecting groups can be found in Greene & Wuts, supra, and the various other references cited therein.

Further guidance for carrying out 1,3-dipolar cycloaddition reactions, also named 1,3-dipolar additions, [3+2] cyclizations or [3+2] cycloadditions, can be found in “Cycloaddition Reactions in Organic Synthesis”, (Kobayashi, S. and Jorgensen, K. A., Editors), 2002, Wiley-VCH Publishers, pp. 1-332 pages (specifically, Chapters 6 and 7 on [3+2] cycloadditions and 1,3-dipolar additions, pp. 211-248 and 249-300); “1,3-Dipolar Cycloaddition”, Chemistry of Heterocyclic Compounds, Vol. 59, (Padwa, A. and Pearson, W., Editors), 2002, John Wiley, New York, pp. 1-940; “Nitrile Oxides, Nitrones, Nitronates in Organic Synthesis: Novel Strategies in Synthesis”, Torssel, K. B. G., 1988, VCH Publishers, New York, pp. 1-332; Barnes & Spriggs, 1945, J. Am. Chem Soc. 67:134; and Anjaneyulu et al., 1995, Indian J. Chem., Sect. 5 34(11):933-938).

Further guidance for synthesizing isoxazoles may be found in M. Sutharchanadevi, R. Murugan in Comprehensive Heterocyclic Chemistry II, A. R. Katritzky, C. W. Rees, E. F. V. Scriven, Eds.; Pergamon Press, Oxford, Vol. 3, p. 221; R. Grunager, P, Vita-Finzi in Heterocyclic Compounds, Vol. 49, Isoxazoles, Part one, John Wiley and Sons, New York, 1991; K. B. G. Torssell, Nitrile Oxides, Nitrones, and Nitronates in Organic Synthesis, VCH Publishers, New York, 1988; Y -Y. Ku, T. Grieme, P. Sharma, Y. -M. Pu, P. Raje, H. Morton, S. King Organic Letters, 2001, 3, 4185; V. G. Desai, S. G. Tilve Synth. Comm., 1999, 29, 3017; X. Wei, J. Fang, Y. Hu, H. Hu Synthesis, 1992, 1205; C. Kashima, N. Yoshihara, S. Shirai Heterocycles, 1981, 16, 145; A. S. R. Anjaneyulu, G. S. Rani, K. G. Annapurna, U. V. Mallavadhani, Y. L. N. Murthy Indian J. Chem. Sect B, 1995, 34, 933; R. P. Barnes, A. S. Spriggs, J. Am. Chem. Soc., 1945, 67, 134; A. Alberola, L. Calvo, A. G. Ortega, M. L. Sábada, M. C. Sañudo, S. G. Granda, E. G. Rodriguez Heterocycles, 1999, 51, 2675; X. Wang, J. Tan, K. Grozinger Tetrahedron Lett. 2000, 41, 4713; A. R. Katritzky, M. Wang, S. Zhang, M. V. Voronkov J. Org. Chem., 2001, 66, 6787; and J. Bohrisch, M. Pätzel, C. Mügge, J. Liebscher Synthesis, 1991, 1153. Further guidance for synthesizing pyrazoles may be found in J. Elguero in Comprehensive Heterocyclic Chemistry II, A. R. Katritzky, C. W. Reees, E. F. V. Scriven., Eds.; Pergamon Press, Oxford, 1996; Vol. 3, p.1.

### EXAMPLES

The following examples are provided by way of illustration only and not by way of limitation. Those of skill in the art will readily recognize a variety of noncritical parameters that could be changed or modified to yield essentially similar results.

The prodrugs of TABLES 1-13 were synthesized according to the methods described below or illustrated in FIGS. 1-16. Melting points were obtained using an Electrothermal IA9100 series digital melting point apparatus. All Melting points are uncorrected. Elemental analysis was performed by Desert Analytics, Tuscon, Ariz. NMR spectra were obtained on a 300 MHz Varian Mercury system. Microwave reactions were carried out in the Personal Chemistry, SmithCreator microwave. LC-MS was performed on a Waters Micromass ZQ instrument with electrospray ionization. The HPLC component was a Waters Model 2690 Separation module coupled to a Waters Model 996 photodiode array detector at 254 nm wavelength. The specific LC-MS method used to analyze particular prodrugs, indicated for each prodrug in parentheses, are provided below:

### Method W

This method utilized a 2.1×250 mm 5 μM C-18 Altima reversed phase column (Alltech) with a flow rate of 0.25 mL/min and a gradient of 5-85% acetonitrile with water containing 0.1% trifluoroacetic acid over 36 min. The gradient then ramps to 100% acetonitrile over 0.5 min and continues at 100% acetonitrile for 3.5 min.

### Method X

This method utilized a 2.1×250 mm 5 μM C-18 Altima reversed phase column (Alltech) with a flow rate of 0.25 mL/min and a gradient of 5-85% acetonitrile with water containing 0.1% trifluoroacetic acid over 15 min. The gradient then ramps to 100% acetonitrile over 0.5 min and continues at 100% acetonitrile for 2.5 min.

### Method Y

This method utilized a 2.1×150 mm Agilent Zorbax 5 μM C-18 reversed phase column with a flow rate of 0.3 mL/min and a gradient of 10-100% acetonitrile with water containing 0.1% trifluoroacetic acid over 16 min, then continuing for 2 min with 100% acetonitrile.

### Method Y1

This method utilized a 2.1×150 mm Agilent Zorbax 5 μM C-18 reversed phase column with a flow rate of 0.3 mL/min and a gradient of 5-100% acetonitrile with water containing 0.05% formic acid over 15 min, then continuing for 5 min with 100% acetonitrile.

### Method Z

This method utilized a 2.1×5 mm Agilent Zorbax 5 μM C-18 reversed phase column with a flow rate of 0.5 mL/min and a gradient of 5-100% acetonitrile with water containing 0.1% trifluoroacetic acid over 8 min, then continuing for 2 min with 100% acetonitrile.

### Method A

LC-MS was performed on a Waters Micromass ZMD instrument with electrospray ionization. This method utilized a 2.1×5 mm Agilent Zorbax 5 μM C-18 reversed phase column with a flow rate of 0.3 mL/min and a gradient of 10-100% acetonitrile with water containing 0.05% formic acid over 10 min, then continuing for 8 min with 100% acetonitrile.

### Method B

This method utilized a 2.1×5 mm Agilent Zorbax 5 μM C-18 reversed phase column with a flow rate of 0.8 mL/min and a gradient of 5-95% acetonitrile.with water containing 0.05% formic acid over 5 min, then continuing for 2 min with 95% acetonitrile.

### Phosphonate Containing Prodrugs

Phosphonate containing compounds of the invention, which may be used as prodrugs, can be synthesized using the general synthetic schemes described in FIGS. 1 and 2. Syntheses of phosphonate containing prodrugs are provided below.

### Synthesis of 2,2-dichloro-2-(diethoxyphosphonyl)-N-[3-[3-(2,6-dichlorophenyl)-5-isoxazoyl]phenyl] acetamide

Reference may be made to FIG. 1 for illustration of the following synthesis descriptions. FIG. 3 shows an alternative synthetic scheme to prepare the final products. Diethylphosphonoacetic acid (5.6 g, 28.6 mmol) in methylene chloride (CH₂Cl₂, 50 mL) was treated with oxalyl chloride (3.7 mL, 42.9 mmol) followed by several drops of N,N-dimethylformamide (DMF). The reaction stirred at room temperature for 2 h and then the solvent was removed under vacuum to yield diethylphosphonoacetyl. Sulfuryl chloride (9.6 mL) was carefully added to the acid chloride, resulting in immediate evolution of gas. Then a catalytic amount of aluminum chloride was added. The mixture was heated at 80° C. for 3.5 h. The mixture was cooled and then distilled at approximately 1.3 mm Hg to give 2,2-dichloro-2-(diethoxyphosphonyl)acetyl chloride chloride (203, wherein X=Cl, R¹⁸=R¹⁹=Et) as a viscous colorless liquid (3.95 g, b.p. 95-110° C.).

3-Ethynylaniline (201, wherein W=CH; 1.64 g, 14 mmol) was dissolved in methylene chloride (10 mL) with triethylamine (Et₃N, 2.56 mL, 18.2 mmol). The solution was cooled to 4° C. under nitrogen and then a solution of 2,2-dichloro-2-(diethoxyphosphonyl)acetyl chloride (203, wherein X=Cl, R¹⁸=R¹⁹=Et; 3.95 g, 14 mmol) in methylene chloride (10 mL) was added dropwise. The reaction was allowed to warm to room temperature overnight with stirring. The reaction mixture was washed with 10% aqueous hydrochloric acid, water and saturated aqueous sodium bicarbonate. The organic phase was then dried over anhydrous sodium sulfate, filtered, and the filtrate concentrated under vacuum to afford the crude product, 1-[2′,2′-dichloro-2′-(diethoxyphosphonyl) acetylamino]-3-ethynyl benzene (205, wherein W=CH, X=Cl, R¹⁸=R¹⁹=Et), as a beige solid (4.11 g) which was used in the next step without further purification; MW=364 confirmed by LC-MS, tᵣ=14.31 min (Method Y) MH⁺=362-366.

1-[2′,2′-Dichloro-2′-(diethoxyphosphonyl)acetylamino]-3-ethynyl benzene (205, wherein W=CH, X=Cl, R¹⁸=R¹⁹=Et; 0.36 g, 1 mmol) was dissolved in anhydrous tetrahydrofuran (20 mL) and then treated with 2,6-dichloroN-hydroxybenzenecarboximidoyl chloride (207, wherein R²=R⁶=Cl; 0.23 g, 1 mmol) and triethylamine (0.26 mL, 1.3 mmol). After stirring at room temperature for 15 min, the reaction was heated at reflux for 4 h. The mixture was then cooled to room temperature and diluted with ethyl acetate. The organic solution was washed with brine and then dried over anhydrous sodium sulfate, filtered, and the filtrate concentrated under vacuum. The resulting residue was purified by flash column chromatography, on silica gel, eluting with 25% ethyl acetate in hexanes to give 2,2-dichloro-24diethoxyphosphonyl)-N-[3-[3-(2,6-dichlorophenyl)5-isoxazoyl]phenyl] acetamide (209, wherein W=CH, X=Cl, R¹⁸=R¹⁹=Et, R²=R⁶=Cl) as a white solid (103 mg); MW=552 confirmed by LC-MS, tᵣ=17.25 min (Method Y) MH⁺=550 554.

### Synthesis of 2,2-dichloro-2-(dihydroxyphosphonyl)-N-[3-[3-(2,6-dichlorophenyl)-5-isoxazoyl]phenyl] acetamide

2,2-Dichloro-2-(diethoxyphosphonyl)-N-[3-[3-(2,6-dichlorophenyl)-5-isoxazoyl]phenyl] acetamide (209, wherein W=CH, X=Cl, R¹⁸=R¹⁹=Et, R²=R⁶=Cl; 254 mg) was dissolved in anhydrous methylene chloride (1 mL). Then, trimethylsilyl bromide (1.0 mL, 7.6 mmol, Aldrich) was added. The reaction stirred for 27 h at room temperature. The volatiles were removed under vacuum to give a pale yellow foam which was dissolved in methanol:water (1:1) and shaken for 30 min. The mixture was filtered through Celite and the filter cake was washed with ethyl acetate. The filtrate was washed with brine, dried over anhydrous sodium sulfate, filtered and the filtrate concentrated under vacuum to give the 2,2-dichloro-24dihydroxyphosphonyl)-N-[3-[3-(2,6-dichlorophenyl)-5-isoxazoyl]phenyl] acetamide (211, wherein W=CH, X=Cl, R²=R⁶=Cl) as a pale yellow solid (59 mg); MW=496 confirmed by LC-MS, tᵣ=10.15 min (Method Y) MH⁺=494-500

The following prodrugs were made by similar methods as those described above and/or in FIGS. 1-3.

Cpd. 1: 2′-chloro-2′-(diethoxyphosphonyl)-N-[3-[3-(2,6-dichlorophenyl)-5-isoxazoyl]phenyl] acetamide; MW=516 confirmed by LC-MS, tᵣ=14.47 min (Method Y) MH⁺=514-518

Cpd. 2: 2′-chloro-2′-(diethoxyphosphonyl)-2-fluoro-N-[3-[3-(2,6-dichlorophenyl)-5-isoxazoyl]phenyl] acetamide; MW=534 confirmed by LC-MS, tᵣ=15.52 min (Method Y) MH⁺=534-537

Cpd. 3: 2′-(diethoxyphosphonyl)-2′,2′-difluoro-N-[3-[3-(2,6-dichlorophenyl)-5-isoxazoyl]phenyl] acetamide; MW=519 confirmed by LC-MS, tᵣ=15.27 min (Method Y) MH⁺=517-521

Cpd. 4: 2′,2′-dichloro-2′-(diisopropoxyphosphonyl)-N-[3-[3-(2,6-dichlorophenyl)-5-isoxazoyl]phenyl] acetamide; MW=580 confirmed by LC-MS, tᵣ=17.22 min (Method Y) MH⁺=578-582

Cpd. 5: 2′,2′-dichloro-2′-(diethoxyphosphonyl)-N-[3-[3-2-trifluoromethylphenyl)-5-isoxazoyl]phenyl] acetamide; MW=551 confirmed by LC-MS, tᵣ=15.67 min (Method Y)

Cpd. 6: 2′,2′-dichloro-2′-(diethoxyphosphonyl)-N-[3-[3-(2-fluoro-6-trifluoromethylphenyl)-5-isoxazoyl]phenyl] acetamide; MW=569 confirmed by LC-MS, tᵣ=15.64 min (Method Y) MH⁺=567-571

### Ester Containing Prodrugs

Ester containing compounds of the invention, which may be used as prodrugs, can be synthesized using the general synthetic schemes described in FIGS. 5 and 6. Syntheses of phosphonate containing prodrugs are provided below.

### Synthesis of 2′,2′-dichloro-2′-(t-butoxycarbonyl)-N-[3-[3-(2,6-dichlorophenyl)-5-isoxazoyl]phenyl]acetamide

Reference may be made to FIG. 4 for illustration of the following synthesis description. Mono-t-butyl malonate (Aldrich, 10 g, 62.5 mmol) was dissolved in methylene chloride (40 mL) and treated with oxalyl chloride (8.8 mL, 84 mmol) followed by several drops of N,N-dimethylformamide. After 80 min the reaction was concentrated under vacuum to yield the malonyl acid chloride t-butyl ester. The acid chloride was treated with sulfuryl chloride (23 mL) and a catalytic amount of aluminum chloride. The mixture was heated for 3.5 h at 80° C., whereupon NMR analysis showed complete consumption of starting material. The reaction was distilled at 1.3 mm Hg to give 5.73 g of 2-(t-butoxycarbonylI2,2-dichloroacetyl chloride (603, wherein R¹⁶=t-butyl; b.p. 52-70° C.).

3-Ethynylaniline (601, wherein W=CH; 2.72 g, 23.3 mmol) was dissolved in methylene chloride (10 mL) with triethylamine (4.25 mL, 30.3 mmol). The solution was cooled to 4° C. under nitrogen and then a solution of 24t-butoxycarbonylS2,2-dichloroacetyl chloride (603, wherein R¹⁶=t-butyl; 5.73 g, 23.3 mmol) in methylene chloride (20 mL) was added dropwise. The reaction was allowed to warm to room temperature overnight. The reaction mixture was washed with saturated aqueous sodium bicarbonate and brine. The reaction was then dried over anhydrous sodium sulfate, filtered, and the filtrate concentrated under vacuum. The crude product was purified by flash column chromatography, on silica gel, eluting with 20% ethyl acetate in hexanes to afford the product, 1-[2′-(t-butoxycarbonyl)-2′,2′-dichloroacetylaminol-3-ethynylbenzene (605, wherein W=CH, X=Cl, R¹⁶=t-butyl), as an amber oil. Crystallization from hexanes and ethyl acetate gave brown crystals (3.07 g).

1-[2′-(t-Butoxycarbonyl)-2′,2′-dichloroacetylamino]-3-ethynylbenzene (605, wherein W=CH, X=Cl, R¹⁶=t-butyl; 0.70 g, 2.13 mmol) was dissolved in anhydrous tetrahydrofuran (20 mL) and then treated with 2,6-dichloro-N-hydroxybenzenecarboximidoyl chloride (607, wherein R²=R⁶=Cl; 0.48 g, 2.13 mmol) and triethylamine (0.40 mL, 2.8 mmol) to generate the corresponding nitrile oxide in situ. After stirring at room temperature for 15 min, the reaction was heated at reflux for 5.5 h, during which time a [3+2] cycloaddition reaction occurred. The mixture was then cooled to room temperature and diluted with ethyl acetate. The organic solution was washed with brine and then dried over anhydrous sodium sulfate, filtered, and the filtrate concentrated under vacuum. The resulting residue was purified by flash column chromatography, on silica gel, eluting with 20% ethyl acetate in hexanes to give 2′,2′-dichloro-2′-(t-butoxycarbonyl)-N-[3-[3-(2,6-dichlorophenyl5-isoxazoyl]phenyl]acetamide (609, wherein W=CH, X=Cl, R²=R⁶=Cl, R¹⁶=t-butyl) as a white solid (426 mg); MW=516 confirmed by LC-MS, tᵣ=15.40 min (Method A) MH⁺=514-518.

### Synthesis of 2′,2′-dichloro-2′-(isopropoxycarbonyl)-N-[3-[3-(2,6-dichlorophenyl)-5-isoxazoyl]phenyl] acetamide

Reference may be made to FIG. 6 for illustration of the following synthesis description. Meldrum's acid (8.0 g, 55.6 mmol) and isopropanol (3.34 g, 55.6 mmol) were heated at reflux in acetonitrile (CH₃N, ACN, 30 mL) for 24 h. The reaction was concentrated under vacuum to yield mono-isopropyl malonate, which was used directly for the next step.

The mono-isopropyl malonate, prepared above (55.6 mmol) was dissolved in methylene chloride (40 mL) and then treated with oxalyl chloride (7.77 mL, 89 mmol) and several drops of N,N-dimethylformamide. After 1.5 h the reaction was concentrated under vacuum and then treated with sulfuryl chloride (20 mL) followed by a catalytic amount of aluminum chloride. The reaction was heated at 80° C. for 4 h, where upon NMR revealed the starting material was completely consumed. The mixture was cooled and then distilled at approximately 1.3 mm Hg to give 2-(isopropoxycarbonyl)-2,2-dichloroacetyl chloride 803 as a clear liquid (5.67 g. b.p. 55° C.).

3-Ethynylaniline 801 (2.86 g, 24.4 mmol) was dissolved in methylene chloride (40 mL) with triethylamine (4.46 mL, 31.7 mmol). The solution was cooled to 4° C. under nitrogen and then a solution of 2-(isopropoxycarbonyl)-2,2-dichloroacetyl chloride 803 (5.67 g, 24.4 mmol) in methylene chloride (20 mL) was added dropwise. The reaction was allowed to warm to room temperature overnight. The reaction mixture was washed with 10% aqueous hydrochloric acid, water and saturated aqueous sodium bicarbonate. The reaction was then dried over anhydrous sodium sulfate, filtered, and the filtrate concentrated under vacuum. The crude product was purified by flash column chromatography, on silica gel, eluting with 20% ethyl acetate in hexanes to afford the product, 1-[2′-(isopropoxycarbonyl)-2′,2′-dichloroacetylamino]-3-ethynylbenzene 805, as an oil which solidified upon standing; MW=314 confirmed by LC-MS, tᵣ=16.27 min (Method A) MH⁺=312-316.

1-[2′-(Isopropoxycarbonyl)-2′,2′-dichloroacetylaminol-3-ethynylbenzene 805 (0.27 g, 0.86 mmol) was dissolved in anhydrous tetrahydrofuran (20 mL) and treated with 2,6-dichloro-N-hydroxybenzenecarboximidoyl chloride 807 (0.19 g, 0.86 mmol) and triethylamine (0.15 mL) to generate the corresponding nitrile oxide in situ. The mixture was stirred at room temperature for 30 min, then heated to reflux for 5 h during which time a [3+2] cycloaddition reaction occurred. The reaction was cooled, diluted with ethyl acetate and washed with brine. The organic layer was dried over anhydrous sodium sulfate, filtered and the filtrate concentrated under vacuum. The crude product was purified by flash column chromatography, on silica gel, eluting with 20% ethyl acetate in hexanes to give 2′,2′-dichloro-2′-(isopropoxycarbonyl)-N-[3-[3-(2,6-dichlorophenyl)-5-isoxazoyl]phenyl] acetamide 809 as a white solid; MW=502 confirmed by LC-MS, tᵣ=18.82 min (Method Y) MH⁺=500-504.

The following prodrugs were made by similar methods as those described above and/or in FIGS. 4-7. FIGS. 4 and 5 show alternative synthetic approaches to prepare prodrugs of the invention. Additionaly, the teachings of Page, P. C. B.; Moore, J. P. G.; Mansfield, I.; McKenzie, M. J.; Bowler, W. B.; Gallagher, J. A. Tetrahedron. 57, 1837 (2001) provide various methods to prepare starting materials for the prodrugs.

Cpd. 8: 2′,2′-dichloro-2′-(1S-ethoxycarbonyl-1-methylmethyleneoxycarbonyl)-N-[3-[3-(2,6-dichlorophenyl)-5-isoxazoyl]phenyl] acetamide; MW=560 confirmed by LC-MS, tᵣ=16.92 min (Method Y) MH⁺=558-56

Cpd. 9: 2′,2′-dichloro-2′-[(1S)-endo-borneyloxycarbonyl]-N-[3-[3-(2,6-dichlorophenyl)-5-isoxazoyl]phenyl] acetamide; MW=593 confirmed by LC-MS, tᵣ=14.62 min (Method Y) MH⁺=591-595

Cpd. 1008: 2′,2′-dichloro-2′-(1-adamantyloxycarbonyl)-N-[3-[3-(2,6-dichlorophenyl)-5-isoxazoyl]phenyl] acetamide; MW=594 confirmed by LC-MS, tᵣ=14.77 min (Method A) MH⁺=592-596

Cpd. 11: 2′,2′-dichloro-2′-[(1R, 2S, 5Rkmenthyloxycarbonyl]-N-[3-[3-(2,6-dichlorophenyl)-5-isoxazoyl]phenyl] acetamide; MW=598 confirmed by LC-MS, tᵣ=15.12 min (Method A) MH⁺=596-600

Cpd. 12: 2′,2′-dichloro-2′-(sec-butoxycarbonyl)-N-[3-[3-(2,6-dichlorophenyl)-5-isoxazoyl]phenyl] acetamide; MW=516 confirmed by LC-MS, tᵣ=15.78 min (Method Y) MH⁺=514-518

Cpd. 13: 2′,2′-dichloro-2′-(cyclohexyloxycarbony)-N-[3-[3-(2,6-dichlorophenyl)-5-isoxazoyl]phenyl] acetamide; MW=542 confirmed by LC-MS, tᵣ=16.20 min (Method A) MH⁺=540-544

Cpd. 14: (2′,2′-dichloro-2′-(neopentyloxycarbonyl)-N-[3-[3-(2,6-dichlorophenyl)-5-isoxazoyllphenyl] acetamide; MW=530 confirmed by LC-MS, tᵣ=15.78 min (Method A) MH⁺=528-532

Cpd. 15: (2′,2′-dichloro-2′-(t-butoxycarbonyl)-N-[3-[3-(2,6-dichlorophenyl)-5-isoxazoyl]phenyl] acetamide; MW=516 confirmed by LC-MS, tᵣ=15.40 min (Method A) MH⁺=514-518

Cpd. 16: 2′,2′-dichloro-2′-(isopropoxycarbonyl)-N-[3-[3-(2,6-dichlorophenyl)-5-isoxazoyl]phenyl] acetamide; MW=502 confirmed by LC-MS, tᵣ=19.11 min (Method Y) MH⁺=500-504

Cpd. 17: 2′,2′-dichloro-2′-(benzyloxycarbonyl)-N-[3-[3-(2,6-dichlorophenyl)-5-isoxazoyl]phenyl] acetamide; MW=550 confirmed by LC-MS, tᵣ=19.23 min (Method Y) MH⁺=548-552

Cpd. 18: 2′,2′-dichloro-2′-(methoxycarbonyl)-N-[2-[3-(2,6-dichlorophenyl)-5-isoxazoyl]phenyl] acetamide; MW=474 confirmed by LC-MS, tᵣ=17.00 min (Method Y) MH⁺=472-476

Cpd. 505: 2′,2′-dichloro-2′-(t-butoxycarbonyl-[2-[3-(2,6-dichlorophenylS5-isoxazoyl]-(4-pyridyl)] acetamide; MW=518 confirmed by LC-MS, tᵣ=17.04 min (Method Y) MH⁺=516-520

Cpd. 25: 2′,2′-dichloro-2′-(t-butoxycarbonyl)-N-[3-[3-(2-chloro-6-methoxyphenyl 5-isoxazoyl]phenyl] acetamide; MW=511 confirmed by LC-MS, tᵣ=17.02 min (Method Y) MH⁺=509-513

Cpd. 26: 2′,2′-dichloro-2′-(methoxycarbonyl)-N-[3-[3-(2-cyclopropyl-6-trifluoromethylphenyl]5-isoxazoyl]phenyl] acetamide; MW=513 confirmed by LC-MS, tᵣ=18.06 min (Method Y) MH⁺=511-515

Cpd. 27: 2′,2′-dichloro-2′-(methoxycarbonyl)-N-[3-[3-(2-chloro-6-methoxyphenyl]5-isoxazoyl]phenyl] acetamide; MW=526 confirmed by LC-MS, tᵣ=16.70 min (Method Y) MH⁺=524-528

Cpd. 28: 2′,2′-dichloro-2′-(methoxycarbonyl)-N-[3-[3-(6chloro-2-dimethylamino-3-pyridine-1-yl)-5-isoxazoyl]phenyl] acetamide; MW=483 confirmed by LC-MS, tᵣ=14.49 min (Method Y) MH⁺=481-485

Cpd. 29: 2′,2′-dichloro-2′-(methoxycarbonyl)-N-[3-[3-(2-trifluoromethylphenyl)-5-isoxazoyl]phenyl] acetamide; MW=473 confirmed by LC-MS, tᵣ=15.88 min (Method Y) MH⁺=471-475

Cpd. 30: 2′,2′-dichloro-2′-(methoxycarbonyl)-N-[3-[3-(2-fluoro-6-trifluoromethylphenyl)-5-isoxazoyl]phenyl] acetamide; MW=491 confirmed by LC-MS, tᵣ=17.21 min (Method Y) MH⁺=489-493

### Dioxolenone Containing Prodrugs

Dioxolenone containing compounds of the invention, which may be used as prodrugs, can be synthesized using general procedures similar to the specific examples provided below.

### Synthesis of 2,2-Dichloro-N-[3′-[3′-(2′,6′-dichlorophenyl)-5′-isoxazoyl]]-N-[(5-methyl-1,3-dioxolene-2-one-4-yl)methylene] Acetamide

To a solution of 4,5-dimethyl-1,3-dioxolene-2-one (TCl, 10 g, 88 mmol) and N-bromosuccinimide (Fluka, 15.69 g, 88 mmol) in carbon tetrachloride (250 mL) was added benzoyl peroxide (Acros, 500 mg, 2.1 mmol). The reaction mixture was then refluxed for 2.5 h after which time the volatiles were evaporated under vacuum. The resulting residue was triturated with some carbon tetrachloride, filtered and the solid cake was washed with carbon tetrachloride. The filtrate volatiles were removed under vacuum and the yellow oily residue was distilled under vacuum (2-5 torr) to give 4-bromomethyl-5-methyl-1,3-dioxolene-2-one 903 (8.35 g, b.p. 94-98° C., 49%) as a pale yellow oil. ¹H NMR (CDCl₃) δ 4.21 (s, 2H), 2.17 (s, 3H).

A mixture of 3-(2,6-dichlorophenyl)-5-(3-aminophenyl)isoxazole 401 (117 mg, 0.4 mmol), 4-bromomethyl-5-methyl-1,3-dioxol-2-one 903 (Sakamoto, F. et al. Chem. Pharm. Bull. 1984, 32, 2241) (85 mg, 0.44 mmol), sodium bicarbonate (40 mg, 0.5 mmol) in acetonitrile (5 mL) was allowed to reflux for 3 h. The reaction mixture was then concentrated under vacuum, and the resulting residue was partitioned between ethyl acetate (30 mL) and brine (30 mL). The separated organic layer was washed with brine (2×20 mL), dried over anhydrous magnesium sulfate, filtered and the filtrate concentrated under vacuum. Column chromatography (neat methylene chloride) provided N-[3′-[3′-(2′,6′-dichlorophenyl)-5′-isoxazoyl]]N-[(5-methyl-1,3-dioxolene-2-one-4-yl)methylene]aniline 905 (65 mg, 41%). ¹H NMR (CDCl₃) δ 7.47 (d, J=1.7 Hz, 1H), 7.45 (d, J=0.6 Hz, 1H), 7.38 (d, J=6.9 Hz, 1H), 7.34 (t, J=6.9 Hz, 1H), 7.25 (dt, J=7.7, 1.4 Hz, 1H), 7.16 (t, J=2.0 Hz, 1H), 6.74 (dq, J=8.0, 1.1 Hz, 1H), 6.61 (s, 1H), 4.22 (d, J=0.6 Hz, 1H), 2.23 (s, 3H). MS (m/z): 417 (MH⁺).

To an ice-cold solution of N-[3′-[3′-(2′,6′-dichlorophenyl)-5′-isoxazoyl]]-N-[(5-methyl-1,3-dioxolene-2-one-4yl)methylene]aniline 905 (65 mg, 0.2 mmol) in methylene chloride (2 mL) was added, dropwise, triethylamine (25 μl, 18 mg, 0.2 mmol), followed by dichloroacetyl chloride (17 μL, 26 mg, 0.2 mmol). The resulting reaction mixture was allowed to warm up to room temperature over 90 min. Methylene chloride (10 mL) and saturated ammonium chloride solution were then added to the reaction mixture. The layers were separated and the organic layer was washed with saturated ammonium chloride solution (1×20 mL) and dried over anhydrous magnesium sulfate, filtered and the filtrate concentrated under vacuum. Column chromatography, on silica gel, eluting with neat methylene chloride, provided 2,2-dichloro-N-[3′-[3′-(2′,6′-dichlorophenyl)-5′-isoxazoyl]phenyl]N-[(5-methyl-1,3-dioxolene-2-one4-yl)methylene] acetamide 907 (51 mg, 62%) as a colorless oil. ¹H NMR (CDCl₃) 67 8.00 (dq, J=8.0, 1.1 Hz, 1H), 7.84 (t, J=1.7 Hz, 1H), 7.70 (t, J=7.8 Hz, 1H), 7.50-7.37 (m, 4H), 6.76 (s, 1H), 5.91 (s, 1H), 4.72 (br s, 2H), 2.25 (s, 3H). MS (m/z): 527 (MH⁺).

### Synthesis of 2,2-Dichloro-N-[3′-[3′-(2′,6′-dichlorophenyl)-isoxazol-5′-yl]]phenyl]-N-[(5-t-butyl-1,3-dioxolene-2-one-4-yl)methylene] Acetamide

Reference may be made to FIG. 9 for illustration of the following synthesis description. By the procedure of Moftet [Moftet, C. et al. J. Org. Chem. 1999, 64, 1380] A solution of ethyl 4,4-dimethyl-3-oxo-pentanoate (40 mL, 38.7 g, 225 mmol) and benzyl alcohol (28 mL, 29.3 g, 270 mmol) in toluene (1.5 L) was heated so the toluene refluxed halfway up the refluxing tube (oil bath temperature 120° C.) overnight. After being cooled down to room temperature, the volatiles were evaporated under vacuum and the resulting crude oil was purified by column chromatography, on silica gel, eluting with neat methylene chloride. The desired product, benzyl 4,4-dimethyl-3-oxo-pentanoate, was obtained as a colorless oil (39.5 g, 75%). ¹H NMR (CDCl₃) δ 7.39-7.37 (m, 5H), 5.20 (s, 2H), 3.64 (d, 2H), 1.20 (s, 9H).

A cold solution (ice bath) of benzyl 4,4-dimethyl-3-oxo-pentanoate (14.23 g, 61 mmol) in acetonitrile (500 mL) was added 4-acetamidobenzenesulfonyl azide (14.60 g, 61 mmol) and triethylamine (25.4 mL, 18.4 g, 182 mmol). The yellow reaction mixture was allowed to stir at 0° C. for 30 min, then at room temperature overnight. The reaction mixture was concentrated under vacuum and the resulting solid white residue was triturated with ethyl ether/hexanes (2:1, 3×200 mL) and filtered. The filtrate was concentrated under vacuum and purified by column chromatography, on silica gel, eluting with 15% ethyl acetate/hexanes, to provide benzyl 2-diazo-4,4-dimethyl-3-oxo-pentanoate (15.66 g, 99%). ¹H NMR (CDCl₃) δ 7.39-7.37 (m, 5H), 5.27 (s, 2H), 1.33 (s, 9H).

To a solution of benzyl 2-diazo4,4-dimethyl-3-oxo-pentanoate (15.66 g, 60.2 mmol) in tetrahydrofuran/water (2:1, 375 mL) was added rhodium (II) acetate dimer (Rh₂(OAc)₄, Aldrich, 525 mg, 1.2 mmol) in three portions (3×175 mg every 2 hrs) and the resultant green solution was heated at reflux overnight. The reaction mixture was concentrated under vacuum and the residue extracted with ethyl acetate (2×300 mL). The combined extracts were washed with brine (2×125 mL), dried over anhydrous magnesium sulfate, filtered and the filtrate concentrated under vacuum to give 14.87 g of the crude yellow oil. Column chromatography, on silica gel, eluting with 10% ethyl acetate/hexanes, provided benzyl 4,4-dimethyl-2-hydroxy-3-oxo-pentanoate (7.5 g, 50%). ¹H NMR (CDCl₃) δ 7.39-7.36 (m, 5H), 5.23 (d, J=2.7 Hz, 2H), 5.09 (d, J=8.3 Hz, 2H), 3.97 (d, J=8.3 Hz, 2H), 1.20 (s, 9H).

By the procedure of Sun [Sun, C. -Q. et al. Tet. Lett. 2002, 43, 1161], an ice-cold solution of benzyl 4,4-dimethyl-2-hydroxy-3-oxo-pentanoate (3.8 g, 15.2 mmol) in tetrahydrofuran (75 mL) was treated with carbonyldiimidazole (CDI, 4.93 g, 30.4 mmol) followed by diisopropylethylamine (DIEA, 110 μL, 81 mg, 0.6 mmol). The reaction mixture was allowed to stir overnight without removal of the cold bath. The resulting reaction mixture was concentrated under vacuum and the residue partitioned between ethyl acetate (100 mL) and 5% potassium hydrogen sulfate solution (100 mL). The separated organic layer was washed with 5% potassium hydrogen sulfate solution (1×100 mL), water (2×100 mL), brine (2×100 mL), dried over anhydrous magnesium sulfate, filtered and the filtrate concentrated under vacuum to give an oily residue. Column chromatography on silica gel, eluting with 10% ethyl acetate/hexanes, provided benzyl 5-t-butyl-1,3-dioxolene-2-one4arboxylate (2.8 g, 68%). ¹H NMR (CDCl₃) δ 7.42-7.41 (m, 5H), 5.34 (s, 2H), 1.42 (s, 9H).

By the procedure of Sun [Sun, C. -Q. etal. Tet. Lett. 2002, 43, 1161], a mixture of benzyl 5-t-butyl-1,3-dioxolene-2-one4-carboxylate (6.0 g, 21.7 mmol) and Pd(OH)₂/C (270 mg) in absolute ethanol (150 mL) was allowed to mix under hydrogen atmosphere at 18 psi for 1 h. The resulting reaction mixture was filtered through a bed of Celite and the filtrate was concentrated under vacuum to give 5-t-butyl-1,3-dioxolene-2-one4-carboxylic acid (3.8 g, 94%). ¹H NMR (CDCl₃) δ 1.45 (s, 9H).

By the procedure of Sun [Sun, C. -Q. et al. Tet. Lett. 2002, 43, 1161], an ice-cold solution of 5-t-butyl-1,3-dioxolene-2-one-4-carboxylic acid (1.2 g, 6.4 mmol) and N,N-dimethylformamide (70 μL) in methylene chloride (30 mL) was stirred and oxalyl chloride (630 μL, 0.92 g, 7.2 mmol) was added dropwise. The reaction mixture was allowed to stir at 0° C. for 30 min and at room temperature for 45 min before being concentrated and dried under vacuum. The residue containing carbonyl chloride was dissolved in methylene chloride (30 mL), cooled to −78° C. and treated with a solution of tetra-n-butylammonium borohydride (1.83 g, 7.1 mmol) in methylene chloride (30 mL). The reaction mixture was allowed to stir at −78° C. for 1 h. and quenched with 0.1 N hydrochloric acid solution (20 mL). The reaction mixture was allowed to warm up to room temperature after which the volatiles were removed under vacuum. The residue was partitioned between ethyl acetate (60 mL) and water (30 mL). The separated aqueous layer was saturated with sodium chloride and extracted with ethyl acetate (2×50 mL). The combined organic extracts were washed with brine (1×100 mL), dried over anhydrous magnesium sulfate, filtered and the filtrate concentrated under vacuum to give an oily residue. Column chromatography, on silica gel, eluting with 40% ethyl acetate/hexanes, provided 5-t-butyl-4-hydroxymethyl-1,3-dioxolene-2-one (0.77 g, 69%). ¹H NMR (CDCl₃) δ 4.53 (d, J=4.1 Hz, 2H), 1.34 (s, 9H).

By the procedure of Sun [Sun, C. -Q. et al. Tet. Lett. 2002, 43, 1161], an ice-cold solution of 5-t-butyl-4-hydroxymethyl-1,3-dioxolene-2-one (0.77 g, 4.5 mmol) and methylene chloride (25 mL) was stirred and carbon tetrabromide (1.78 g, 5.4 mmol) and triphenylphosphine (1.30 g, 5.0 mmol) were added slowly. The yellow reaction mixture was allowed to stir at 0° C. for 35 min before being concentrated under vacuum. The yellow oily residue was adsorbed onto Celite and purified on a silica gel column, eluting with 10% ethyl acetate/hexanes, to provide 4-bromomethyl-5-t-butyl-1,3-dioxolene-2-one 1003 (0.83 g, 79%). ¹H NMR (CDCl₃) δ 4.32 (s, 2H), 1.34 (s, 9H).

A mixture of 342,6-dichlorophenyl543-aminophenyl)isoxazole 401 (102 mg, 0.3 mmol), 4-bromomethyl-5-tbutyl-1,3-dioxolene-2-one 1003 (Sakamoto, F. et al. Chem. Pharm. Bull. 1984, 32, 2241) (87 mg, 0.4 mmol) and sodium bicarbonate (31 mg, 0.4 mmol) in acetonitrile (5 mL) was allowed to reflux under nitrogen atmosphere for 3 h. The reaction mixture was then concentrated under vacuum, and the resulting off-white residue was chromatographed, on silica gel, eluting with neat methylene chloride, to provide N-[3′-[3′-(2′,6′-dichlorophenyl)-5′-isoxazolyl]phenyl]-N-[5-t-butyl-1,3-dioxolene-2-one4-yl)methylene]aniline 1005 (125 mg, 81%). ¹H NMR (CDCl₃) δ 7.47 (d, J=1.7 Hz, 1H), 7.45 (d, J=0.6 Hz, 1H), 7.39-7.32 (m, 2H), 7.25 (dt, J=6.6, 1.1 Hz, 1H), 7.19 (t, J=1.8 Hz, 1H), 6.79 (ddd, J=8.0, 2.5, 1.1 Hz, 1H), 6.61 (s, 1H), 4.31 (s, 2H), 1.38 (s, 9H). MS (m/z): 459 (MH⁺) confirmed by LC-MS, tᵣ=16.98 min (Method Y).

To an ice-cold solution of N-[3′-[3′-(2′,6′-dichlorophenyl)-5′-isoxazolyl]phenyl]-N-[5-t-butyl-1,3-dioxolene-2-one-yl)methylene]aniline 1005 (120 mg, 0.3 mmol) in dichloromethane (3 mL) was added dropwise diisopropylethylamine (55 μl, 41 mg, 0.3 mmol) followed by dichloroacetyl chloride (28 ρL, 43 mg, 0.3 mmol). The reaction mixture was allowed to warm up to room temperature under nitrogen atmosphere over 2.5 h. Since the starting aniline was still present, additional diisopropylethylamine (50 μL, 37 mg, 0.3 mmol) and dichloroacetyl chloride (15 μL, 23 mg, 0.16 mmol) were added and the reaction mixture was allowed to stir overnight. Methylene chloride (20 mL) and saturated ammonium chloride solution (20 mL) were then added to the reaction mixture. The layers were separated and the organic layer was washed with saturated ammonium chloride solution (1×20 mL), brine (1×20 mL), dried over anhydrous magnesium sulfate, filtered and the filtrate concentrated under vacuum. Column chromatography, on silica gel, eluting with neat methylene chloride provided 2,2-dichloro-N-[3′-[3′-(2′,6′-dichlorophenyl)-5′-isoxazoyl]phenyl]-N-[(5-t-butyl-1,3-dioxolene-2-one4yl)methylene]acetamide 1007 (108 mg, 73%). ¹H NMR (CDCl₃) δ 8.00 (dt, J=8.0, 1.1 Hz, 1H), 7.82 (t, J=187 Hz, 1H), 7.69 (t, J=7.9 Hz, 1H), 7.50-7.36 (m, 4H), 6.74 (s, 1H), 5.88 (s, 1H), 5.13-4.72 (br d, 2H), 1.26 (s, 9H). MS (m/z): 569 (MH⁺) confirmed by LC-MS, tᵣ=16.88 min (Method Y).

The following prodrugs were made by similar methods as those described above. FIG. 10 is an alternative synthesis to prepare prodrugs on the invention.

Cpd. 31. 2,2-dichloro-N-[3′-[3′-(2′-chloro-6′-methoxyphenyl)-5′-isoxazoyl]phenyl]-N-[(5-methyl-1,3-dioxolene-2-one-4-yl)methylene] acetamide; MW=524 confirmed by LC-MS, tᵣ=15.00 min (Method Y) MH⁺=522-526

Cpd. 32. 2,2-dichloro-N-[3′-[3′-(2′,6′-dichloro-3′-pyridine-1-yl)-5′-isoxazoyl]phenyl]-N-[(5-methyl-1,3-dioxolene-2-one4-yl)methylene] acetamide; MW=527 confirmed by LC-MS, tᵣ=14.25 min (Method Y) MH⁺=525-529

Cpd. 33. 2,2-dichloro-N-[3′-[3′-(6′-chloro-2′-dimethylamino-3′-pyridine-1-yl)-5′-isoxazoyl]phenyl]-N-[(5-methyl-1,3-dioxolene-2-one4-yl)methylene] acetamide; MW=537 confirmed by LC-MS, tᵣ=13.00 min (Method Y) MH⁺=535-539

Cpd. 34. 2,2-dichloro-N-[3′-[3′-(2′,6′-dichlorophenyl)-5′-isoxazoyl]phenyl]-N-[(5-isopropyl-1,3-dioxolene-2-one4-yl)methylene] acetamide; MW=556 confirmed by LC-MS, tᵣ=16.49 min (Method Y) MH⁺=554-558

Cpd. 35. 2,2-dichloro-N-[3′-[3′-(6′-chloro-2′-dimethylamino-3′-pyridine-1-yl)-5′-isoxazoyl]phenyl]-N-[(5-isopropyl-1,3-dioxolene-2-one4-yl)methylene] acetamide; MW=566 confirmed by LC-MS, tᵣ=7.18 min (Method Z) MH⁺=564-568

Cpd. 36. 2,2-dichloro-N-[3′-[3′-(2′-chloro-6′-tbutoxycarbonylphenyl)-5′-isoxazoyl]phenyl]-N-[(5-isopropyl-1,3-dioxolene-2-one4-yl)methylene] acetamide; MW=622 confirmed by LC-MS, tᵣ=18.50 min (Method Y) MH⁺=620-624

Cpd. 37. 2,2-dichloro-N-[3′-[3′-(2′-chloro-6′-methoxyphenyl)-5′-isoxazoyl]phenyl]-N-[(5-isopropyl-1,3-dioxolene-2-one-4-yl)methylene] acetamide; MW=553 confirmed by LC-MS, tᵣ=16.46 min (Method Y) MH⁺=551-554

Cpd. 38. 2,2-dichloro-N-[3′-[3′-(2′,6′-dichlorophenyl)-5′-isoxazoyl]phenyl]-N-[(5-n-propyl-1,3-dioxolene-2-one-4-yl)methylene] acetamide; MW=556 confirmed by LC-MS, tᵣ=12.45 min (Method X) MH⁺=554-558

Cpd. 39. 2,2-dichloro-N-[3′-[3′-(2′,6′-dichlorophenyl)-5′-isoxazoyl]phenyl]-N-[(5-cyclohexyl-1,3-dioxolene-2-one4-yl)methylene] acetamide; MW=595 confirmed by LC-MS, tᵣ=13.28 min (Method X) MH⁺=593-597

Cpd. 40. 2,2-dichloro-N-[3′-[3′-(2′,6′-dichlorophenyl)-5′-isoxazoyllphenyl]-N-[(5-ethyl-1,3-dioxolene-2-one4-yl)methylene] acetamide; MW=542 confirmed by LC-MS, tᵣ=12.18 min (Method X) MH⁺=540-544

Cpd. 41. 2,2-dichloro-N-[2′-[3′-(2′,6′-dichlorophenyl)-5′-isoxazoyl](4-pyridyl)]-N-[(5-methyl-1,3-dioxolene-2-one4-yl)methylene] acetamide

Cpd. 42. 2,2-dichloro-N-[2′-[3′-(2′,6′-dichlorophenyl)-5′-isoxazoyl](4-pyridyl)]N-[(5-i-propyl-1,3-dioxolene-2-one4-yl)methylene] acetamide

Cpd. 1107.2,2-dichloro-N-[2′-[3′-(2′,6′-dichlorophenyl)-5′-isoxazoyl](4-pyridyl)]-N-[(5-t-butyl-1,3-dioxolene-2-one4-yl)methylene] acetamide

Cpd. 43. 2,2-dichloro-N-[2′-[3′-(2′,6′-dichlorophenyl)-5′-isoxazoyl](4pyridyl)]N-[(5-n-pentyl-1,3-dioxolene-2-one4yl)methylene] acetamide

Cpd. 44. 2,2-dichloro-N-[3′-[3-(2′,6′-dichlorophenyl)-5′-isoxazoyllphenyl]N-[(5-ethoxycarbonyl-1,3-dioxolene-2-one-4-yl)methylene] acetamide; MW=586 confirmed by LC-MS, tᵣ=12.10 min (Method Y) MH⁺=584-588

### Synthesis of 2.2-Dichloro-N-(3-(3-(2.6-dichlorophenyl)isoxazol-5-yl)phenyl)-N-(2-(Dhenylsulfonyl)ethyl)acetamide (See FIG. 23) (Cpd. 1045)

### Synthesis of 3-Ethynyl-N-(2-(phenylsulfonyl)ethyl)benzenamine

3-Ethynyl aniline (2.53 g, 21.6 mmol) and phenyl vinyl sulfone (4.36 g, 26 mmol) were heated at reflux in ethanol (50 mL) for 136 h. The solvent was removed under reduced pressure and the residue was partitioned between ethyl acetate and brine. The organic layer was separated and dried over anhydrous sodium sulfate, filtered and concentrated under reduced pressure. The resulting residue was purified by flash column chromatography on silica gel, eluting with 15:85 ethyl acetate:hexanes to provide 3-ethynyl-N-(2-(phenylsulfonyl)ethyl)benzenamine (1.21 g) as a brown solid. ¹H NMR (CDCl₃) δ 7.91 (m, 2H), 7.90-7.54 (m, 3H), 7.10 (t, 1H), 6.87 (d, 1H), 6.61 (br s, 1H), 6.53 (m, 1H) , 3.59 (t, 2H), 3.37 (t, 2H), 3.02 ppm (s, 1H). MW=285 confirmed by LC-MS, tᵣ=12.72 min (Method Y) MH⁺=286.

### Synthesis of 2,2-Dichloro-N-(3-ethynylphenyl)-N-(2-(phenylsulfonyl)ethyl)acetamide

3-Ethynyl-N-(2-(phenylsulfonyl)ethyl)benzenamine (1.21 g, 4.2 mmol) was dissolved in anhydrous methylene chloride (25 mL) with triethylamine (0.78 mL, 5.5 mmol). The mixture was cooled in an ice-bath under nitrogen, then a solution of dichloroacetyl chloride (0.45 mL, 4.6 mmol) in anhydrous dichloromethane (5 mL) was added dropwise. After the addition was completed the ice-bath was removed and the mixture was stirred at room temperature overnight. The reaction mixture was diluted with methylene chloride and then washed successively with water, 10% hydrochloric acid and saturated sodium bicarbonate solution. The organic solution was dried over anhydrous sodium sulfate, filtered and concentrated under reduced pressure. The resulting residue was purified by flash column chromatography on silica gel, eluting with 30:70 ethyl acetate:hexanes to give 2,2-dichloro-N-(3-ethynylphenyl)-N-(2phenylsulfonyl)ethyl)acetamide (120 mg) as a yellow oil. ¹H NMR (CDCl₃): 7.90 (m, 2H), 7.68 (m, 1H), 7.58 (m, 2H), 7.43 (t, 1H), 7.33 (br s, 1H), 7.26 (m, 1H), 5.74 (s, 1H), 4.04 (t, 2H), 3.42 (t, 2H), 3.22 ppm (s, 1H). MW=396 confirmed by LC-MS, tᵣ=13.61 min (Method Y) MH⁺=394-398.

### Synthesis of 2,2-Dichloro-N-(3-(3-(2,6-dichlorophenyl)isoxazol-5-yl)phenyl)-N-(2-(phenylsulfonyl)ethyl)acetamide (Cpd. 1045)

2,6-Dichloro-N-hydroxybenzimidoyl chloride (90 mg, 0.40 mmol) and 2,2-dichloro-N43-ethynylphenyl)-N-(2-(phenylsulfonyl)ethyl)acetamide (120 mg, 0.21 mmol) were dissolved in anhydrous tetrahydrofuran (20 mL) and triethylamine (0.75 mL, 0.40 mmol). The mixture was stirred at room temperature for 15 min, then heated at reflux for 4h. The solvent was removed under reduced pressure. The residue was dissolved in ethyl acetate and washed successively with water and brine. The ethyl acetate solution was dried over anhydrous sodium sulfate, filtered and concentrated under reduced pressure. The resulting solid was purified by flash column chromatography on silica gel, eluting with 30:70 ethyl acetate:hexanes to give 2,2-dichloro-N-(3-(3-(2,6-dichlorophenyl)isoxazol-5-yl)phenyl,N-(2-(phenylsulfonyl)ethyl)acetamide (Cpd. 1045) as a white solid. ¹H NMR (CDCl₃): 7.89 (m, 3H), 7.87 (br s, 1H), 7.53-7.40 (m, 4H), 7.44-7.33 (m, 4H), 6.71 (s, 1H), 5.82 (s, 1H), 4.11 (t, 2H), 3.47 ppm (t, 2H). MW=584 confirmed by LC-MS, tᵣ=12.13 min (Method A) MH⁺=582-586.

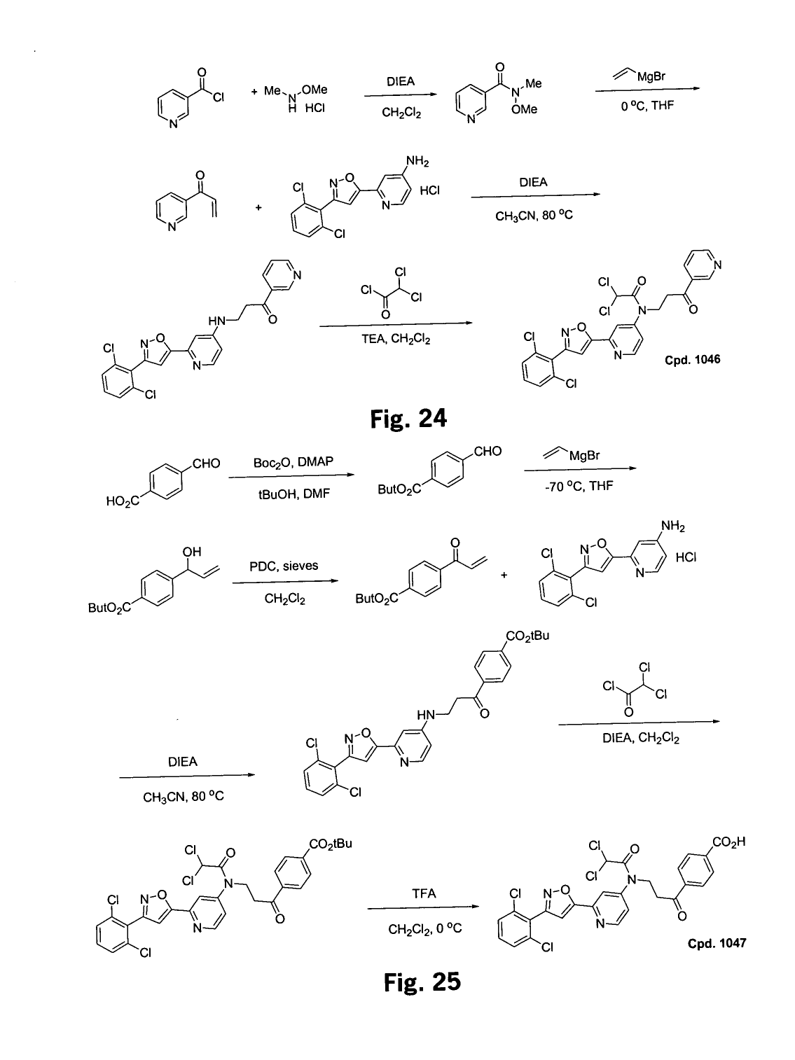

### Synthesis of 2.2-Dichloro-N-(2-(3-(2,6-dichlorophenyl)isoxazol-5-yl)pyridin-4-yl)-N-(3-oxo-3-fpyridin-3-yl)propyl)acetamide (See FIG. 24)(Cpd. 1046)

### Synthesis of N-Methoxy-N-methylnicotinamide

Diisopropylethylamine (33 mL, 190 mmol) was added, dropwise, to a solution of nicotinoyl chloride (8.9 g, 49.9 mmol) and N,O-dimethylhydroxylamine hydrochloride (6.6 g, 67.4 mmol) in anhydrous methylene chloride (120 mL) at 0° C. under nitrogen. The resulting mixture slowly warmed to room temperature overnight. The reaction was then diluted with methylene chloride and washed successively with water and brine. The aqueous layer was diluted with aqueous sodium bicarbonate solution and then extracted with methylene chloride. The combined organic layers were dried over anhydrous sodium sulfate, filtered and concentrated under reduced pressure. The resulting residue was purified by flash column chromatography on silica gel, eluting with 1:99 methanol:methylene chloride to provide N-methoxy-N-methylnicotinamide (6.8 g) as a yellow oil. ¹H NMR (CDCl₃): 8.93 (br s, 1H), 8.66 (m, 1H), 8.00 (m, 1H), 7.35 (m, 1H), 3.54 (s, 3H), 3.38 ppm (s, 3H).

### Synthesis of 1-(Pyridin-3-yl)prop-2-en-1-one

Vinylmagnesium bromide (IM soln in THF, 24 mL, 24 mmol) was added to a solution of N-methoxyN-methylnicotinamide (3.3 g, 19.8 mmol) in anhydrous tetrahydrofuran (40 mL) under nitrogen at 0° C., dropwise and then stirred for an hour at 0° C. The reaction warmed to room temperature and then stirred for 80 min. Methanol (10 mL) and acetic anhydride (10 mL) were added to the reaction mixture. The mixture was allowed to stir for 10 min, and then concentrated to a volume of 25 mL. The reaction mixture was washed with water and the aqueous layer was extracted with ethyl acetate. The combined organic layers were washed successively with aqueous sodium bicarbonate and brine, dried over anhydrous sodium sulfate, filtered and concentrated under reduced pressure at less than 40° C. The resulting residue was purified by flash column chromatography on silica gel, eluting with 98:2 methylene chloride:methanol to provide 14pyridin-3-yl)prop-2-en-1-one (1.1 g) as a yellow oil.

### Synthesis of 3-(2-(3-(2,6-Dichlorophenyl)isoxazol-5-yl)pyridin-4-ylamino)-1-(pyridin-3-yl)propan-1-one

3-(2,6-Dichlorophenyl)-5-(5-amino-2-pyridyl)isoxazole (503 mg, 1.47 mmol), 14pyridin-3-yl)prop-2-en-1-one (550 mg, 4.1 mmol) and triethylamine (0.62 mL, 4.4 mmol) were dissolved in anhydrous acetonitrile (20 mL). The mixture was heated at 70° C. for 27 h. By TLC the reaction was ˜50% complete but had not progressed during the last 5 h. The reaction mixture was concentrated under reduced pressure. The resulting residue was dissolved in methylene chloride and washed with brine. The organic layer was dried over anhydrous sodium sulfate, filtered and concentrated under reduced pressure. The resulting residue was purified by chromatotron, eluting with 4:96 methanol:methylene chloride twice, to provide 3-(2-(3-(2,6-dichlorophenyl)isoxazol-5-yl)pyridin4)ylamino)-1pyridin-3-yl)propan-1-one (270 mg) as a yellow oil. ¹H NMR (CDCl₃): 9.10 (m, 1H), 8.73 (m, 1H), 8.18 (m, 2H), 7.40-7.27 (m, 4H), 6.25 (s, 1H), 6.47 (m, 1H), 5.42 (m, 1H), 3.70 (m, 2H), 3.30 ppm (m, 2H). MW=439 confirmed by LC-MS, tᵣ=9.05 min (Method Y) MH⁺=437-441.

### Synthesis of 2,2-Dichloro-N-(2-(3-(2,6-dichlorophenyl)isoxazol-5-yl)pyridin-4-yl)-N-(3-oxo-3-(pyridin-3-yl)propyl)acetamide (Cpd. 1046)

3-(2-(3-(2,6-Dichlorophenyl)isoxazol-5-yl)pyridin-4-ylamino)-1-(pyridin-3-yl)propan-1-one (600 mg, 1.37 mmol) was dissolved in methylene chloride (10 mL) with triethylamine (381 μL, 2.73 mmol). The solution was cooled on an ice-water bath and then a solution of dichloroacetyl chloride (265 μL, 2.73 mmol) in methylene chloride (1 mL) was added dropwise. The reaction mixture was allowed to stir overnight while warming to room temperature. The solution was washed successively with water and saturated sodium bicarbonate solution, then dried over anhydrous sodium sulfate and concentrated under reduced pressure. The resulting residue was purified by flash column chromatography on silica gel, eluting with 2:98 methanol:methylene chloride to provide 2,2-dichloro-N-(2-(3-(2,6-dichlorophenyl)isoxazol-5-yl)pyridin4-yl)-N-(3-oxo-3-(pyridin-3-yl)propyl)acetamide (Cpd. 1046, lot 2, 220 mg) as a white solid. ¹H NMR (CDCl₃): 9.15 (s, 1H), 8.80 (m, 1H), 8.70 (s, 1H), 8.20 (m, 1H), 8.00 (m, 1H), 7.40-7.20 (m, 3H), 6.00 (s, 1H), 4.30 (m, 2H), 3.40 ppm (m, 2H). MW=550 confirmed by LC-MS, tᵣ=12.73 min (Method Y) MH⁺=548-552.

### Synthesis of 4-(3-(2.2-Dichloro-N-(2-(3-(2.6.dichlorophenyl)isoxazol-5-yl)pyridin-4-yl)acetamido)Dropanoyl)benzoic Acid (See FIG. 25) (Cpd. 1047)

### Synthesis of tert-Butyl 4-Formylbenzoate

4-Carboxybenzaldehyde (2.0 g, 13.3 mmol), 4-dimethylaminopyridine (0.41 g, 3.33 mmol), di-t-butyl dicarbonate (3.5 g, 16.0 mmol) and t-butanol (15 mL) were combined in dimethylformamide (30 mL) and heated at 50° C. overnight. The mixture was cooled to room temperature, poured into water and extracted with ether. The combined organic layers were washed successively with aqueous sodium bicarbonate and brine, dried over anhydrous sodium sulfate, filtered and concentrated under reduced pressure. The resulting residue was purified by flash column chromatography, on silica gel, eluting with 4:1 hexanes:ethyl acetate to provide tert-butyl 4-formylbenzoate (1.42 g) as a colorless oil. ¹H NMR (CDCl₃): 10.05 (s, 1H), 8.10 (d, 2H), 7.89 (d, 2H), 1.60 ppm (s, 9H).

### Synthesis of tert-Butyl 4-(1-Hydroxyallyl)benzoate

Vinylmagnesium bromide (1M soln in THF, 58 mL, 58 mmol) was added to a solution of the above aldehyde (9.88 g, 48 mmol) in anhydrous tetrahydrofuran (80 mL) under nitrogen at −70° C., dropwise and then stirred for 30 min at −70° C. The reaction was quenched cold with saturated aqueous ammonium chloride. The mixture was extracted with ethyl acetate and the combined organic layers were washed brine, dried over anhydrous sodium sulfate, filtered and concentrated under reduced pressure at less than 40° C. The resulting residue was purified by flash column chromatography on silica gel, eluting with 9:1 hexanes:ethyl acetate to provide tert-butyl 441-hydroxyallyl)benzoate (7.03 g) as a pale yellow oil. ¹H NMR (CDCl₃): 7.94 (d, 2H), 7.39 (d, 2H), 6.00 (m, 1H), 5.36 (m, 1H), 5.22-5.18 (m, 2H), 2.22 (br s, 1H), 1.59 ppm (s, 9H).

### Synthesis of tert-Butyl 4-Acryloylbenzoate

tert-Butyl 4-(1-hydroxyallyl)benzoate (7.03 g, 30.0 mmol), molecular sieves (4 Å, powdered, 5.6 g) and pyridinium dichromate (13.6 g, 36 mmol) were combined in methylene chloride (400 mL) and allowed to stir at room temperature for 4 h. The reaction mixture was filtered through a pad of Celite and the filtrated concentrated under reduced pressure. The resulting residue was purified by flash column chromatography, on silica gel, eluting with 95:5 hexanes:ethyl acetate to provide tert-butyl 4-acryloylbenzoate (1.2 g) as a yellow oil. ¹H NMR (CDCl₃): 8.07 (d, 2H), 7.93 (d, 2H), 7.12 (m, 1H), 6.43 (m, 1H), 5.97 (m, 1H), 1.60 ppm (s, 9H).

### Synthesis of tert-Butyl 4-(3-(2-(3-(2,6-Dichlorophenyl)isoxazol-5-yl)pyridin-4-ylamino)propanoyl)benzoate

3-(2,6-Dichlorophenyl)-5-(5-amino-2-pyridyl)isoxazole (300 mg, 0.88 mmol), tertbutyl 4-acryloylbenzoate (225 mg, 0.97 mmol) and triethylamine (0.27 mL, 1.94 mmol) were dissolved in anhydrous acetonitrile (4 mL) in a sealed vial. The mixture was heated at 80° C. overnight. The reaction mixture was concentrated under reduced pressure. The resulting residue was dissolved in methylene chloride and washed with brine. The organic layer was dried over anhydrous sodium sulfate, filtered and concentrated under reduced pressure. The crude product was purified first on a plug of silica gel, eluting with 1:9 methanol:methylene chloride and then purified by chromatotron, eluting with 1:1 hexanes:ethyl acetate, to provide tertbutyl 4-(3-(2-(3-(2,6-dichlorophenyl)isoxazol-5-yl)pyridin4-ylamino)propanoyl)benzoate (78 mg) as a white solid. MW=538 confirmed by LC-MS, tᵣ=12.61 min (Method Y) MH⁺=536-540.

### Synthesis of tert-Butyl 4-(3-(2,2-Dichloro-N-(2-(3-(2,6-dichlorophenyl)isoxazol-5-yl)pyridin-4-yl)acetamido)propanoyl)benzoate

tert-Butyl 4-(3-(2-(3-(2,6-dichlorophenyl)isoxazol-5-yl)pyridin-4-ylamino)propanoyl)benzoate (58 mg, 0.13 mmol) was dissolved in methylene chloride (5 mL) with diisopropylethylamine (23 μL, 0.16 mmol). The solution was cooled on an ice-water bath and then a solution of dichloroacetyl chloride (13 μL, 0.16 mmol) in methylene chloride (1 mL) was added dropwise. The reaction mixture was allowed to stir overnight while warming to room temperature. The solution was washed successively with water and saturated sodium bicarbonate solution, then dried over anhydrous sodium sulfate and concentrated under reduced pressure. The resulting residue was purified by flash column chromatography on silica gel, eluting with 1:1 hexanes:ethyl acetate to provide tert-butyl 4-(3-(2,2-dichloro-N-(2-(3-(2,6-dichlorophenyl)isoxazol-5-yl)pyridin4-yl)acetamido)propanoyl)benzoate (40 mg) as a white solid. ¹H NMR (CDCl₃): 8.80 (m, 1H), 8.10 (m, 4H), 7.40 (m, 3H), 7.10 (s, 1H), 6.00 (s, 1H), 4.20 (m, 2H), 3.50 (m, 2H), 1.60 ppm (s, 9H). MW=649 confirmed by LC-MS, tᵣ=18.03 min (Method Y) MH⁺=647-651.

### Synthesis of 4-(3-(2,2-Dichloro-N-(2-(3-(2,6-dichlorophenyl)isoxazol-5-yl)pyridin-4-yl)acetamido)propanoyl)benzoic Acid (Cpd. 1047)

Trifluoroacetic acid (2 mL) was added to a solution of tert-butyl 44342,2-dichloro-N-(2-(3-(2,6-dichlorophenyl)isoxazol-5-yl)pyridin4-yl)acetamido)propanoyl)benzoate (35 mg, 0.064 mmol) in methylene chloride (2 mL) at 0° C. The resulting mixture was allowed to stir at 0° C. for 5 hours, then concentrated under reduced pressure. The residue was dissolved in methylene chloride (2 mL) and concentrated under reduced pressure, twice more. And then lyophilized to produce 4-(3-(2,2-dichloro-N-(2-(3-(2,6-dichlorophenyl)isoxazol-5-yl)pyridin4-yl)acetamido)propanoyl)benzoic acid (Cpd. 1047, 27 mg) as a white solid. ¹H NMR (CDCl₃): 8.91 (m, 1H), 8.20 (m, 2H), 8.00 (m, 2H), 7.40 (m, 3H), 7.10 (s, 1H), 6.00 (s, 1H), 4.30 (m, 2H), 3.50 ppm (m, 2H). MW=593 confirmed by LC-MS, tᵣ=14.56 min (Method Y) MH⁺=591-595.

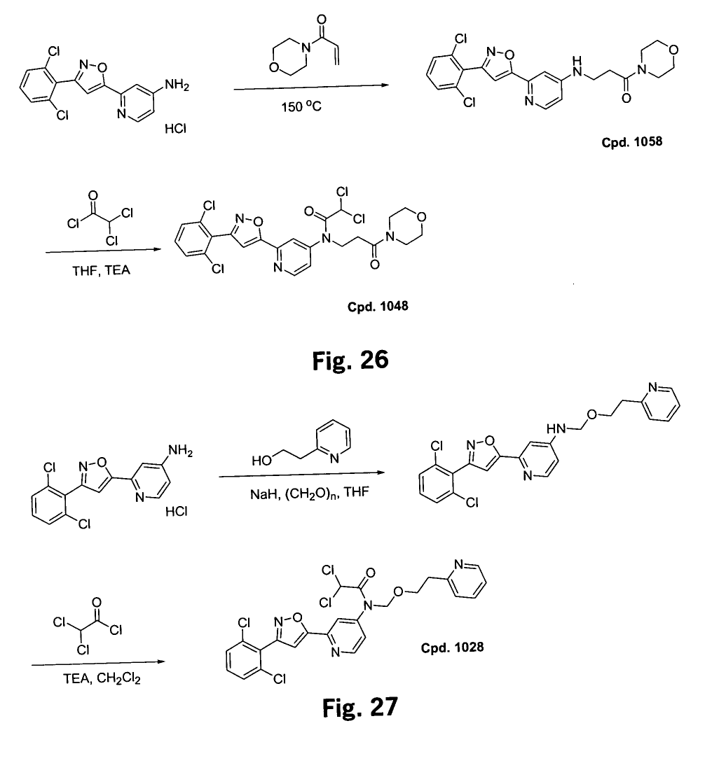

### Synthesis of 2,2-Dichloro-N-(2-(3-(2.6-dichlorophenyl)isoxazol-5-yl)pyridin-4-yl)-N-(3-morpholino-3-oxopropyl)acetamide(See FIG. 26) (Cpd. 1048)

### Synthesis of 3-(2-(3-(2,6-Dichlorophenyl)isoxazol-5-yl)pyridin-4-ylamino)-1-morpholinopropan-1-one

A mixture of 2-(3-(2,6-dichlorophenyl)isoxazol-5-yl)pyridin-4-amine hydrochloride (50 mg, 0.15 mmol) and 4-acryloylmorpholine (22 μL, 0.18 mmol) was heated at 150° C. for 17 h whereupon the LC-MS confirmed the starting material was consumed. The resulting residue was purified by flash column chromatography on silica gel, eluting with 98:2 methylene chloride:methanol to give 3-(2-(3-(2,6-dichlorophenyl)isoxazol-5-yl)pyridin-4-ylamino)-1-morpholinopropan-1-one (29 mg) as a pale yellow oil. ¹H NMR (CDCl₃): 8.28 (d, 1H), 7.47 (d, 1H), 7.44 (d, 1H), 7.36 (dd, 1H), 7.20 (d, 1H), 7.04 (s, 1H), 6.52 (dd, 1H), 5.37 (m, 1H), 3.74-3.64 (m, 8H), 3.51-3.46 (m, 2H), 2.67 ppm (t, 1H). MW=447 confirmed by LC-MS, tᵣ=8.89 min (Method Y) MH⁺=445-449.

### Synthesis of 2,2-Dichloro-N-(2-(3-(2,6-dichlorophenyl)isoxazol-5-yl)pyridin-4-yl)-N-(3-morpholino-3-oxopropyl)acetamide (Cpd. 1048)

3-(2-(3-(2,6-Dichlorophenyl)isoxazol-5-yl)pyridin4ylamino)-1-morpholinopropan-1-one (25 mg, 0.05 mmol) was dissolved in anhydrous tetrahydrofuran (1 mL) with triethylamine (20 μL, 0.14 mmol). The mixture was cooled in an ice-bath under nitrogen, then a solution of dichloroacetyl chloride (8.1 μL, 0.08 mmol) in anhydrous tetrahydrofuran (0.5 mL) was added dropwise. After the addition was completed the ice-bath was removed and the mixture was stirred at room temperature for 2 h. The reaction mixture was diluted with methylene chloride and then washed successively with water, 10% hydrochloric acid and saturated sodium bicarbonate solution. The organic solution was dried over anhydrous sodium sulfate, filtered and concentrated under reduced pressure. The resulting residue was purified by flash column chromatography on silica gel, eluting with 98:2 methylene chloride:methanol to give 2,2-dichloro-N(2-(3-(2,6-dichlorophenyl)isoxazol-5-yl)pyridin4yl)-N-(3-morpholino-3-oxopropyl)acetamide (Cpd. 1048, 17 mg) as a white solid. ¹H NMR (CDCl₃): 8.85 (d, 1H), 7.94 (d, 1H), 7.50 (d, 1H), 7.47 (d, 1H), 7.41 (d, 1H), 7.39 (m, 1H), 7.14 (s, 1H), 6.02 (br s, 1H), 3.75-3.68 (m, 6H), 3.63-3.60 (m, 2H), 3.56-3.53 (m, 2H), 2.81 ppm (t, 2H). MW=558 confirmed by LC-MS, tᵣ=13.41 min (Method Y) MH⁺=556-560.

Cpd. 1045: 2,2-Dichloro-N3-(3-(2,6-dichlorophenyl)isoxazol-5-yl)phenyl)-N-(2-(phenylsulfonyl)ethyl)acetamide; MW=584 confirmed by LC-MS, tᵣ=12.13 min (Method A) MH⁺=582-586.

Cpd. 1049: Ethyl 2-(4-(3-(2,2-Dichloro-N-(3-(3-(2,6-dichlorophenyl)isoxazol-5-yl)phenyl)acetamido)propanoyl)phenyl)acetate; MW=633 confirmed by LC-MS, tᵣ=12.84 min (Method A) MH⁺=631-635.

Cpd. 1046: 2,2-Dichloro-N-(2-(3-(2,6-dichlorophenyl)isoxazol-5-yl)pyridin-4-yl)-N-(3-oxo-3-(pyridin-3-yl)propyl)acetamide; MW=550 confirmed by LC-MS, tᵣ=14.04 min (Method Y) MH⁺=548-552.

Cpd. 1050: N-(4-Amino-3-oxo-5-phenylpentyl)-2,2-dichloro-N-(2-(3-(2,6-dichlorophenyl)isoxazol-5-yl)pyridin-4-yl)acetamide; MW=592 confirmed by LC-MS, tᵣ=11.41 min (Method A) MH⁺=590-594.

Cpd. 1051: 2,2-Dichloro-N-(2-(3-(2,6-dichlorophenyl)isoxazol-5-yl)pyridin-4-yl)-N-(3-oxo-3-(piperidin4yl)propyl)acetamide; MW=556 confirmed by LC-MS, tᵣ=9.99 min (Method Y) MH⁺=554-558.

Cpd. 1052: 2,2-Dichloro-N-(2-(3-(2,6-dichlorophenyl)isoxazol-5-yl)pyridin4-yl)-N-(3-oxo-3-(pyrrolidin-2-yl)propyl)acetamide; MW=542 confirmed by LC-MS, tᵣ=10.47 min (Method Y) MH⁺=540-544.

Cpd. 1053: tert-Butyl 3-(3-(2,2-Dichloro)-N-(2-(3-(2,6-dichlorophenyl)isoxazol-5-yl)pyridin4yl)acetamido)propanoyl)piperidine-1-carboxylate; MW=655 confirmed by LC-MS, tᵣ=17.05 min (Method Y) MH⁺=653-657.

Cpd. 1054: 2,2-Dichloro-N-(2-(3-(2,6-dichlorophenyl)isoxazol-5-yl)pyridin-4-yl),N3-oxo-3-(piperidin-3-yl)propyl)acetamide; MW=555 confirmed b y LC-MS, tᵣ=10.74 min (Method Y) MH⁺=553-557.

Cpd. 1055: tert-Butyl 4-(4-(3-(2,2-Dichloro-N-(2-(3-(2,6-dichlorophenyl)isoxazol-5-yl)pyridin-4-yl)acetamido)propanoyl)phenyl)piperazine-1-carboxylate; MW=733 confirmed by LC-MS, tᵣ=17.53 min (Method Y) MH⁺=731-735.

Cpd. 1056: tert-Butyl 4-(3-(2,2-Dichloro-N-(2-(3-(2,6-dichlorophenyl)isoxazol-5-yl)pyridin4yl)acetamido)propanoyl)benzoate; MW=649 confirmed by LC-MS, tᵣ=18.03 min (Method Y) MH⁺=647-651.

Cpd. 1047: 4-(3-(2,2-Dichloro-N-(2-(3-(2,6-dichlorophenyl)isoxazol-5-yl)pyridin4-yl)acetamido)propanoyl)benzoic acid; MW=593 confirmed by LC-MS, tᵣ=14.56 min (Method Y) MH⁺=591-595.

Cpd. 1048: 2,2-Dichloro-N-(2-(3-(2,6-dichlorophenyl)isoxazol-5-yl)pyridin4-yl)-N-(3-morpholino-3-oxopropyl)acetamide; MW=558 confirmed by LC-MS, tᵣ=13.41 min (Method Y) MH⁺=556-560.

### Synthesis of 4-((2,2-Dichloro-N-(2-(3-(2,6-dichlorophenyl)isoxazol-5-yl)pyridin-4-yl)acetamido)methyl)-2,6-dimethylphenyl Propylcarbamate (Cpd. 1057)

### Synthesis of 4-Formyl-2,6-dimethylphenyl Propylcarbamate

1-Isocyanatopropane (0.727 ml, 7.66 mmol) was added dropwise to a suspension of 4-hydroxy-3,5-dimethylbenzaldehyde (1 g, 6.66 mmol) and potassium carbonate (1.66 g, 12 mmol) at 0° C. in tetrahydrofuran (6 mL). The suspension was stirred at 0° C. for 30 min and then allowed to warm to room temperature and stirred for an additional 30 min. The suspension was concentrated under reduced pressure and the resulting residue was dissolved in ethyl acetate. The solution was washed with water, then dried over anhydrous sodium sulfate, filtered and concentrated under reduced pressure. The crude product was purified by flash column chromatography, on silica gel, eluting with 8:2 hexanes:ethyl acetate to yield 4-formyl-2,6-dimethylphenyl propylcarbamate.

### Synthesis of 4-((2-(3-(2,6-Dichlorophenyl)isoxazol-5-yl)pyridin-4-ylamino)methyl)-2,6-dimethylphenyl Propylcarbamate

2-(3-(2,6-Dichlorophenyl)isoxazol-5-yl)pyridin4-amine hydrochloride (200 mg, 0.59 mmol) and 4-formyl-2,6-dimethylphenyl propylcarbamate (151 mg, 0.644 mmol) were combined in 1,2-dichloroethane (5 mL) and treated with sodium triacetoxyborohydride (248 mg, 1.17 mmol) and acetic acid (37 μL, 0.644 mmol). The mixture was stirred at room temperature overnight. The reaction was quenched by addition of saturated sodium bicarbonate solution. The crude product was extracted with ethyl acetate (2×50 mL). The organic extract was dried over anhydrous sodium sulfate, filtered and concentrated under reduced pressure. The resulting residue was purified by column chromatography, on silica gel, eluting with 99:1 dichloromethane:methanol to yield 4-((2-(3-(2,6-dichlorophenyl)isoxazol-5-yl)pyridin-4-ylamino)methyl)-2,6-dimethylphenyl propylcarbamate. ¹H NMR (CDCl₃): 8.2 (d, 1H), 7.4 (d, 1H), 7.2-7.3 (m, 2H), 7.0 (d, 2H), 6.4 (d, 1H), 5.1-5.2 (m, 2H), 4.3 (d, 2H), 3.2 (m, 2H), 2.2 (s, 6H), 1.6 (m, 2H), 1.0 ppm (m, 3H). MW=525 confirmed by LC-MS tᵣ=11.71 (Method Y1) MH⁺=523-527.

### Synthesis of 4-((2,2-Dichloro-N-(2-(3-(2,6-dichlorophenyl)isoxazol-5-yl)pyridin-4-yl)acetamido)methyl)-2,6-dimethylphenyl Propylcarbamate (Cpd. 1057)

4-((2-(3-(2,6-Dichlorophenyl)isoxazol-5-yl)pyridin-4-ylamino)methyl)-2,6-dimethylphenyl propylcarbamate (60 mg, 0.11 mmol) was dissolved in anhydrous dichloromethane with triethylamine (0.1 mL). The mixture was cooled in an ice-bath under nitrogen, then a solution of dichloroacetyl chloride (0.046 mL, 0.13 mmol) was added dropwise. After the addition was completed the ice-bath was removed and the mixture was stirred at room temperature overnight. The reaction mixture was diluted with dichloromethane and then washed successively with water, 10% hydrochloric acid and saturated sodium bicarbonate solution. The organic solution was dried over anhydrous sodium sulfate, filtered and concentrated under reduced pressure. The resulting residue was purified by flash column chromatography, on silica gel, eluting with 30:70 ethyl acetate:hexanes to give 4-((2,2-dichloro-N-(2-(3-(2,6-dichlorophenyl)isoxazol-5-yl)pyridin-4-yl)acetamido)methyl)-2,6-dimethylphenyl propylcarbamate (Cpd. 1057). ¹H NMR (CDCl₃): 8.7 (d, 1H), 7.8 (s, 1H), 7.4 (m, 1H), 7.3 (m, 1H), 7.1 (m, 2H), 6.9 (s, 2H), 6.0 (s, 1H), 5.1 (m, 1H), 5.0 (s, 2H), 3.2 (m, 2H), 2.1 (s, 6H), 2.6 (m, 2H), 1.0 ppm (m, 3H). MW=636 confirmed by LC-MS, tᵣ=16.41 min (Method Y1) MH⁺=634-638.

### Synthesis of 2,2-Dichloro-N¹-(3-(3-(2,6-dichlorophenyl)isoxazol-5-yl)phenyl)-N³-methylmalonamide (Cpd. 1010)

### Synthesis of Methyl 2,2,3-Trichloro-3-oxopropanoate

Methyl malonyl chloride (3.2 g, 23,5 mmol) was heated with thionyl chloride (8 mL) and aluminum chloride (50 mg) under reflux for 5 h. The crude product was then distilled at ˜20 mmHg to give methyl 2,2,3-trichloro-3-oxopropanoate (3.9 g) as a colorless liquid. This follows the procedure given by G. Castelfranchi and T. Perrofti, Annali di Chimica, 47, 1201-1224 (1957, Chemical Abstracts CA 52:40383). ¹H NMR (CDCl₃): 4.00 ppm (s, 3H).

### Synthesis of Methyl 2,2-Dichloro-3-(3-ethynylphenylamino)-3-oxopropanoate (FIG. 29)

3-Ethynylaniline (1.64 g, 14 mmol) was dissolved in dichloromethane (50 mL) with triethylamine (2.6 mL, 42 mmol). The resulting mixture was cooled in an ice-bath under nitrogen and then methyl 2,2,3-trichloro-3-oxopropanoate (3.2 g, 15.6 mmol) was added dropwise. The reaction mixture was allowed to warm to room temperature while stirring overnight. The reaction mixture was washed successively with water, 10% hydrochloric acid and saturated sodium bicarbonate solution. The organic solution was dried over anhydrous sodium sulfate, filtered and concentrated under reduced pressure to give methyl 2,2-dichloro-3-(3-ethynylphenylamino)-3-oxopropanoate (1.34 g) as an amber syrup. The product was carried forward without further purification. ¹H NMR (CDCl₃): 8.40 (br s, 1H), 7.69 (m, 1H), 7.58 (m, 1H), 7.34 (m, 2H), 3.96 (s, 3H), 3.10 ppm (s, 1H).

### Synthesis of Methyl 2,2-Dichloro-3-(3-(3-(2,6-dichlorophenyl)isoxazol-5-yl)phenylamino)-3-oxopropanoate

Methyl 2,2-dichloro-3-(3-ethynylphenylamino)-3-oxopropanoate (1.34 g, 4.68 mmol) was dissolved in anhydrous tetrahydrofuran (20 mL) and treated with triethylamine (0.86 mL, 6.1 mmol) and then 2,6-dichloro-N-hydroxybenzimidoyl chloride (1.05 g, 4.71 mmol). After stirring 15 min at room temperature the mixture was heated at reflux for 4 h. The mixture was diluted with ethyl acetate, washed with brine, dried over anhydrous sodium sulfate, filtered and concentrated under reduced pressure. The resulting residue was purified by flash column chromatography, on silica gel, eluting with 2:8 ethyl acetate:hexanes to give methyl 2,2-dichloro-3-(3-(3-(2,6-dichlorophenyl)isoxazol-5-yl)phenylamino)-3-oxopropanoate (1.27 g) as a white solid. ¹H NMR (CDCl₃): 8.54 (br s, 1H), 8.09 (m, 1H), 7.72 (m, 1H), 7.66 (m, 1H), 7.52 (m, 1H), 7.44 (m, 2H), 7.35 (m, 1H), 6.67 (s, 1H), 3.98 ppm (s, 3H). MW=474 confirmed by LC-MS, tᵣ=15.97 min (Method Y1) MH⁺=472-476.

### Synthesis of 2,2-Dichloro-N¹-(3-(3-(2,6-dichlorophenyl)isoxazol-5-yl)phenyl)-N³-methylmalonamide (Cpd. 1010)

Methyl 2,2-dichloro-3-(3-(3-(2,6-dichlorophenyl)isoxazol-5-yl)phenylamino)-3-oxopropanoate (112 mg, 0.24 mmol) was dissolved in anhydrous tetrahydrofuran (2 mL). The mixture was treated with powered molecular sieves (4 Å, 200 mg) and bubbled with argon. Then the N-heterocyclic carbene catalyst (S. Nolan, J. Org. Chem, 68, 2812, 2003), 1,3-bis(1-adamantyl)imidazol-2-ylidene (11 mg) was added, followed by a solution of methylamine (1 mL, 2 M soln in THF). The mixture was shaken for 3 h at room temperature, then diluted with ethyl acetate, washed with water, dried over anhydrous sodium sulfate, filtered and concentrated under reduced pressure. The resulting residue was purified by flash column chromatography, on silica gel, eluting with 1:1 ethyl acetate:hexanes to give 2,2-dichloro-N¹-(3-(3-(2,6-dichlorophenyl)isoxazol-5-yl)phenyl)-N³-methylmalonamide (31 mg) as a white solid. ¹H NMR (CDCl₃): 9.36 (br s, 1H), 8.10 (s, 1H), 7.69 (m, 1H), 7.63 (m, 1H), 7.49-7.41 (m, 3H), 7.32 (m, 1H), 6.60 (m, 1H), 2.99 ppm (m, 3H). MW=474 confirmed by LC-MS, tᵣ=14.45 min (Method Y1) MH⁺=472-476.

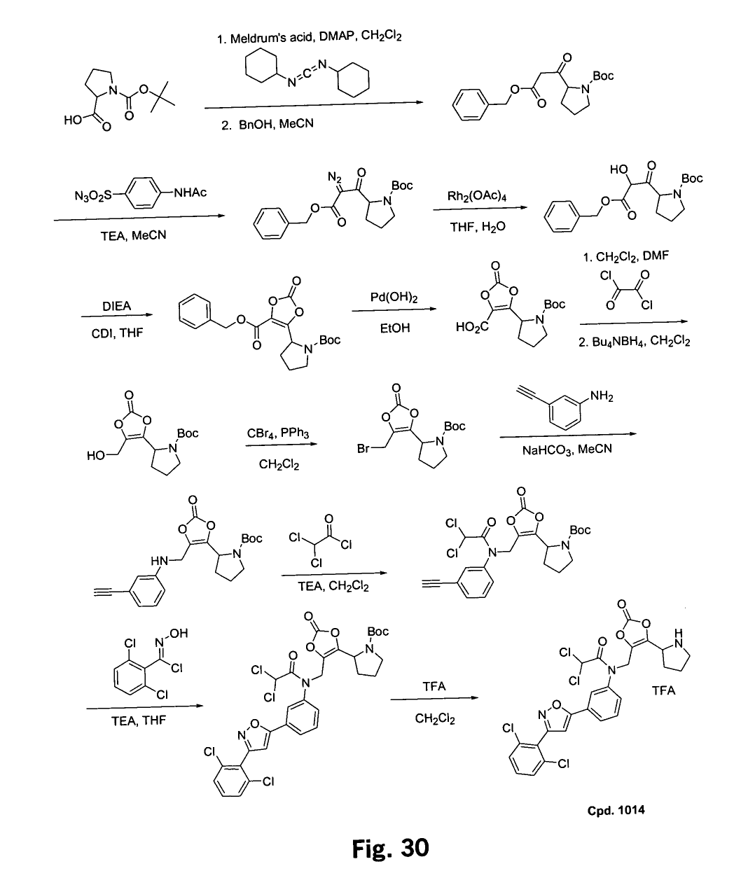

### Synthesis of 2,2-Dichloro-N-(3-(3-(2,6-dichlorophenyl)isoxazol-5-yl)phenyl)-N-((2-oxo-5-(pyrrolidin-2-yl)-1,3-dioxol-4-yl)methyl)acetamide Trifluoroacetate Salt (See FIG. 30) (Cpd. 1014)

### Synthesis of tert-Butyl 2-(3-(Benzyloxy)-3-oxopropanoyl)pyrrolidine1-Ccarboxylate (FIG. 30)

L-Boc-proline (16.1 g, 75 mmol), Meldrum's acid (10.8 g, 75 mmol) and 4-dimethylaminopyridine (18.3 g, 150 mmol) were dissolved in dichloromethane (250 mL). Then dicyclohexylcarbodiimide (15.5 g, 75 mmol) was added in several portions. The mixture stirred at room temperature for 2.5 d, followed by filtration through a pad of Celite. The filtrate was washed successively with water, 10% hydrochloric acid and saturated sodium bicarbonate solution. The organic solution was dried over anhydrous sodium sulfate, filtered and concentrated under reduced pressure to give a yellow foam. The crude product was dissolved in acetonitrile (100 mL) with benzyl alchohol (8.11 g, 75 mmol) and heated at reflux for 6 h. The reaction mixture was concentrated under reduced pressure to give tert-butyl 2-(3-benzyloxy)-3-oxopropanoyl)pyrrolidine-1-carboxylate. The product was carried forward without further purification. ¹H NMR (CDCl₃): 7.32 (s, 5H), 5.16 (s, 2H), 4.30 (m, 1H), 3.62-3.30 (m, 4H), 2.20-1.78 (m, 4H), 1.38 ppm (s, 9H).

### Synthesis of tert-Butyl 2-(3-(Benzyloxy)-2-diazo-3-oxopropanoyl)pyrrolidine-1-carboxylate

tert-Butyl 2-(3-(benzyloxy)-3-oxopropanoyl)pyrrolidine-1-carboxylate was dissolved in acetonitrile (500 mL) and treated with 4-acetamidobenzenesulfonyl azide (18 g, 75 mmol). The solution was cooled in an ice-bath and then triethylamine (32 mL, 225 mmol) was added dropwise. After 1 h, the ice-bath was removed and the reaction was allowed to slowly warm to room temperature for 4 h. The solvent was concentrated under reduced pressure and the crude product was triturated with ether:hexanes (2:1) and filtered. The filtrate was concentrated and the resulting residue was purified by flash column chromatography, on silica gel, eluting with 4:6 ethyl acetate:hexanes to give tertbutyl 2-(3-(benzyloxy)-2-diazo-3-oxopropanoyl)pyrrolidine-1-carboxylate (22.7 g) as an oil. ¹H NMR (CDCl₃): 7.35 (m, 5H), 5.35-5.15 (m, 3H), 3.62-3.38 (m, 2H), 2.22 (m, 1H), 1.82 (m, 3H), 1.44 (s) and 1.38 ppm (s) (9H, NHBoc, 2 carbamate rotamers).

### Synthesis of tert-Butyl 2-(3-(Benzyloxy)-2-hydroxy-3-oxopropanoyl)pyrrolidine-1-carboxylate

tert-Butyl 2-(3-(benzyloxy)-2-diazo-3-oxopropanoyl)pyrrolidine-1-carboxylate (22.7 g) was dissolved in a mixture of tetrahydrofuran (400 mL) and water (200 mL) and treated with rhodium (II) acetate dimer (0.69 g). The mixture was heated at 100 OC for 5 h. The tetrahydrofuran was removed by concentration under reduced pressure and the aqueous solution was extracted (3×200 mL) with ethyl acetate. The combined organic layers were washed with brine, dried over anhydrous sodium sulfate, filtered and concentrated under reduced pressure to give tertbutyl 2-(3-(benzyloxy)-2-hydroxy-3-oxopropanoyl)pyrrolidine-1-carboxylate (17.43 g) as a brown oil. The product was carried forward without further purification.

### Synthesis of tert-Butyl 2-(5-(Benzyloxycarbonyl)-2-oxo-1,3-dioxol-4-yl)pyrrolidine-1-carboxylate

tert-Butyl 2434benzyloxy)-2-hydroxy-3-oxopropanoyl)pyrrolidine-1-carboxylate (17.43 g, 48.0 mmol) was dissolved in anhydrous tetrahydrofuran (400 mL) and treated with 1,1-carbodiimidazole (15.57 g, 96.0 mmol) and diisopropylethylamine (2 mL). The mixture was stirred for 2.5 days at room temperature. The mixture was concentrated under reduced pressure and partitioned between ethyl acetate and 1M aqueous potassium bisulfate. The organic layer was washed with water and brine and then dried over anhydrous sodium sulfate, filtered and concentrated under reduced pressure. The resulting residue was purified by flash column chromatography, on silica gel, to give tert-butyl 2-(5-(benzyloxycarbonyl)-2-oxo-1,3-dioxol-4-yl)pyrrolidine-1-carboxylate (4.14 g). ¹H NMR (CDCl₃): 7.38 (s, 5H), 5.32 (s, 2H), 5.40-5.20 (m, 1H), 3.60-3.40 (m, 2H), 2.30 (m, 1H), 2.10-1.85 (m, 3H), 1.45 (s) and 1.30 ppm (s) (9H, NHBoc, 2 carbamate rotamers).

### Synthesis of 5-(1-(tert-Butoxycarbonyl)pyrrolidin-2-yl)-2-oxo-1,3-dioxole-4-carboxylic Acid

tert-Butyl 2-(5-(benzyloxycarbonyl)-2-oxo-1,3-dioxol4-yl)pyrrolidine-1-carboxylate (4.14 g) was dissolved in ethanol (100 mL) and treated with 20% palladium hydroxide on carbon (530 mg) followed by shaking under 16 psi hydrogen for 1 h. The mixture was filtered through a pad of Celite and the filtrate was concentrated under reduced pressure to provide 5-(1-(tert-butoxycarbonyl)pyrrolidin-2-yl)-2-oxo-1,3-dioxole4-carboxylic acid (2.76 g) as an off white solid. ¹H NMR (CDCl₃): 7.87 (br s, 1H), 5.30 (m, 1H), 3.60-3.30 (m, 2H), 2.40-1.80 (m, 4H), 1.40 ppm (br s, 9H).

### Synthesis of tert-Butyl 2-(5-(Hydroxymethyl)-2-oxo-1,3-dioxol-4-yl)pyrrolidine-1-carboxylate

5-(1-(tert-Butoxycarbonyl)pyrrolidin-2-yl2-oxo-1,3-dioxole-4-carboxylic acid (2.76 g, 9.68 mmol) was dissolved in dichloromethane (50 mL) and treated with oxalyl chloride (1.31 mL, 14.5 mmol) followed by several drops of dimethylformamide. After 1 h, the mixture was concentrated under reduced pressure. The residue was dissolved in anhydrous dichloromethane (40 mL) and cooled to −70° C. under argon. Then a solution of tetrabutylammonium borohydride (2.85 g, 11.0 mmol) in anhydrous dichloromethane (20 mL) was added dropwise. After an hour at −70° C. the reaction was quenched with 1M aqueous potassium bisulfate (15 mL), followed by warming to room temperature. The reaction was concentrated under reduced pressure and the residue was partitioned between ethyl acetate and water. The aqueous layer was extracted twice with ethyl acetate and the combined organic layers were washed with brine, dried over anhydrous sodium sulfate, filtered and concentrated under reduced pressure. The crude product was purified by flash column chromatography, on silica gel, eluting with 1:1 ethyl acetate:hexanes to give tert-butyl 2-(5-(hydroxymethyl)-2-oxo-1,3-dioxol4yl)pyrrolidine-1-carboxylate (900 mg) as an oil. ¹H NMR (CDCl₃): 4.83 (m, 1H), 4.53 (m, 1H), 4.29 (m, 1H), 3.39 (m, 3H), 2.12 (m, 2H), 1.93 (m, 1H), 1.42 ppm (s, 9H).

### Synthesis of tert-Butyl 2-(5-(Bromomethyl)-2-oxo-1,3-dioxol-4-yl)pyrrolidine-1-carboxylate

tert-Butyl 2-(5-(hydroxymethyl)-2-oxo-1,3-dioxol-4-yl)pyrrolidine-1-carboxylate (900 mg, 3.3 mmol) was dissolved in anhydrous dichloromethane (20 mL) and treated with carbon tetrabromide (1.31 g, 3.96 mmol). After cooling in an ice-bath under nitrogen, triphenylphosphine (950 mg, 3.63 mmol) was added. After 30 min, the ice-bath was removed and the mixture was stirred for 90 min at room temperature. The mixture was concentrated under reduced pressure and the resulting residue was purified by flash column chromatography, on silica gel, eluting with 2:8 ethyl acetate:hexanes to give tert-butyl 2-(5-(bromomethyl)-2-oxo-1,3-dioxol-4-yl)pyrrolidine-1-carboxylate (770 mg) as a pale yellow oil. ¹H NMR (CDCl₃): 4.70 (m, 1H), 4.42 (m, 1H), 4.19 (m, 1H), 3.50 (m, 2H), 2.40-1.80 (m, 4H), 1.41 ppm (s, 9H).

### Synthesis of tert-Butyl 2-(5-((3-Ethynylphenylamino)methyl)-2-oxo-1,3-dioxol-4-yl)pyrrolidine-1-carboxylate

tert-Butyl 2-(5-(bromomethyl)-2-oxo-1,3-dioxol-4-yl)pyrrolidine-1-carboxylate (770 mg, 2.21 mmol) was dissolved in acetonitrile (10 mL) and treated with 3-ethynyl aniline (260 mg, 2.21 mmol) and sodium bicarbonate (250 mg). The mixture was heated at 80° C. for 3 h. The mixture was concentrated under reduced pressure and the residue was partitioned between ethyl acetate and brine. The organic layer was dried over anhydrous sodium sulfate, filtered and concentrated under reduced pressure to give tert-butyl 2-(5-((3-ethynylphenylamino)methyl)-2-oxo-1,3-dioxol-4-yl)pyrrolidine-1-carboxylate, which was carried forward without further purification. ¹H NMR (CDCl₃): 7.07 (m, 1H), 6.86 (m, 1H), 6.75 (m, 1H), 6.62 (m, 1H), 4.75 (m, 1H), 4.37 (m, 2H), 4.12 (m, 1H), 3.41 (m, 2H), 3.00 (m, 1H), 2.20-1.80 (m, 4H), 1.46 ppm (s, 9H). Synthesis of tert-Butyl 2-(5-((2,2-Dichloro-N-(3-ethynylphenyl)acetamido)methyl)-2-oxo-1,3-dioxol-4-yl)pyrrolidine-1-carboxylate

tert-Butyl 2-(5-((3-ethynylphenylamino)methyl)-2-oxo-1,3-dioxol-4-yl)pyrrolidine-1-carboxylate (85 mg, 2.2 mmol) was dissolved in anhydrous dichloromethane (40 mL) with triethylamine (0.41 mL, 4.0 mmol). The mixture was cooled in an ice-bath under nitrogen, then a solution of dichloroacetyl chloride (0.29 mL, 3.0 mmol) was added dropwise. After the addition was completed the ice-bath was removed and the mixture was stirred at room temperature overnight. The reaction mixture was washed successively with 0.5M aqueous potassium bisulfate, water and saturated sodium bicarbonate solution. The organic solution was dried over anhydrous sodium sulfate, filtered and concentrated under reduced pressure. The resulting residue was purified by flash column chromatography, on silica gel, to give tert-butyl 2-(5-((2,2-dichloro-N-(3-ethynylphenyl)acetamido)methyl)-2-oxo-1,3-dioxol-4-yl)pyrrolidine-1-carboxylate (282 mg) as a beige solid. ¹H NMR (CDCl₃): 7.55 (m, 1H), 7.48 (m, 2H), 7.33 (m, 1H), 5.79 (s, 1H), 4.93 (m, 1H), 4.75 (m, 1H), 4.55 (m, 1H), 3.38 (m, 2H), 3.18 (s, 1H), 2.20-1.80 (m, 4H), 1.41 ppm (s, 9H).

### Synthesis of tert-Butyl 2-(5-((2,2-Dichloro-N-(3-(3-(2,6-dichlorophenyl)isoxazol-5-yl)phenyl)acetamido)methyl)-2-oxo-1,3-dioxol-4-yl)pyrrolidine-1-carboxylate

tert-Butyl 2-(5-((2,2-dichloro-N-3-ethynylphenyl)acetamido)methyl 2-oxo-1,3-dioxol-4-yl)pyrrolidine-1-carboxylate (282 mg, 0.57 mmol) was dissolved in anhydrous tetrahydrofuran (10 mL) and treated with triethylamine (0.15 mL, 0.74 mmol) and then 2,6-dichloro-N-hydroxybenzimidoyl chloride (140 mg, 0.63 mmol). After stirring 15 min at room temperature the mixture was heated at reflux for 3 h. The mixture was diluted with ethyl acetate, washed with brine, dried over anhydrous sodium sulfate, filtered and concentrated under reduced pressure. The resulting residue was by flash column chromatography, on silica gel, eluting with 3:7 ethyl acetate:hexanes to give tert-butyl 2-(5-((2,2-dichloro-N-4-(4-(3-(2,6-dichlorophenyl)isoxazol-5-yl)phenyl)acetamido)methyl)-2-oxo-1,3-dioxol4-yl)pyrrolidine-1-carboxylate (310 mg) as a white solid. ¹H NMR (CDCl₃): 7.92 (m, 1H), 7.80 (m, 1H), 7.62 (m, 1H), 7.32 (m, 1H), 6.70 (s, 1H), 5.89 (s, 1H), 5.07 (m, 1H), 4.82 (m, 1H), 4.60 (m, 1H), 3.39 (m, 2H), 2.20-1.80 (m, 4H), 1.41 ppm (s, 9H). MW=705 confirmed by LC-MS, tᵣ=17.44 min (Method Y1) MH⁺Na=702-708.

### Synthesis of 2,2-Dichloro-N-(3-(3-(2,6-dichlorophenyl)isoxazol-5-yl)phenyl)-N-((2-oxo-5-(pyrrolidin-2-yl)-1,3-dioxol-4-yl)methyl)acetamide Trifluoroacetate Salt (Cpd. 1014)

tert-Butyl 2-(5-((2,2-dichloro-N-(3-(3-(2,6-dichlorophenyl)isoxazol-5-yl)phenyl)acetamido)methyl-2-oxo-1,3-dioxol4-yl)pyrrolidine-1-carboxylate (93 mg) was dissolved in anhydrous dichloromethane (3 mL) and cooled in an ice-bath under nitrogen. Then trifluoroacetic acid (2 mL) was added. After 90 min at 4° C. the mixture was concentrated under reduced pressure. The residue was dissolved and concentrated under reduced pressure again to give 2,2-dichloro-N-(3-(3-(2,6-dichlorophenyl)isoxazol-5-yl)phenyl)-N4(2-oxo-5-(pyrrolidin-2-yl)-1,3-dioxol-4-yl)methyl)acetamide trifluoroacetate salt (Cpd. 1014) as a solid. ¹H NMR (CDCl₃): 7.96 (m, 1H), 7.86 (m, 1H), 7.64 (m, 1H), 7.47 (m, 3H), 7.34 (m, 1H), 6.73 (s, 1H), 5.88 (s, 1H), 5.10 (m, 1H), 4.73 (m, 2H), 3.53 (m, 2H), 2.55-2.15 ppm (m, 4H). MW=584 confirmed by LC-MS, tᵣ=11.24 min (Method Y1) MH⁺=582-586.

### Synthesis of 6,6-Dichloro-4-(2-(3-(2,6-dichlorophenyl)isoxazol-5-yl)pyridin-4-yl)-1-(pyrrolidin-2-yl)hexane-1,5-dione (Cpd. 1024)

### Synthesis of tert-Butyl 2-Acryloylpyrrolidine-1-carboxylate

tert-Butyl 2-(methoxy(methyl)carbamoyl)pyrrolidine-1-carboxylate (6.0 g, 23.2 mmol) was dissolved in anhydrous tetrahydrofuran (100 mL) and cooled to −78° C. Upon cooling vinylmagnesium bromide (58.1 mL, 1.0 M soln in THF, 58.1 mmol) was added dropwise. The reaction was then heated at reflux for 2 h. The reaction mixture was cooled to 0° C., diluted with 1N hydrochloric acid (75 mL) and extracted with ethyl acetate. The combined organic layers were dried over anhydrous sodium sulfate, filtered and concentrated under reduced pressure. The resulting residue was purified by flash column chromatography, on silica gel, eluting with 8:2 hexanes:ethyl acetate, to provide tert-butyl 2-acryloylpyrrolidine-1-carboxylate (2.3 g) as an amber oil. ¹H NMR (CDCl₃): 6.01-5.89 (m, 2H), 5.50 (d, 2H), 5.22-5.17 (t, 2H), 3.94 (m, 1H), 3.59 (m, 1H), 3.17 (m, 1H), 1.80 (m, 1H), 1.44 ppm (s, 9H).

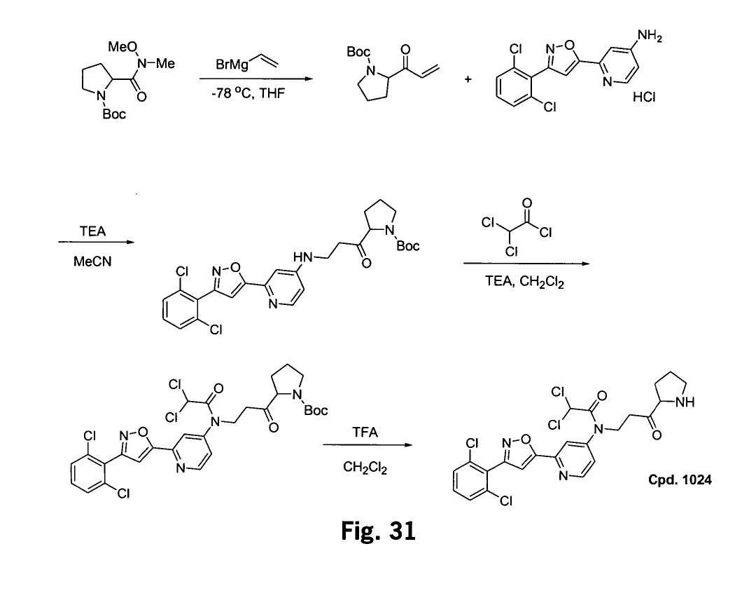

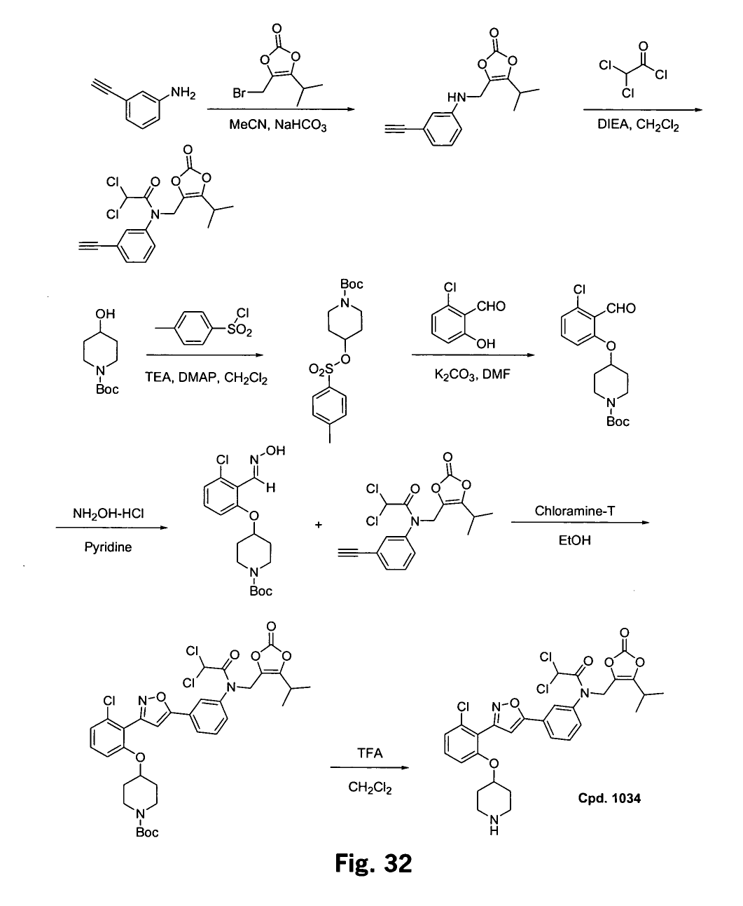

### Synthesis of tert-Butyl 2-(3-(2-(3-(2,6-Dichlorophenyl)isoxazol-5-yl)pyridin-4-ylamino)propanoyl)pyrrolidine-1-carboxylate (FIG. 31)

2-(3-(2,6-Dichlorophenyl)isoxazol-5-yl)pyridin-4-amine hydrochloride (400 mg, 1.2 mmol), tert-butyl 2-acryloylpyrrolidine-1-carboxylate (270 mg, 1.2 mmol) and triethylamine (0.42 mL, 3.0 mmol) were combined in acetonitrile (5 mL). The resulting solution was stirred in a sealed vial at 80° C. overnight. A second portion of tert-butyl 2-acryloylpyrrolidine-1-carboxylate (270 mg, 1.2 mmol) was added and heating was continued until desired product and starting material had a 1:1 ratio by LC-MS. The reaction mixture was cooled to room temperature and diluted with dichloromethane (10 mL). The organic mixture was washed successively with water and saturated sodium bicarbonate solution. The organic solution was dried over anhydrous sodium sulfate, filtered and concentrated under reduced pressure. The resulting residue was purified by flash column chromatography on silica gel, eluting with 1:1 ethyl acetate:hexanes to give tertbutyl 2-(3-(2-(3-(2,6-dichlorophenyl)isoxazol-5-yl)pyridin-4-ylamino)propanoyl)pyrrolidine-1-carboxylate (125 mg) as a pale yellow oil. ¹H NMR (CDCl₃): 8.22 (m, 1H), 7.44 (d, 2H), 7.38 (m, 1H), 7.19 (s, 1H), 6.98 (m, 1H), 6.50 (m, 1H), 4.36 (m, 1H), 3.65-3.40 (m, 6H), 2.90-2.78 (m, 4H), 1.97-1.79 (m, 1H), 1.56 ppm (s, 9H). MW=531 confirmed by LC-MS, tᵣ=11.24 min (Method Y1) MH⁺=529-533.

### Synthesis of tert-Butyl 2-(6,6-Dichloro-4-(2-(3-(2,6-dichlorophenyl)isoxazol-5-yl)pyridin-4-yl)-5-oxohexanoyl)pyrrolidine-1-carboxylate

tert-Butyl 2-(3-(2-(3-(2,6-dichlorophenyl)isoxazol-5-yl)pyridin-4-ylamino)propanoyl)pyrrolidine-1-carboxylate (125 mg, 0.24 mmol) was dissolved in anhydrous dichloromethane (3 mL) with triethylamine (0.40 mL, 0.29 mmol). The mixture was cooled in an ice-bath under nitrogen, then a solution of dichloroacetyl chloride (0.28 mL, 0.29 mmol) in anhydrous dichloromethane (1 mL) was added dropwise. After the addition was completed the ice-bath was removed and the mixture was stirred at room temperature for 2 h. The reaction mixture was diluted with dichloromethane and then washed successively with water, 10% hydrochloric acid and saturated sodium bicarbonate solution. The organic solution was dried over anhydrous sodium sulfate, filtered and concentrated under reduced pressure. The resulting residue was purified by flash column chromatography, on silica gel, eluting with 30:70 ethyl acetate:hexanes to give tert-butyl 2-(6,6-dichloro-4-(2-(3-(2,6-dichlorophenyl)isoxazol-5-yl)pyridin-4 yl)-5-oxohexanoyl)pyrrolidine-1-carboxylate (62 mg) as a yellow oil. ¹H NMR (CDCl₃): 8.80 (m, 1H), 7.83 (s, 1H), 7.48 (m, 2H), 7.37 (m, 1H), 5.98 (br s, 1H), 4.26 (m, 1H), 4.10 (m, 2H), 3.59-3.39 (m, 2H), 2.96 (m, 2H), 2.19 (m, 1H), 1.84 (m, 1H), 1.41 ppm (s, 9H). MW=642 confirmed by LC-MS, tᵣ=14.49 min (Method Y1) MH⁺=640-644.

### Synthesis of 2,2-Dichloro-N-(2-(3-(2,6-dichlorophenyl)isoxazol-5-yl)pyridin-4-yl)-N-(3-oxo-3-(pyrrolidin-2-yl)propyl)acetamide (Cpd. 1024)

Trifluoroacetic acid (0.5 mL) was added to a solution of tert-butyl 2-(3-(2,2-dichloro-N-(2-(3-(2,6-dichlorophenyl)isoxazol-5-yl)pyridinA-yl)acetamido)propanoyl)pyrrolidine-1-carboxylate (62 mg, 0.10 mmol) in dichloromethane (2 mL) at 0° C. The resulting mixture was allowed to stir at 0° C. for 2 h, then concentrated under reduced pressure. The residue was dissolved in dichloromethane (2 mL) and concentrated under reduced pressure, twice more. Lyophilization produced 2,2-dichloro-N-(2-(3-(2,6-dichlorophenyl)isoxazol-5-yl)pyridin-4-yl)43-oxo-3Apyrrolidin-2-yl)propyl)acetamide (Cpd. 1024, 63 mg) as a white solid. ¹H NMR (CDCl₃): 8.91 (d, 1H), 7.98 (s, 1H), 7.50-7.37 (m, 4H), 7.12 (s, 1H), 6.08 (br s, 1H), 5.91 (s, 1H), 4.76 (m, 1H), 4.24-4.02 (m, 2H), 3.49 (m, 2H), 3.09 (m, 2H), 2.56 (m, 1H), 2.06 ppm (m, 3H). MW=542 confirmed by LC-MS, tᵣ=10.47 min (Method Y1) MH⁺=540-544.

### Synthesis of 2,2-Dichloro-N-(2-(3-(2,6-dichlorophenyl)isoxazol-5-yl)pyridin-4-yl)-N-((2-(pyridin-2-yl)ethoxy)methyl)acetamide (Cpd. 1028)

### Synthesis of 2-(3-(2,6-Dichlorophenyl)isoxazol-5-yl)-N-((2-(pyridin-2-yl)ethoxy)methyl)pyridin-4-amine

Sodium hydride (120 mg, 2.9 mmol) was added slowly to a solution of 2-(2-hydroxyethyl)pyridine (0.33 mL, 0.58 mmol) in anhydrous tetrahydrofuran (20 mL) at 0° C. Once the evolution of hydrogen ceased 2-(3-(2,6-dichlorophenyl)isoxazol-5-yl)pyridin4-amine hydrochloride (200 mg, 0.58 mmol) and paraformaldehyde (147 mg, 1.64 mmol) added. The resulting mixture was stirred at room temperature for 5 h and hydrolyzed with ice-cooled water and extracted with ethyl acetate. The organic extracts were washed with water, then dried over anhydrous sodium sulfate, filtered and concentrated under reduced pressure to yield 2-(3-(2,6-dichlorophenyl)isoxazol-5-yl)-N-((2-(pyridin-2-yl)ethoxy)methyl)pyridin4-amine. The product was carried forward without further purification.

### Synthesis of 2,2-Dichloro-N-(2-(3-(2,6-dichlorophenyl)isoxazol-5-yl)pyridin-4-yl)-N-((2-(pyridin-2-yl)ethoxy)methyl)acetamide (Cpd. 1028) (FIG. 27)

2-(3-(2,6-Dichlorophenyl)isoxazol-5-yl)-N-((2-(pyridin-2-yl)ethoxy)methyl)pyridin-amine (100 mg, 0.24 mmol) was dissolved in anhydrous dichloromethane with triethylamine (0.4 mL, 0.29 mmol). The mixture was cooled in an ice-bath under nitrogen, then a solution of dichloroacetyl chloride (0.3 mL, 0.29 mmol) was added dropwise. After the addition was completed the ice-bath was removed and the mixture was stirred at room temperature overnight. The reaction mixture was diluted with dichloromethane and then washed successively with water, 10% hydrochloric acid and saturated sodium bicarbonate solution. The organic solution was dried over anhydrous sodium sulfate, filtered and concentrated under reduced pressure. The resulting residue was purified by flash column chromatography, on silica gel, eluting with 99:1 dichloromethane:methanol to yield 2,2-dichloro-N-(2-(3-(2,6-dichlorophenyl)isoxazol-5-yl)pyridin-4-yl-N-((2-(pyridin-2-yl)ethoxy)methyl)acetamide (Cpd. 1028). ¹H NMR (CDCl₃): 8.7 (d, 1H), 8.6 (d, 1H), 7.8 (s, 1H), 7.4 (m, 2H), 7.2-7.1 (m, 3H), 7.0 (s, 1H), 6.0 (s, 1H), 5.1 (m, 1H), 5.0 (s, 2H), 4.1 (m, 2H), 3.1 ppm (m, 2H). MW=636 confirmed by LC-MS, tᵣ=16.41 min (Method Y1) MH⁺=634-638.

### Synthesis of 2,2-Dichloro-N-(3-(3-(2-chloro-6-(piperidin-4-yloxy)phenyl)isoxazol-5-yl)phenyl)-N-((5-isopropyl-2-oxo-1,3-dioxol-4-yl)methyl)acetamide (See FIG. 6) (Cpd. 1034)

### Synthesis of 4-((3-Ethynylphenylamino)methyl)-5-isopropyl-1,3-dioxol-2-one

3-Ethynylaniline (904 μL, 8.7 mmol) and 44bromomethyl)-5-isopropyl-1,3-dioxol-2-one (2.0 g, 9.09 mmol) were dissolved in acetonitrile (20 mL). Sodium bicarbonate (873 mg, 10.4 mmol) was added and the reaction mixture was heated at reflux under a nitrogen atmosphere overnight. The mixture was then concentrated under reduced pressure, extracted with ethyl acetate and washed with water. The organic layer was then dried over anhydrous sodium sulfate and concentrated under reduced pressure. Purification by column chromatography, on silica gel, eluting with 1:9 ethyl acetate:hexanes yielded 44(3-ethynylphenylamino)methyl]5-isopropyl-1,3-dioxol-2-one (570 mg) as a pale yellow foam. ¹H NMR (CDCl₃): 7.15 (t, 1H), 6.95 (m, 1H), 6.75 (m, 1H), 6.60 (m, 1H), 4.10 (s, 2H), 3.05 (s, 1H), 2.90 (s, 1H), 1.25 ppm (s, 6H). MW=257 confirmed by LC-MS, tᵣ=13.66 min (Method Y1) MH⁺=258.

### Synthesis of 2,2-Dichloro-N-(3-ethynylphenyl)-N-((5-isopropyl-2-oxo-1,3-dioxol-4-yl)methyl)acetamide (FIG. 32)

4-((3-Ethynylphenylamino)methyl)-5-isopropyl-1,3-dioxol-2-one (540 mg, 2.1 mmol) was dissolved in anhydrous dichloromethane (20 mL) with diisopropylethylamine (0.47 mL, 2.5 mmol). The mixture was cooled in an ice-bath under nitrogen, then a solution of dichloroacetyl chloride (0.24 mL, 2.5 mmol) in anhydrous dichloromethane (5 mL) was added dropwise. After the addition was completed the ice-bath was removed and the mixture was stirred at room temperature overnight. The reaction mixture was diluted with dichloromethane and then washed successively with water and brine. The organic solution was dried over anhydrous sodium sulfate, filtered, and concentrated under reduced pressure. The resulting residue was purified by column chromatography, on silica gel, eluting with 1:9 ethyl acetate:hexanes to give 2,2-dichloro-N-(3-ethynylphenyl)-4(5-isopropyl-2-oxo-1,3-dioxol4-yl)methyl)acetamide (508 mg) as a beige solid. MW=368 confirmed by LC-MS, tᵣ=14.11 min (Method Y1) MH⁺=366-370.

### Synthesis of tert-Butyl 4-(Tosyloxy)piperidine-1-carboxylate

1-tert-Butoxycarbonyl-4-hydroxypiperidine (2 g, 9.95 mmol), ptoluenesulfonyl chloride (2.08 g, 10.9 mmol), triethylamine (1.6 mL, 11.9 mmol) and 4-dimethylaminopyridine (61 mg, 0.5 mmol) were dissolved in dichloromethane (50 mL). The resulting mixture was allowed to stir at room temperature overnight. The reaction mixture was diluted with dichloromethane and then washed successively with water, saturated sodium bicarbonate solution, and brine. The organic solution was dried over anhydrous sodium sulfate, filtered and concentrated under reduced pressure. The resulting residue was purified by column chromatography, on silica gel, eluting with 1:9 ethyl acetate:hexanes to give tert-butyl 44tosyloxy)piperidine-1-carboxylate (1.42 g) as white solid. ¹H NMR (CDCl₃): 7.80 (d, 2H), 7.35 (d, 2H), 4.65 (m, 1H), 3.60 (m, 2H), 3.25 (m, 2H), 2.45 (s, 3H), 1.75 (m, 4H), 1.45 ppm (s, 9H).

### Synthesis of tert-Butyl 4-(3-Chloro-2-formylphenoxy)piperidine-1-carboxylate

tert-Butyl 4-(tosyloxy)piperidine-1-carboxylate (1.42 g, 4.0 mmol) and 2-chloro-6-hydroxybenzaldehyde (620 mg, 3.3 mmol) were dissolved in N,N-dimethylformamide (20 mL) and potassium carbonate (552 mg, 4.0 mmol ) was added. The reaction mixture was allowed to stir at 60° C. overnight. Ice was added and the mixture was acidified with 6N hydrochloric acid. The mixture was extracted with ethyl acetate and the organic layer was washed with brine. The organic solution was dried over anhydrous sodium sulfate, filtered, and concentrated under reduced pressure. The resulting residue was purified by column chromatography, on silica gel, eluting with 2:8 ethyl acetate:hexanes to give tertbutyl 4-(3-chloro-2-formylphenoxy)piperidine-1-carboxylate (430 mg) as a white solid. ¹H NMR (CDCl₃): 10.50 (s, 1H), 7.35 (t, 1H), 7.05 (d, 1H), 6.90 (d, 1H), 4.60 (m, 1H), 3.65 (m, 2H), 3.45 (m, 2H), 1.85 (m, 4H),1.40 ppm (s, 9H).

### Synthesis of (E)-tert-Butyl 4-(3-Chloro-2-((hydroxyimino)methyl)phenoxy)piperidine-1-carboxylate

tert-Butyl 43-chloro-2-formylphenoxy)piperidine-1-carboxylate (430 mg, 1.3 mmol) and hydroxylamine hydrochloride (97 mg, 1.4 mmol) were dissolved in pyridine (15 mL) and allowed to stir at room temperature overnight. The mixture was then concentrated under reduced pressure to yield (E~tertbutyl 443-chloro-24(hydroxyimino)methyl)phenoxy)piperidine-1-carboxylate (450 mg) as a light yellow oil. MW=354 confirmed by LC-MS, tᵣ=17.67 min (Method Y1) MH⁺=353-355

### Synthesis of tert-Butyl 4-(3-Chloro-2-(5-(3-(2,2-dichloro-N-((5-isopropyl-2-oxo-1,3-dioxol-4-yl)methyl)acetamido)phenyl)isoxazol-3-yl)phenoxy)piperidine-1-carboxylate

(E)-tert-Butyl 4-(3-chloro-2-((hydroxyimino)methyl)phenoxy)piperidine-1-carboxylate (200 mg, 0.56 mmol), 2,2-dichloro-NA3-ethynylphenyl]-N4(5-isopropyl-2-oxo-1,3-dioxol4-yl)methyl)acetamide (180 mg, 0.49 mmol), chloramine-T (140 mg, 0.62 mmol), and ethanol (2 mL) were placed in a sealed microwave vial. The reaction mixture was heated at 100° C. for 900 seconds. Once the reaction was complete the reaction mixture was concentrated under reduced pressure, extracted with ethyl acetate, and washed with cold 1N sodium hydroxide, water and brine. The organic solution was dried over anhydrous sodium sulfate, filtered, and concentrated under reduced pressure. The resulting residue was purified by column chromatography, on silica gel, eluting with 2:8 ethyl acetate:hexanes to give tert-butyl 4-(3-chloro-2-(5-(3-(2,2-dichloro-N-((5-isopropyl-2-oxo-1,3-dioxol4-yl)methyl)acetamido)phenyl)isoxazol-3-yl)phenoxy)piperidine-1-carboxylate (110 mg) as a white solid. ¹H NMR (CDCl₃): 7.95 (m, 1H), 7.75 (m, 1H), 7.65 (t, 1H), 7.40 (m, 2H), 7.15 (d, 1H), 6.90 (d, 1H), 6.75 (s, 1H), 5.85 (s, 1H), 4.60 (br s, 2H), 3.40 (m, 4H), 1.80 (m, 4H), 1.55 (s, 9H), 1.25 ppm (m, 6H). MW=721 confirmed by LC-MS, tᵣ=17.60 min (Method Y1) MH⁺=719-723

### Synthesis of 2,2-Dichloro-N-(3-(3-(2-chloro-6-(piperidin-4-yloxy)phenyl)isoxazol-5-yl)phenyl)-N-((5-isopropyl-2-oxo-1,3-dioxol-4-yl)methyl)acetamide (See FIG. 32) (Cpd. 1034)

tert-Butyl 4-(3-chloro-2-(5-(3-(2,2-dichloroN4(5-isopropyl-2-oxo-1,3-dioxol-5-yl)methyl)acetamido)phenyl)isoxazol-3-yl)phenoxy)piperidine-1-carboxylate (100 mg, 0.14 mmol) was dissolved in dichloromethane (500 gL). The solution was cooled in an ice-bath and then a cooled solution of trifluoroacetic acid (3 mL) in dichloromethane (3 mL) was added dropwise. The reaction mixture was allowed to stir at 0° C. for 3 h. The reaction mixture was diluted with dichloromethane, washed with water, and dried over anhydrous sodium sulfate, filtered, and concentrated under reduced pressure to yield 2,2-dichloro-N4-(4-(4-(2-chloro-6-(piperidin-4-yloxy)phenyl)isoxazol-5-yl)phenyl)-N-((5-isopropyl-2-oxo-1,3-dioxol-4-yl)methyl)acetamide (Cpd. 1034, 60 mg) as a pale yellow foam. ¹H NMR (CDCl₃): 7.95 (m, 1H), 7.85 (m, 1H), 7.70 (t, 1H), 7.45 (m, 2H), 7.15 (m, 1H), 6.95 (m, 1H), 5.85 (s, 1H), 4.80 (br s, 2H), 3.15 (m, 1H), 1.20 ppm (m, 6H). MW=621 confirmed by LC-MS, tᵣ=11.60 min (Method Y1) MH⁺=619-623.

### Synthesis of 2,2-Dichloro-N-(2-(3-(2,6-Dichlorophenyl)isoxazol-5-yl)pyridin-4-yl)-N-((1-methyl-1H-imidazol-2-yl)methyl)acetamide (Cpd. 1037)

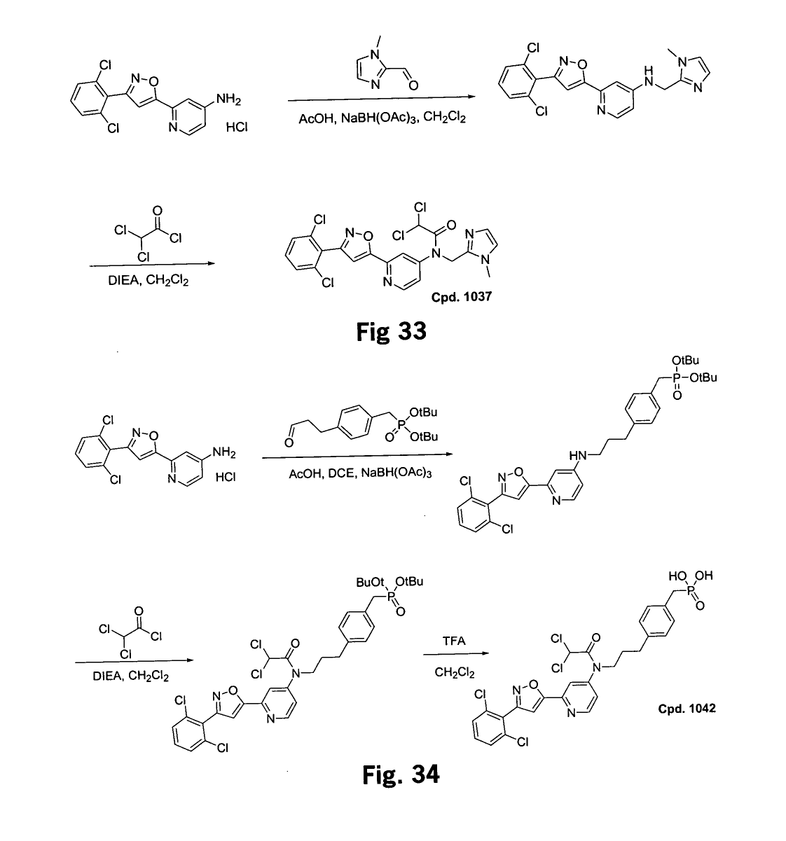

### Synthesis of 2-(3-(2,6-Dichlorophenyl)isoxazol-5-yl)-N-((1-methyl-1H-imidazol-2-yl)methyl)pyridin-4-amine (FIG. 33)

2-3-2,6-Dichlorophenyl)isoxazol-5-yl)pyridin-4-amine hydrochloride (300 mg, 0.88 mmol), 1-methyl-2-imidazolecarboxaldehyde (193 mg, 1.75 mmol), sodium triacetoxyborohydride (187 mg, 1.75 mmol), and acetic acid (53 mg, 0.88 mmol) were combined in dichloromethane (10 mL). The resulting mixture was stirred at room temperature overnight. The solution was then washed with water, dried over anhydrous sodium sulfate, filtered and concentrated under reduced pressure. The resulting residue was purified by flash column chromatography, on silica gel, eluting with 98:2 dichloromethane:methanol to yield 2-(3-(2,6-dichlorophenyl)isoxazol-5-yl)-N-((1-methyl-1H-imidazol-2-yl)methyl)pyridin4-amine (50 mg) as a white solid. ¹H NMR (CDCl₃): 8.3 (m, 1H), 7.4 (m, 3H), 7.0 (m, 2H), 6.9 (s, 1H), 6.7 (m,1 H), 6.5 (br s, 1H), 4.5 (m, 2H), 3.7 ppm (s, 3H). MW=400 confirmed by LC-MS, tᵣ=2.17 min (Method B) MH⁺=398-402.

### Synthesis of 2,2-Dichloro-N-(2-(3-(2,6-Dichlorophenyl)isoxazol-5-yl)pyridin-4-yl)-N-((1-methyl-1H-imidazol-2-yl)methyl)acetamide (Cpd. 1037)

2-3-(2,6-Dichlorophenyl)isoxazol-5-yl-N-((1-methyl-i H-imidazol-2-yl)methyl)pyridin-4-amine (40 mg, 0.lmmol) was dissolved in anhydrous dichloromethane (10 mL) with diisopropylethylamine (21 μL, 0.12 mmol). The mixture was cooled in an ice-bath, then a solution of dichloroacetyl chloride (12 μL, 0.12 mmol) in anhydrous dichloromethane (0.5 mL) was added dropwise. After the addition was complete the ice-bath was removed and the mixture was stirred at room temperature overnight. The reaction mixture was diluted with dichloromethane and then was washed with water. The organic solution was dried over anhydrous sodium sulfate, filtered and concentrated under reduced pressure. The resulting residue was purified by prep-scale reverse phase high performance liquid chromatography to provide 2,2-dichloro-N-(2-(3-(2,6-dichlorophenyl)isoxazol-5-yl)pyridin4-yl)-N-((1-methyl-1H-imidazol-2-yl)methyl)acetamide (Cpd. 1037, 5 mg), as a white solid. ¹H NMR (CDCl₃): 8.8 (m, 1H), 8.0 (s, 1H), 7.8 (m, 1H), 7.4 (m, 3H), 7.1 (s, 1H), 7.0 (s, 1H), 6.0 (s, 1H), 5.4 (s, 2H), 4.0 ppm (s, 3H). MW=509 confirmed by LC-MS, tᵣ=10.83 min (Method Y1) MH⁺=507-511.

### Synthesis of 4-(3-(2,2-Dichloro-N-(2-(3-(2,6-dichlorophenyl)isoxazol-5-yl)pyridin-4-yl)acetamido)propyl)benzylphosphonic Acid (Cpd. 1042)

### Synthesis of Di-tert-butyl 4-lodobenzylphosphonate (FIG. 34)

Di-tert-butyl phosphite (4.8 g, 24.7 mmol) was dissolved in anhydrous tetrahydrofuran (40 mL). The solution was cooled to −70° C. under argon and then n-butyllithium (17 mL, 1.6 M soln in hexanes, 27.2 mmol) was added dropwise. The mixture stirred for 20 min at −70° C., then was placed in an icebath for 30 min. 4-lodobenzyl bromide (7.15 g, 24.0 mmol) in anhydrous tetrahydrofuran (20 mL) was added. The reaction was allowed to warm to room temperature while stirring overnight. The reaction was quenched with saturated aqueous ammonium chloride solution, followed by extraction with ethyl acetate. The organic layer was washed with brine, dried over anhydrous sodium sulfate, filtered and concentrated under reduced pressure. The resulting residue was purified by flash column chromatography, on silica gel, eluting with 3:7 ethyl acetate:hexanes to provide di-tert-butyl 4-iodobenzylphosphonate (6.71 g) as a pale yellow solid. ¹H NMR (CDCl₃): 7.58 (m, 2H), 7.01 (m, 2H), 2.96 (d, 2H), 1.42 ppm (s, 18H).

### Synthesis of Di-tert-butyl 4-(3-Oxopropyl)benzylphosphonate

Di-tert-butyl 4-iodobenzylphosphonate (6.71 g, 16.4 mmol) was dissolved in anhydrous N,N-dimethylformamide (50 mL) and treated with palladium (II) acetate (250 mg), tetrabutylammonium bromide (5.4 g, 16.8 mmol), anhydrous molecular sieves (4 Å, 4 g), sodium bicarbonate (3.52 g, 42 mmol) and allyl alcohol (1.73 mL, 34.9 mmol). The reaction mixture was bubbled with argon and then stirred at room temperature for 90 h. The reaction mixture was filtered through a pad of Celite. The filtrate was diluted with water and ether. The layers were separated and the aqueous layer was further extracted with ether. The combined organic layers were washed with brine, dried over anhydrous sodium sulfate, filtered and concentrated under reduced pressure. The resulting residue was purified by flash column chromatography, on silica gel, eluting with 1:9 ethyl acetate:hexanes to provide di-tert-butyl 4-(3-oxopropyl)benzylphoshonate (1.44 g) as a pale yellow oil. ¹H NMR (CDCl₃): 9.79 (s, 1H), 7.17 (m, 2H), 7.08 (m, 2H), 2.97 (m, 2H), 2.78 (m, 2H), 1.42 ppm (s, 18H).

### Synthesis of Di-tert-butyl 4-(3-(2-(3-(2,6-Dichlorophenyl)isoxazol-5-yl)pyridin-4-ylamino)propyl)benzylphosphonate

2-(3-(2,6-Dichlorophenyl)isoxazol-5-yl)pyridin4-amine hydrochloride (200 mg, 0.59 mmol), ditertbutyl 443-oxopropyl)benzylphosphonate (267 mg, 0.79 mmol), sodium triacetoxyborohydride (278 mg, 1.3 mmol), and acetic acid (39 gL , 0.59 mmol) were combined in anhydrous 1,2-dichloroethane (2 mL), and sonicated at room temperature for 3 h. The solution was filtered through a pad of Celite. The Celite was washed with ethyl acetate. The combined filtrates were washed with water, then dried over anhydrous sodium sulfate, filtered and concentrated under reduced pressure. The resulting residue was purified by flash column chromatography, on silica gel, eluting with 98:2 dichloromethane:methanol to yield di-tert-butyl 4-(3-(2-(3-(2,6-dichlorophenyl)isoxazol-5-yl)pyridin4-ylamino)propyl)benzylphosphonate (180 mg) as a white solid. MW=630 confirmed by LC-MS, tᵣ=4.80 min (Method B) MH⁺=628-632.

### Synthesis of Di-tert-butyl 4-(3-(2,2-Dichloro-N-(2-(3-(2,6-dichlorophenyl)isoxazol-5-yl)pyridin-4-yl)acetamido)propyl)benzylphosphonate

Di-tert-butyl 4-(3-(2-(3-(2,6-dichlorophenyl)isoxazol-5-yl)pyridin-4-ylamino)propyl)benzylphosphonate (180 mg, 0.29mmol) was dissolved in anhydrous dichloromethane (20 mL) with diisopropylethylamine (66 μL, 0.34 mmol). The mixture was cooled in an ice-bath, then a solution of dichloroacetyl chloride (37 μL, 0.34 mmol) in dichloromethane (0.5 mL) was added dropwise. After the addition was complete the ice-bath was removed and the mixture was stirred at room temperature overnight. The reaction mixture was diluted with dichloromethane and then washed with water. The organic solution was dried over anhydrous sodium sulfate, filtered and concentrated under reduced pressure. The resulting residue was purified by flash column chromatography, on silica gel, eluting with 98:2 dichloromethane:methanol to provide di-tert-butyl 4-(3-(2,2-dichloro-N-(2-(3-(2,6-dichlorophenyl)isoxazol-5yl)pyridin4-yl)acetamido)propyl)benzylphosphonate (54 mg) as a white solid. ¹H NMR (CDCl₃): 8.8 (m, 1H), 8.0 (s, 1H), 7.9 (m, 1H), 7.4 (m, 3H), 7.2 (m, 2H), 7.1 (s, 1H), 7.0 (m, 2H), 5.9 (s, 1H), 3.9 (m, 2H), 3.0 (m, 2H), 2.6 (m, 2H), 1.9 (m, 2H), 1.4 ppm (s, 18H). MW=741 confirmed by LC-MS, tᵣ=17.50 min (Method Y1) MH⁺=739-743.

### Synthesis of 4-(3-(2,2-Dichloro-N-(2-(3-(2,6-dichlorophenyl)isoxazol-5-yl)pyridin-4-yl)acetamido)propyl)benzylphosphonic Acid (Cpd. 1042)

Trifluoroacetic acid (2.5 mL) was added to a solution of di-tert-butyl 4-(3-(2,2-dichloro-N-(2-(3-(2,6-dichlorophenyl)isoxazol-5yl)pyridinyl)acetamido)propyl)benzylphosphonate (54 mg, 0.07 mmol) in dichloromethane (2.5 mL) at 0° C. The resulting mixture was allowed to stir at 0 OC for 3 hours, then concentrated under reduced pressure. The resulting residue was purified by prep-scale reverse phase high performance liquid chromatography to provide 4-(3-(2,2-dichloro-N-(2-(3-(2,6-dichlorophenyl)isoxazol-5-yl)pyridin-4yl)acetamido)propyl)benzylphosphonic acid (Cpd. 1042, 10 mg) as a white solid. ¹H NMR (CDCl₃): 8.7 (m, 1H), 7.8 (m, 1H), 7.4 (m, 3H), 7.1 (m, 3H), 6.9 (m, 2H), 5.9 (s, 1H), 3.8 (m, 2H), 3.0 (m, 2H), 2.5 (m, 2H), 1.8 ppm (m, 2H). MW=628 confirmed by LC-MS, tᵣ=12.06 min (Method Y1) MH⁺=626-630.

### Synthesis of 2,2-Dichloro-N-(3-(3-(2,6-dichlorophenyl)isoxazol-5-yl)phenyl)-N-((5-((4,5-dihydroxy-6-(hydroxymethyl)-tetrahydro-2H-pyran-2-yloxy)methyl)-2-oxo-1,3-dioxol-4-yl)methyl)acetamide (Cpd. 1044)

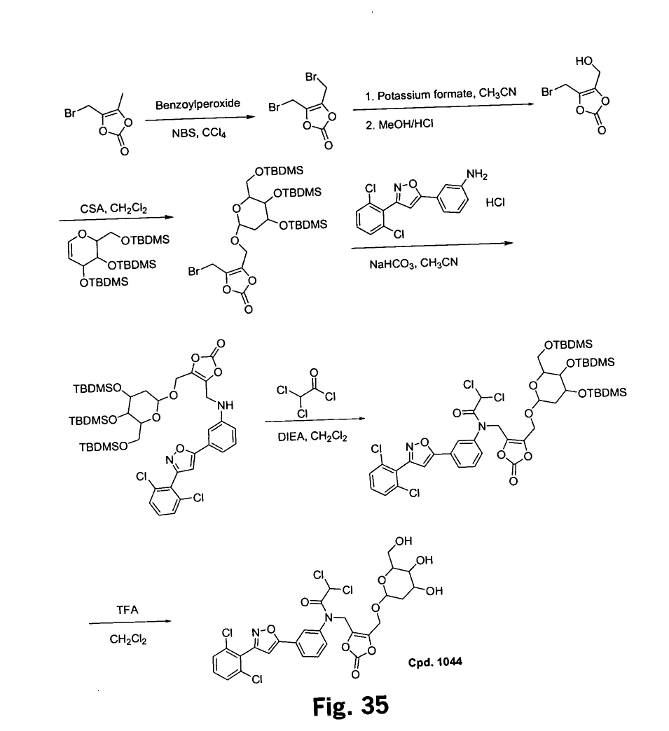

### Synthesis of 4,5-Bis(bromomethyl)-1,3-dioxol-2-one (FIG. 35)

Bromomethyl-1,3-dioxolene (5 g, 25.92 mmol) and N-bromosuccinimide (4.61 g, 25.92 mmol) were dissolved in carbon tetrachloride (250 mL). Benzoylperoxide (250 mg) was added and the mixture was allowed to heat at reflux for 5 h. The reaction mixture was cooled to room temperature and the solids were removed by filtration. The filtrate was concentrated under reduced pressure to give 4,5-bis(bromomethyl)-1,3-dioxol-2one (5.25 g) as a light yellow oil. ¹H NMR (CDCl₃): 4.21 ppm (s, 4H).

### Synthesis of 4-(Bromomethyl)-5-(hydroxymethyl)-1,3-dioxol-2-one

4,5-Bis(bromomethyl)-1,3-dioxol-2-one (4.6 g, 16.9 mmol) was dissolved in acetonitrile (50 mL). Potassium formate (1.42 g, 16.9 mmol) was added to the solution. The mixture was stirred at room temperature for 12 h. The reaction was concentrated under reduced pressure and the crude residue was dissolved in hydrochloric acid/methanol (25 mL) at 0° C. The reaction was allowed to stir at 0° C. for 3 h and then concentrated under reduced pressure. The crude product was purified by column chromatography, on silica gel, eluting with 2:1 hexanes:ethyl acetate to provide 4-(bromomethyl)5-(hydroxymethyl)-1,3-dioxol-2-one (900 mg). ¹H NMR (CDCl₃): 4.51 (s, 2H), 4.30 ppm (s, 2H).

### Synthesis of 4-((4,5-Bis(tert-butyldimethylsilyloxy)-6-((tert-butyldimethylsilyloxy)methyl)-tetrahydro-2H-pyran-2-yloxy)methyl)-5-(bromomethyl)-1,3-dioxol-2-one

4-(Bromomethyl)-5-(hydroxymethyl)-1,3-dioxol-2-one (0.25 g, 1.2 mmol) and tri04tert-butyldimethylsilyl)-D-glucal (1.13 g, 2.3 mmol) were dissolved in anhydrous dichloromethane (10 mL). Camphorsulfonic acid (0.54 g, 2.3 mmol) was added at 0° C. and the reaction mixture was allowed to stir at 0° C. for 3.5 h. The reaction was diluted with dichloromethane (25 mL), washed with water and brine (2×25 mL) and dried over anhydrous magnesium sulfate, filtered, and concentrated under reduced pressure. The resulting residue was purified by flash column chromatography, on silica gel, eluting with 1:15 ethyl acetate:hexanes to provide 4-((4,5-bis(tert-butyldimethylsilyloxy)-6-((tert-butyidimethylsilyloxy)methylptetrahydro-2Hpyran-2-yloxy)methyl)-5-(bromomethyl)-1,3-dioxol-2-one. ¹H NMR (CDCl₃): 4.95 (d, 1H), 4.36 (d, 2H), 4.28 (d, 2H), 4.15 (m, 2H), 3.77-3.56 (m, 3H), 2.08 (m, 2H), 0.98 (s, 27H), 0.18 ppm (d, 18H).

### Synthesis of 4-((4,5-Bis(tert-butyidimethylsilyloxy)-6-((tert-butyldimethylsilyloxy)methyl)-tetrahydro-2H-pyran-2-yloxy)methyl)-5-((3-(3-(2,6-dichlorophenyl)isoxazol-5-yl)phenylamino)methyl)-1,3-dioxol-2-one

4-((4,5-Bis(tert-butyldimethylsilyloxy)-6-((tertbutyldimethylsilyloxy)methyl)-tetrahydro-2H-pyran-2-yloxy)methyl54bromomethyl)-1,3-dioxol-2-one (0.64 g, 0.92 mmol), 706 aniline (0.28g, 0.92 mmol) and sodium bicarbonate (90 mg, 1.11 mmol) were dissolved in anhydrous acetonitrile (25 mL). The above reaction mixture was heated at reflux under an argon atmosphere for 2 d. The reaction mixture was cooled to room temperature and concentrated reduced pressure. The crude product was purified by flash column chromatography, on silica gel, eluting with 1:5 ethyl acetate:hexanes to provide 4-((4,5-bis(tert-butyldimethylsilyloxy)-6-((tert-butyldimethylsilyloxy)methyl)-tetrahydro-2H-pyran-2-yloxy)methyd 54(3-(3-(2,6-dichlorophenyl)isoxazol-5-yl)phenylamino)methylS1,3-dioxol-2-one. ¹H NMR (CDCl₃): 7.90 (s, 1H), 7.75 (m, 1H), 7.66 (m, 1H), 7.45-7.30 (m, 4H), 6.77 (s, 1H), 4.92 (m, 1H), 4.33 (m, 2H), 4.18 (m, 2H), 4.05 (m, 2H), 3.80-3.61 (m, 4H), 2.15 (m, 2H), 0.95 (s, 27H), 0.14 ppm (d, 18H). MW=922 confirmed by LC-MS, tᵣ=14.99 min (Method Y1) MH⁺=920-924.

### Synthesis of N-((5-((4,5-Bis(tert-butyldimethylsilyloxy)-6-((tert-butyidimethylsilyloxy)methyl)-tetrahydro-2H-pyran-2-yloxy)methyl)-2-oxo-1,3-dioxol-4-yl)methyl)-2,2-dichloro-N-(3-(3-(2,6-dichlorophenyl)isoxazol-5-yl)phenyl)acetamide

4-((4,5-Bis(tert-butyidimethylsilyloxy)-6-((tertbutyldimethylsilyloxy)methyl)-tetrahydro-2H-pyran-2yloxy)methyl)-5-((3-(3-(2,6-dichlorophenyl)isoxazol-5-yl)phenylamino)methyl)-1,3-dioxol-2-one (100 mg, 0.11 mmol), diisopropylethylamine (250 [symbol] L, 0.14 mmol) and dichloroacetyl chloride (120 [symbol] L, 0.12 mmol) were dissolved in anhydrous dichloromethane (10 mL) at 0° C. The reaction mixture was allowed to stir at 0° C. for 3 h under argon. The reaction mixture was diluted with dichloromethane (25 mL) and then washed with water (10 mL) and brine (2×10 mL). The organic solution was dried over anhydrous magnesium sulfate, filtered and concentrated under reduced pressure. The resulting residue was purified by flash column chromatography, on silica gel, eluting with 1:7 ethyl acetate:hexanes to provide N-((5-((4,5-bis(tert-butyldimethylsilyloxy)-6-((tert-butydimethylsilyloxy)methyl)-tetrahydro-2H-pyran-2-yloxy)methyl)-2-oxo-1,3-dioxol4-yl)methyl)-2,2-dichloro-N-(3-(3-(2,6-dichlorophenyl)isoxazol-5-yl)phenyl)acetamide. ¹H NMR (CDCl₃): 7.82 (s, 1H), 7.65 (m, 1H), 7.55 (m, 1H), 7.45-7.30 (m, 4H), 6.60 (s, 1H), 5.85 (s, 1H), 4.98 (m, 1H), 4.28 (m, 2H), 4.08 (m, 2H), 3.92 (m, 2H), 3.78-3.55 (m, 3H), 2.18 (m, 2H), 0.84 (s, 27H), 0.19 ppm (d, 18H). MW=1033 confirmed by LC-MS, tᵣ=16.78 min (Method Y1) MH⁺=1031-1035.

### Synthesis of 2,2-Dichloro-N-(3-(3-(2,6-dichlorophenyl)isoxazol-5-yl)phenyl)-N-((5-((4,5-dihydroxy-6-(hydroxymethyl)-tetrahydro-2H-pyran-2-yloxy)methyl)-2-oxo-1,3-dioxol-4-yl)methyl)acetamide (Cpd. 1044)

N-((5-((4,5-Bis(tertbutyidimethylsilyloxy)-6-((tert-butyldimethylsilyloxy)methyl)-tetrahydro-2H-pyran-2-yloxy)methyl-2-oxo-1,3-dioxol-4-yl)methyl)-2,2-dichloro-N-(3-(3-(2,6-dichlorophenyl)isoxazol-5-yl)phenyl)acetamide (100 mg, 0.10 mmol) and 50% TFA (0.25 mL) were dissolved in anhydrous dichloromethane (5 mL) at 0° C. The reaction mixture was allowed to stir at 0° C. for 1 h. After the removal of the solvents the crude residue was purified by silica gel chromatography (CH₂Cl₂/MeOH=9/1) to obtain 2,2-dichloro-N-(3-(3-(2,6-dichlorophenyl)isoxazol-5-yl)phenyl)-N-((5-((4,5-dihydroxy-6-(hydroxymethylptetrahydro-2H-pyran-2-yloxy)methyl)2-oxo-1,3-dioxol-4-yl)methyl)acetamidein. ¹H NMR (CD₃OD): 7.81 (s, 1H), 7.60 (m, 1H), 7.58 (m, 1H), 7.48-7.34 (m, 4H), 6.55 (s, 1H), 5.89 (s, 1H), 4.95 (m, 1H), 4.55-4.23 (m, 2H), 3.92 (m, 2H), 3.76-3.46 (m, 5H), 2.28 ppm (m, 2H). MW=690 confirmed by LC-MS, tᵣ=10.82 min (Method Y1) MH⁺=688-692.

Cpd. 1008: 1-Adamantyl 2,2-Dichloro-3-(3-(3-(2,6-dichlorophenyl)isoxazol-5-yl)phenylamino)-3-oxopropanoate; MW=594 confirmed by LC-MS, tᵣ=14.77 min (Method A) MH⁺=591-597.

Cpd. 1009: 2, 2-Dichloro-N′-3-(3-(2,6-dichlorophenyl)isoxazol-5-yl)phenyl)-N-3-(3-morpholinopropyl)malonamide; MW=586 confirmed by LC-MS, tᵣ=10.76 min (Method Y1) MH⁺=584-590.

Cpd. 1010: 2,2-Dichloro-N¹-(3-(3-(2,6-dichlorophenyl)isoxazol-5-yl)phenyl)-N³-methylmalonamide; MW=473 confirmed by LC-MS, tᵣ=14.27 min (Method Y1) MH⁺=471-476.

Cpd. 1011: 2,2-Dichloro-N¹-(3-(3-(2,6-dichlorophenyl)isoxazol-5-yl)phenyl)-N³-(pyridin-2-ylmethyl)malonamide; MW=550 confirmed by LC-MS, tᵣ=13.17 min (Method Y1) MH⁺=547-553.

Cpd. 1012: 2,2-Dichloro-N¹-(3-(3-(2,6-dichlorophenyl)isoxazol-5-yl)phenyl)-N³-(2-hydroxyethyl)malonamide; MW=502 confirmed by LC-MS, tᵣ=13.17 min (Method Y1) MH⁺=499-505.

Cpd. 1013: Propyl 4-((2,2-Dichloro-N-(3-(3-(2,6-dichlorophenyl)isoxazol-5-yl)phenyl)acetamido)methyl)phenylcarbamate; MW=607 confirmed by LC-MS, tᵣ=16.90 min (Method Y1) MH⁺=604-610.

Cpd. 1014: 2,2-Dichloro-N-(3-(3-(2,6-dichlorophenyl)isoxazol-5-yl)phenyl)-N-((2-oxo-5-(pyrrolidin-2-yl1,3-dioxol-4-yl)methyl)acetamide; MW=583 confirmed by LC-MS, tᵣ=11.24 min (Method Y1) MH⁺=580-586.

Cpd. 1015: 2,2-Dichloro-N-(3-(3-(2,6-dichlorophenyl)isoxazol-5-yl)phenyl)-N-((2-oxo-5-(piperidin-3-yl1,3-dioxol-4-yl)methyl)acetamide; MW=597 confirmed by LC-MS, tᵣ=10.60 min (Method Y1) MH⁺=594-600.

Cpd. 1016: 2,2-Dichloro-N-(3-(3-(2,6-dichlorophenyl)isoxazol-5-yl)phenyl)-N-((5-neopentyl-2-oxo-1,3-dioxol4-yl)methyl)acetamide; MW=582 confirmed by LC-MS, tᵣ=13.08 min (Method A) MH⁺=579-585.

Cpd. 1017: 2,2-Dichloro-N-((5-cyclobutyl-2-oxo-1,3-dioxol-4-yl)methyl)-N-(3-(3-(2,6-dichlorophenyl)isoxazol-5-yl)phenyl)acetamide; MW=568 confirmed by LC-MS, tᵣ=12.77 min (Method A) MH⁺=565-571.

Cpd. 1018: 2,2-Dichloro-N-(2-(3-(2,6-dichlorophenyl)isoxazol-5-yl)pyridin-4-yl)-N-(3-oxo-3-(piperidin4-yl)propyl)acetamide; MW=556 confirmed by LC-MS, tᵣ=9.99 min (Method Y1) MH⁺=553-559.

Cpd. 1019: tert-Butyl 2,2-Dichloro-3-(3-(3-(2-chloro-6-methoxyphenyl)isoxazol-5-yl)phenylamino)-3-oxopropanoate; MW=512 confirmed by LC-MS, tᵣ=17.02 min (Method Y1) MH⁺=510-514.

Cpd. 1020: Isopropyl 2,2-Dichloro-3-(3-(3-(2-chloro-6-methoxyphenyl)isoxazol-5-yl)phenylamino)-3-oxopropanoate; MW=498 confirmed by LC-MS, tᵣ=16.55 min (Method Y1) MH⁺=496-500.

Cpd. 1021: tert-Butyl 4-((2,2-Dichloro-N-(2-(3-(2,6-dichlorophenyl)isoxazol-5-yl)pyridin-4-yl)acetamido)methyl)benzoate.

Cpd. 1022: 2,2-Dichloro-N-(2-(3-(2,6-dichlorophenyl)isoxazol-5-yl)pyridin-4-yl)-N-(4-(2-morpholinoethoxy)benzyl)acetamide; MW=636 confirmed by LC-MS, tᵣ=11.79 min (Method Y1) MH⁺=634-638.

Cpd. 1023: 2,2-Dichloro-N-(2-(3-(2,6-dichlorophenyl)isoxazol-5-yl)pyridin-4-yl)-N-(4-(4-ethylpiperazin-1-yl)benzyl)acetamide; MW=619 confirmed by LC-MS, tᵣ=11.75 min (Method Y1) MH⁺=617-621.

Cpd. 1024: 2,2-Dichloro-N-(2-(3-(2,6-dichlorophenyl)isoxazol-5-yl)pyridin-4-yl)-N-(3-oxo-3-(pyrrolidin-2-yl)propyl)acetamide; MW=542 confirmed by LC-MS, tᵣ=10.47 min (Method Y1) MH⁺=540-544.

Cpd. 1025: N-((5-Benzyl-2-oxo-1,3-dioxol4-yl)methyl)-2,2-dichloro-N-(3-(3-(2,6-dichlorophenyl)isoxazol-5-yl)phenyl)acetamide; MW=604 confirmed by LC-MS, tᵣ=16.78 min (Method Y1) MH⁺=602-606.

Cpd. 1026: 2,2-Dichloro-N-(2-(3-(2,6-dichlorophenyl)isoxazol-5-yl)pyridin4-yl)-N-(2-morpholinoethyl)acetamide; MW=530 confirmed by LC-MS, tᵣ=11.16 min (Method Y) MH⁺=528-532.

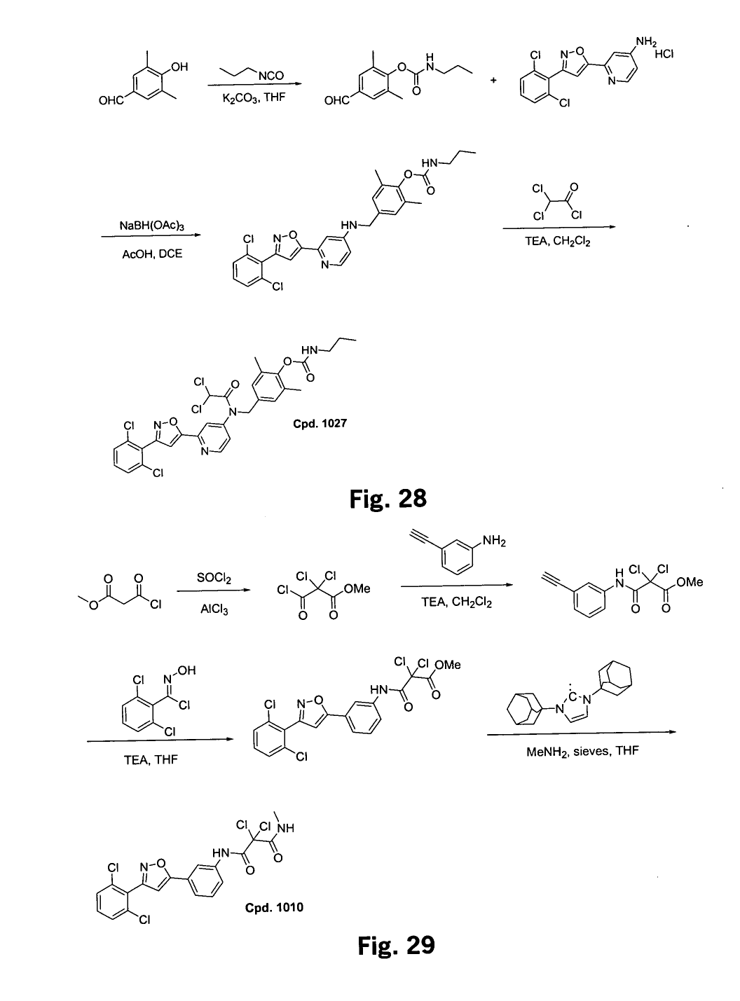

Cpd. 1027: 4-((2, 2-Dichloro-N-(2-(3-(2,6-dichlorophenyl)isoxazol-5-yl)pyridin4-yl)acetamido)methyl)-2,6-dimethylphenyl Propylcarbamate (FIG. 28); MW=636 confirmed by LC-MS, tᵣ=16.41 min (Method Y1) MH⁺=634-638.

Cpd. 1028: 2,2-Dichloro-N-(2-(3-(2,6-dichlorophenyl)isoxazol-5-yl)pyridin-4-yl)-N-((2-(pyridin-2-yl)ethoxy)methyl)acetamide; MW=552 confirmed by LC-MS, tᵣ=12.08 min (Method Y1) MH⁺=550-554.

Cpd. 1029: 3-Chloro-2-(5-(3-(2,2-dichloro-N-((5-isopropyl-2-oxo-1,3-dioxol-4-yl)methyl)acetamido)phenyl)isoxazol-3-yl)benzoic Acid; MW=565 confirmed by LC-MS, tᵣ=15.00 min (Method Y1) MH⁺=563-567.

Cpd. 1030: 2,2-Dichloro-N-(3-(3-(2-cyclopropyl-6-(trifluoromethyl)phenyl)isoxazol-5-yl)phenyl)-N-((5-isopropyl-2-oxo-1,3-dioxol4-yl)methyl)acetamide; MW=595 confirmed by LC-MS, tᵣ=17.15 min (Method Y1) MH⁺=593-597.

Cpd. 1031: 2,2-Dichloro-N-((5-isopropyl-2-oxo-1,3-dioxol-4-yl)methyl)-N-(3-(3-(2-methoxy-6-(trifluoromethyl)phenyl)isoxazol-5-yl)phenyl)acetamide; MW=585 confirmed by LC-MS, tᵣ=15.98 min (Method Y1) MH⁺=583-587.

Cpd. 1032: Methyl 3-Chloro-2-(5-(3-(2,2-dichloro-N-((5-isopropyl-2-oxo-1,3-dioxol4-yl)methyl)acetamido)phenyl)isoxazol-3-yl)benzoate; MW=579 confirmed by LC-MS, tᵣ=15.95 min (Method Y1) MH⁺=577-581.

Cpd. 1033: tert-Butyl 4-(3-Chloro-2-(5-(3-(2,2-dichloro-N-((5-isopropyl-2-oxo-1,3-dioxol-4-yl)methyl)acetamido)phenyl)isoxazol-3-yl)phenoxy)piperidine-carboxylate MW=721 confirmed by LC-MS, tᵣ=17.60 min (Method Y1) MH⁺=719-723.

Cpd. 1034: 2,2-Dichloro-N-(3-(3-(2-chloro-6-(piperidin4-yloxy)phenyl)isoxazol-5-yl)phenyl)-N-((5-isopropyl-2-oxo-1,3-dioxol-4-yl)methyl)acetamide; MW=62 1 confirmed by LC-MS, tᵣ=11.60 min (Method Y1) MH⁺=619-623.

Cpd. 1035: N-(3-(3-(2(1-Acetylpiperidin-4-yloxy)-6-chlorophenyl)isoxazol-5-yl)phenyl)-2,2-dichloro-N-((5-isopropyl-2-oxo-1,3-dioxol4-yl)methyl)acetamide; MW=663 confirmed by LC-MS, tᵣ=14.72 min (Method Y) MH⁺=661-665.

Cpd. 1036: 2,2-Dichloro-N-(2-(3-(2,6-dichlorophenyl)isoxazol-5-yl)pyridin-4-yl-N-((5-neopentyl-2-oxo-1,3-dioxol4-yl)methyl)acetamide; MW=583 confirmed by LC-MS, tᵣ=17.00 min (Method Y1) MH⁺=581-585.

Cpd. 1037: 2,2-Dichloro-N-(2-(3-(2,6-dichlorophenyl)isoxazol-5-yl)pyridin-4-yl)-N-((1-methyl-1H-imidazol-2-yl)methyl)acetamide; MW=511 confirmed by LC-MS, tᵣ=3.69 min (Method B) MH⁺=509-513.

Cpd. 1038: 4-(3-(2,2-Dichloro-N-(2-(3-(2,6-dichlorophenyl)isoxazol-5-yl)pyridin-4-yl)acetamido)propyl)phenyl Diethyl Phosphate; MW=687 confirmed by LC-MS, tᵣ=16.15 min (Method Y1) MH⁺=685-689.

Cpd. 1039: tert-Butyl 4-(4-(3-(2,2-Dichloro-N-(2-(3-(2,6-dichlorophenyl)isoxazol-5-yl)pyridin-4yl)acetamido)propyl)phenyl)piperazine-1-carboxylate; MW=719 confirmed by LC-MS, tᵣ=18.02 min (Method Y1) MH⁺=717-721.

Cpd. 1040: tert-Butyl 4-(3-(2,2-Dichloro-N-(2-(3-(2,6-dichlorophenyl)isoxazol-5-yl)pyridin-4-yl)acetamido)propyl)benzoate; MW=635 confirmed by LC-MS, tᵣ=18.38 min (Method Y1) MH⁺=633-637.

Cpd. 1041: Di-tert-Butyl 4-(3-(2,2-Dichloro-N-(2-(3-(2,6-dichlorophenyl)isoxazol-5-yl)pyridin-4-yl)acetamido)propyl)benzylphosphonate; MW=741 confirmed by LC-MS, tᵣ=17.50 min (Method Y1) MH⁺=739-743.

Cpd. 1042: 4-(3-(2,2-Dichloro-N-(2-(3-(2,6-dichlorophenyl)isoxazol-5-yl)pyridin-4-yl)acetamido)propyl)benzylphosphonic Acid; MW=629 confirmed by LC-MS, tᵣ=12.06 min (Method Y1) MH⁺=627-631.

Cpd. 1043: 2,2-Dichloro-N-(2-(3-(2,6-dichlorophenyl)isoxazol-5-yl)pyridin-4-yl)-N-(3-(4-(piperazin-1-yl)phenyl)propyl)acetamide; MW=629 confirmed by LC-MS, tᵣ=11.73 min (Method Y1) MH⁺=627-631.

Cpd. 1044: N-(2,2-Dichloroacetyl)-3-(3-(2,6-dichlorophenyl)isoxazol-5-yl)-N-((5-((4,5-dihydroxy-6-(hydroxymethyl)-tetrahydro-2H-pyran-2-yloxy)methyl2-oxo-1,3-dioxol-4-yl)methyl)benzamide; MW=690 confirmed by LC-MS, tᵣ=10.82 min (Method Y1) MH⁺=688-692.

### Synthesis of Compound 311a (FIG. 11)

### Synthesis of N-[2-(4-Fluorobenzoyl)ethyl]-3-ethynylaniline (Compound 305a)

3-Ethynylaniline 301a (2.68 g, 22.8 mmol) was heated with 3-chloro4′-fluoropropiophenone 303a (3.86 g, 20.7 mmol) and triethylamine (3.85 mL, 24.7 mmol) in tetrahydrofuran (40 mL) at 65° C. for 14 h. The reaction mixture was concentrated to a small volume, diluted with ethyl acetate and then washed with saturated sodium chloride solution. The organic layer was dried over anhydrous sodium sulfate and concentrated to a small volume. Upon cooling white crystals formed. These were filtered off to give N-[2-(4-fluorobenzoyl)ethyl]-3-ethynylaniline 305a, (1.98 g). ¹H NMR ( CDCl₃) δ 7.94-7.99 (m, 2H), 7.08-7.15 (m, 3H), 6.83-6.87 (m, 1H), 6.76 (m, 1H), 6.61-6.64 (m,lH), 3.61 (t, J=6.0, 2H), 3.25 (t, J=6.0, 2H), 3.02 (s, 1H). MS (m/z): 268 (MH⁺) confirmed by LC-MS, tᵣ=14.36 min (Method Y).

### Synthesis of 2′,2′-Dichloro-N-(3′-ethynylphenyl)-N-[2-(4-fluorobenzoyl)ethyl] Acetamide (Compound 307a)

N-[2-(4-fluorobenzoyl)ethyl]-3-ethynylaniline 305a (1.69 g, 6.25 mmol) was dissolved in dichloromethane (40 mL) with triethylamine (1.14 mL, 1.3 molar equivalents). The solution was cooled on an ice-water bath and then a solution of dichloroacetyl chloride (0.66 mL, 1.1 molar equivalent) in dichloromethane (5 mL) was added dropwise. The reaction mixture was allowed to stir overnight while warming to room temperature. The solution was washed successively with water and saturated sodium bicarbonate solution, then dried over anhydrous sodium sulfate and concentrated under vacuum. Flash column chromatography, on silica gel, eluting with a mixture of 20% ethyl acetate in hexanes gave 2′,2′-dichloro-N-(3′-ethynylphenyl)-N-[2-(4-fluorobenzoyl)ethyl] acetamide 307a as a solid, (400 mg). ¹H NMR ( CDCl₃) δ 7.84-7.94 (m, 2H), 7.51 (m, 1H), 7.42(m, 1H), 7.37 (m, 1H), 7.24-7.27 (m, 1H), 7.04-710 (m, 2H), 5.79 (s, 1H), 4.08 (t, J=7.7, 2H), 3.30 (t, J=7.7, 2H), 3.19 (s, 1H). MS (m/z): 376-380 (MH⁺) confirmed by LC-MS, tᵣ=14.72 min (Method Y).

### Synthesis of 2′,2′-Dichloro-N-[3-[3-(2,6-dichlorophenyl)-5-isoxazolyl]phenyl]-N-[2-(4-fluorobenzoyl)ethyl] Acetamide (Compound 311a)

2′,2′-Dichloro-N-(3′-ethynylphenyl)-N-[2-(4-fluorobenzoyl)ethyl] acetamide 307a (0.40 g, 1.05 mmol) was dissolved in anhydrous tetrahydrofuran (20 mL) and treated with 2,6-dichloro-N-hydroxybenzenecarboximidoyl chloride 309a (0.25 g, 1.12 mmol) and triethylamine (0.20 mL, 1.42 mmol). The solution was heated at reflux for 4.5 h. The reaction was then cooled to room temperature, diluted with ethyl acetate and washed with saturated aqueous sodium chloride solution. The organic layer was dried over anhydrous sodium sulfate and concentrated under vacuum. The residue was purified by flash chromatography, on silica gel, eluting with 20% ethyl acetate in hexanes. 2′,2′-Dichloro-N-[3-[3-(2,6-dichlorophenyl)-5-isoxazolyl]phenyl]-N-[2-(4-fluorobenzoyl)ethyl] acetamide 100a was obtained as a white solid, (165 mg). ¹H NMR (CDCl₃) δ 7.88-7.98 (m, 3H), 7.78 (broad s, 1H), 7.61 (t, J=7.8, 1H), 7.31-7.44 (m, 4H), 7.07-7.13 (m, 2H), 6.69 (s, 1H), 5,88 (s, 1H), 4.18 (t, J=7.3, 2H), 3.38 (t, J=7.3, 2H). MS (m/z): 564-568 (MH⁺) confirmed by LC-MS, tᵣ=12.81 min (Method A).

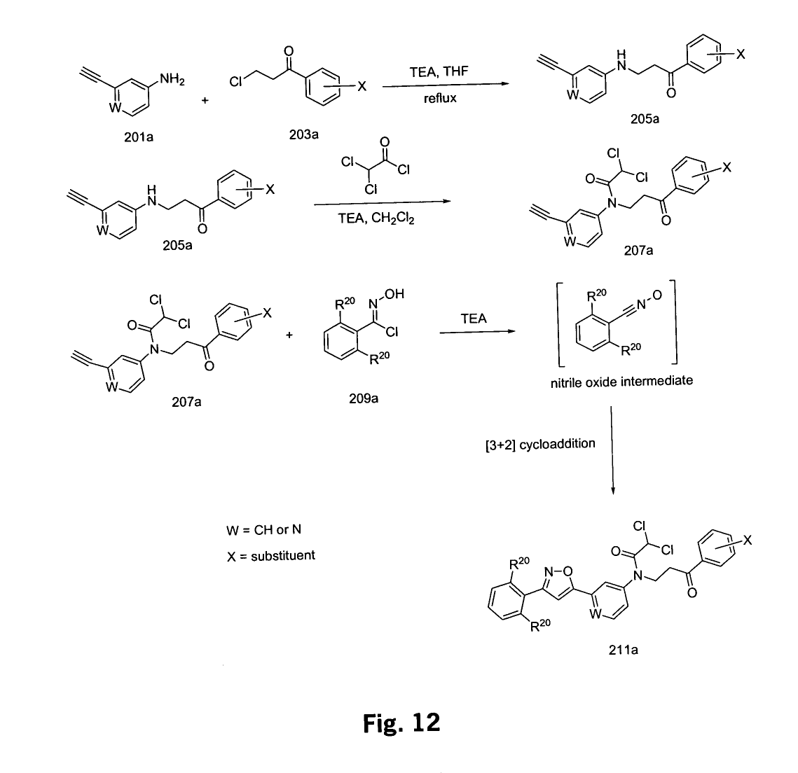

Similarly, various acetamides equivalent to that shown in FIG. 11 can be prepared according to the general reaction sequence depicted in FIG. 12, where W can be CH or N and X is a substituent, including hydrogen, on the aryl group.

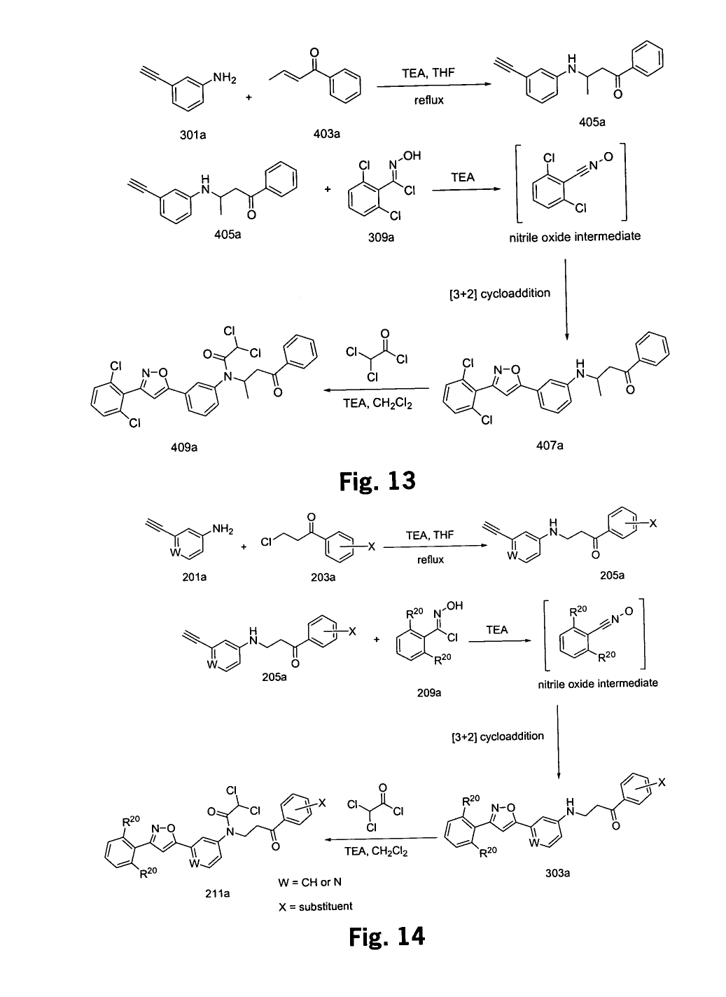

### Synthesis of Compound 407a (FIG. 13)

### Synthesis of N-[2-(3-Benzoyl)propyl]-3-ethynylaniline (Compound 405a)

3-Ethynylaniline 301a (2.03 g, 17.8 mmol) was heated with phenyl propenyl ketone 403a (2.27 g, 15.6 mmol) and triethylamine (2.67 mL,19 mmol) in tetrahydrofuran (40 mL) at 65° C. for 42 h. The reaction mixture was concentrated to a small volume, diluted with ethyl acetate and then washed with saturated sodium chloride solution. The organic layer was dried over anhydrous sodium sulfate and concentrated under vacuum. The residue was purified by flash column chromatography, on silica gel, eluting with 20% ethyl acetate in hexanes to give N-[2-(3-benzoyl)propyl]-3-ethynylaniline 405a as a pale yellow oil, (450 mg). ¹H NMR ( CDCl₃) δ 7.91-7.94 (m, 2H), 7.53-7.57 (m, 1H), 7.42-7.48 (m, 2H), 7.09 (t, J=7.8, 1H), 6.82-6.85 (m, 1H), 6.74 (m, 1H), 6.61 (dd, J=8.0, 2.5, 1H), 4.14 (m, 1H), 3.92 (broad s, 1H), 3.28 ( dd, J=16,4, 4.2, 1H), 3.08 (dd, J=16.5, 7.2, 1H), 3.01 (s, 1H), 1.33 (d, J=6.3, 3H). MS (m/z): 264 (MH⁺) confirmed by LC-MS, tᵣ=14.71 min (Method Y).

### Synthesis of N-[2-(3-Benzoyl)propyl]-3-[3-(2,6-dichlorophenyl)-5-isoxazolyl] Aniline (Compound 407a)

N-[2-(3-Benzoyl)propyl]-3-ethynylaniline 405a (0.45 g, 1.7 mmol) was dissolved in anhydrous tetrahydrofuran (20 mL) and treated with 2,6-dichloro-N-hydroxybenzenecarboximidoyl chloride 309a (0.40 g, 1.8 mmol) and triethylamine (0.31 mL, 2.28 mmol). The solution was heated at reflux for 6 h. The reaction was then cooled to room temperature, diluted with ethyl acetate and washed with saturated aqueous sodium chloride solution. The organic layer was dried over anhydrous sodium sulfate and concentrated under vacuum. The residue was purified by flash column chromatography, on silica gel, eluting with 20% ethyl acetate in hexanes. N-[2-(3-Benzoyl)propyl]-3-[5-[3-(2,6-dichlorophenyl)]isoxazolyl] aniline 407a was obtained as a white solid, (330 mg). ¹H NMR (CDCl₃) δ 7.91-7.94 (m, 2H), 7.54 (m, 1H), 7.39-7.46 (m, 4H), 7.24-7.31 (m, 1H), 7.23 (m, 1H). 7.11-7.16 (m, 2H), 6.71 (m, 1H), 6.55 (s, 1H), 4.21 (m, 1H), 3.32 (dd, J=16.4, 4.4, 1H), 3.12 (dd, J=16.4, 6.9, 1H), 1.35 (d, J=7.3, 3H). MS (m/z): 449-453 (MH⁺) confirmed by LC-MS, tᵣ=17.40 min (Method Y).

### Synthesis of 2′,2′-Dichloro-N-[3-[3-(2,6-dichlorophenyl)-5-isoxazolyl]phenyl]-N-[2-(3-benzoyl)propyl] Acetamide (Compound 407a)

N-[2-(3-Benzoyl)propyl]-3-[3-(2,6-dichlorophenylS5-isoxazolyl] aniline 407a (0.33 g, 0.73 mmol) was dissolved in dichloromethane (10 mL) with triethylamine (0.15 mL). The solution was cooled on an ice-water bath and then a solution of dichloroacetyl chloride (85 μL, 1.2 molar equivalents) in dichloromethane (2 mL) was added dropwise. The reaction mixture was allowed to stir overnight while warming to room temperature. The solution was washed successively with 10% aqueous hydrochloric acid, water and saturated sodium bicarbonate solution, then dried over anhydrous sodium sulfate and concentrated under vacuum. Flash column chromatography, on silica gel, eluting with a mixture of 25% ethyl acetate in hexanes gave 2′,2′-dichloro-N-[3-[3-(2,6-dichlorophenyl)-5-isoxazolyl]phenyl]-N-[2-(3-benzoyl)propyl] acetamide 107a as a white solid, (113 mg). ¹H NMR ( CDCl₃) δ 7.95-7.98 (m, 2H), 7.91 (m, 1H), 7.65(m, 1H), 7.61 (m, 1H), 7.50-7.55 (m, 1H), 7.40-7.47 (m, 4H), 7.36-7.40 (m, 1H), 6.64 (broad s, 1H), 5.72 (broad s, 1H), 4.99 (m, 1H), 3.69 (dd, J=16.2, 7.2, 1H), 3.13 (dd, J=16.2, 6.9, 1H), 1.42 (d, J=6.9, 3H). MS (m/z): 583-587 (MH⁺+Na) confirmed by LC-MS, tᵣ=12.93 min (Method A).

Similarly, various acetamides equivalent to that shown in FIG. 13 can be prepared according to the general reaction sequence depicted in FIG. 14, where W can be CH or N and X is a substituent, including hydrogen, on the aryl group.

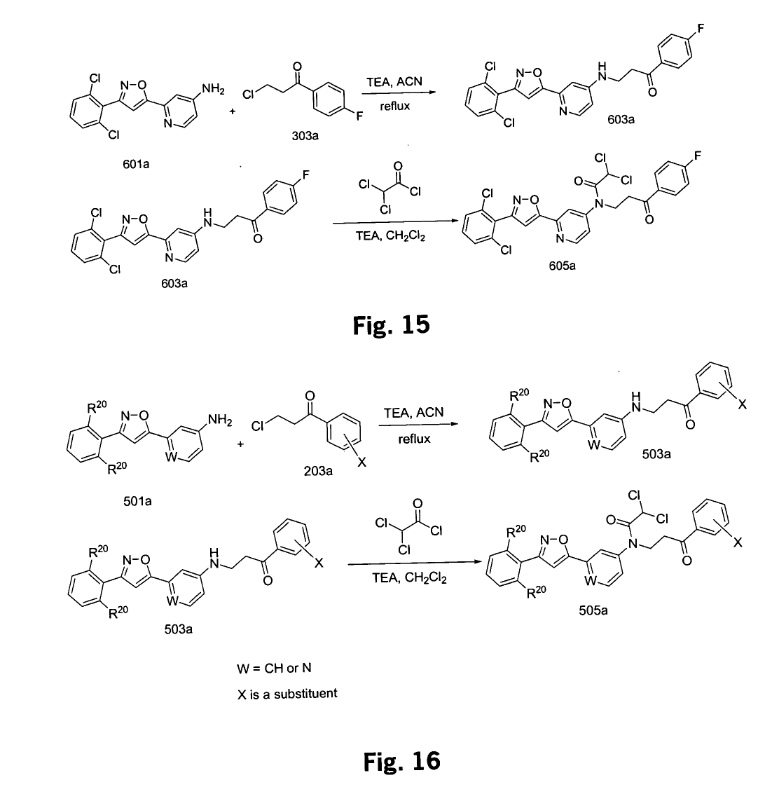

### Synthesis of Compound 605a (FIG. 15)

### Synthesis of 3-(2,6-Dichlorophenyl)-5-[5-[N-[2-(4-fluorobenzoyl)ethyl]amino-2-pyridyl]isoxazole (Compound 603a)

3-Chloro-4′-fluoropropiophenone 303a (220 mg, 1.2 mmol) was added to a mixture of 342,6-dichlorophenyl)-5-(5-amino-2-pyridyl)isoxazole 601a (400 mg, 1.2 mmol) and triethylamine (0.3 mL, 2.4 mmol) in acetonitrile (40 mL). The solution was heated at reflux. After two hours a second portion of 3-chloro-4′-fluoropropiophenone (220 mg, 1.2 mmol) was added, along with a second portion of triethylamine (0.3 mL, 2.4 mmol). After stirring at reflux overnight, the reaction mixture was cooled to room temperature and then washed successively with water, 10% hydrochloric acid and saturated sodium bicarbonate solution. The organic solution was dried over anhydrous sodium sulfate, filtered and concentrated under reduced pressure. The resulting residue was purified by prep-scale reverse phase high performance liquid chromatography to give 603a as a white solid (60 mg, 11%). ¹H NMR (300 MHz, CDCl₃): 8.33 (m, 1H), 8.00 (m, 2H), 7.39 (m, 5H), 7.17 (m, 2H), 6.64 (m, 1H), 3.81 (m, 2H), 3.38 ppm (m, 2H).

### Synthesis of 2′,2′-Dichloro-N-[3-[3-(2,6-dichlorophenyl)-5-isoxazolyi]-(pyridy1)]-N-[2-(4-fluorobenzoyl)ethyl] Acetamide (Compound 605a)

3-(2,6-Dichlorophenyl)-5-[5-[N-[2-(4-fluorobenzoyl)ethyl]amino-2-pyridyl]isoxazole 603a (60 mg, 0.17 mmol) was dissolved in dichloromethane (5 mL) with triethylamine (28 85 μL). The solution was cooled on an ice-water bath and then a solution of dichloroacetyl chloride (20 μL, 1.2 molar equivalents) in dichloromethane (0.5 mL) was added dropwise. The reaction mixture was allowed to stir overnight while warming to room temperature. The solution was washed successively with 10% aqueous hydrochloric acid, water and saturated sodium bicarbonate solution, then dried over anhydrous sodium sulfate and concentrated under vacuum. Purification by flash column chromatography, on silica gel, eluting with a mixture of 25% ethyl acetate in hexanes gave 2′,2′-dichloro-N-[3-[3-(2,6-dichlorophenyl)-5-isoxazolyl]-4-pyridyl)]-N-[2-(4-fluorobenzoyl)ethyl] acetamide 605a as a white solid, (12 mg, 13%). ¹H NMR (300 MHz, CDCl₃): 8.82 (m, 1H), 7.92 (m, 3H), 7.40 (m, 4H), 7.18 (m, 3H), 6.00 (broad s, 1H), 4.27 (m, 2H), 3.43 ppm (m, 2H).

Similarly, various acetamides equivalent to that shown in FIG. 5 can be prepared according to the general reaction sequence depicted in FIG. 6, where W can be CH or N and X is a substituent, including hydrogen, on the aryl group.

The following compounds were prepared by the methods described herein.

Cpd. 311a: 2′,2′-Dichloro-N-[3-[3-(2,6-dichlorophenyl)-5-isoxazolyl]phenyl]-N-[2-(4-fluorobenzoyl)ethyl] Acetamide; MW=566 confirmed by LC-MS, tᵣ=12.81 min (Method A) MH⁺=564-568

Cpd. 102a: 2′,2′-Dichloro-N-[3-[3-(2,6-dichlorophenyl)-5-isoxazolyl]phenyl]-N-[2-(benzoyl)ethyl] Acetamide; MW=548 confirmed by LC-MS, tᵣ=16.82 min (Method Y) MH⁺=546-550

Cpd. 103a: 2′,2′-Dichloro-N-[3-[3-(2,6-dichlorophenyl)-5-isoxazolyl]phenyl]-N-[2-(4-methoxybenzoyl)ethyl] Acetamide; MW=578 confirmed by LC-MS, tᵣ=16.69 min (Method Y) MH⁺=576-580

Cpd. 104a: 2′,2′-Dichloro-N-[3-[3-(2,6-dichlorophenyl)-5-isoxazolyl]phenyl]-N-[2-(4-chlorobenzoyl)ethyl] Acetamide; MW=583 confirmed by LC-MS, tᵣ=13.22 min (Method A) MH⁺=581-585

Cpd. 407a: 2′,2′-Dichloro-N-[3-[3-(2,6-dichlorophenyl)-5-isoxazolyl]phenyl]-N-[2-[3-(benzoyl)propyl] Acetamide; MW=562 confirmed by LC-MS, tᵣ=12.93 min (Method A) M⁺Na=582-586

Cpd. 605a: 2′,2′-Dichloro-N-[3-[3-(2,6-dichlorophenyl)-5-isoxazolyl]-4-(pyridyl)]-N-[2-(4-fluorobenzoyl)ethyl] Acetamide; MW=567 confirmed by LC-MS, tᵣ=16.31 min (Method Y) MH⁺=565-569

Cpd. 127a: 2′,2′-Dichloro-N-[3-[3-[2-chloro-6-(N′-acetyl-4piperdinyloxy)phenyl]-5-isoxazolyl]phenyl]-N-[2-(4-fluorobenzoyl)ethyl] Acetamide; MW=671 confirmed by LC-MS, tᵣ=15.26 min (Method Y) MH⁺=669-673

Cpd. 114a: 2′,2′-Dichloro-N-[3-[3-(2-cyclopropyl-6-trifluoromethylphenyl)-5-isoxazolyl]phenyl]N-[2-(4-fluorobenzoyl)ethyl] Acetamide; MW=605 confirmed by LC-MS, tᵣ=17.26 min (Method Y) MH⁺=603-607

Cpd. 113a: 2′,2′-Dichloro-N-[3-[3-(2-chloro-6-methoxyphenyl)-5-isoxazolyl]phenyl]-N-[2-(4-fluorobenzoyl)ethyl] Acetamide; MW=562 confirmed by LC-MS, tᵣ=16.44 min (Method Y) MH⁺=560-564

Cpd. 128a: 2′,2′-Dichloro-N-[3-[3-(2-chloro-6-tbutoxycarbonylphenyl)-5-isoxazolyl]phenyl]N-[2-(4-fluorobenzoyl)ethyl] Acetamide; MW=632 confirmed by LC-MS, tᵣ=17.65 min (Method Y) MH⁺=630-634

Cpd. 129a: 2′,2′-Dichloro-N-[3-[3-(2-chloro-6-hydroxycarbonylphenyl)-5-isoxazolyl]phenyl]-N-[2-(4-fluorobenzoyl)ethyl] Acetamide; MW=576 confirmed by LC-MS, tᵣ=min (Method ) MH⁺=574-578

Cpd. 133a: 2′,2′-Dichloro-N-[3-[3-(2-chloro-6-methoxycarbonylphenyl)-5-isoxazolyl]phenyl]-N-[2-(4-fluorobenzoyl)ethyl] Acetamide; MW=590 confirmed by LC-MS, tᵣ=min (Method Y) MH⁺=588-592

### Assays For Modulation of HCV

As stated previously, the prodrugs of the invention or the metabolically active agents of the prodrug, A-B-C-NHCOCHX₂, are potent inhibitors of HCV replication and/or proliferation. The activity of the prodrugs of the invention, or their metabolites, can be confirmed in in vitro assays suitable for measuring inhibition of viral or retroviral replication and/or proliferation. The assays may investigate any parameter that is directly or indirectly under the influence of HCV, including, but not limited to, protein-RNA binding, translation, transcription, genome replication, protein processing, viral particle formation, infectivity, viral transduction, etc. Such assays are well-known in the art. Regardless of the parameter being investigated, in one embodiment, to examine the extent of inhibition, samples, cells, tissues, etc. comprising an HCV replicon or HCV RNA are treated with a potential inhibitory prodrug (test compound) and the value for the parameter compared to control cells (untreated or treated with a vehicle or other placebo). Control samples are assigned a relative activity value of 100%. Inhibition is achieved when the activity value of the metabolically active agent of the prodrug relative to the control is about 90%, preferably 50%, and more preferably 25-0%.

Alternatively, the extent of inhibition may be determined based upon the IC₅₀ of the metabolically active agent of the prodrug in the particular assay, as will be described in more detail, below.

In one embodiment, the inhibitory activity of the metabolically active agent of the prodrug can be confirmed in a replicon assay that assesses the ability of a test compound to block or inhibit HCV replication in replicon cells. One example of a suitable replicon assay is the liver cell-line Huh 7-based replicon assay described in Lohmann et al., 1999, Science 285:110-113. A specific example of this replicon assay which utilizes luciferase translation is provided in the Examples Section. In one embodiment of this assay, the amount of test prodrug that yields a 50% reduction in translation as compared to a control cell (IC₅₀) may be determined.

Alternatively, the inhibitory activity of the metabolically active agents of the prodrugs can be confirmed using a quantitative Western immunoblot assay utilizing antibodies specific for HCV non-structural proteins, such as NS3, NS4A NS5A and NS5B. In one embodiment of this assay, replicon cells are treated with varying concentrations of test prodrug to determine the concentration of the metabolically active agent of the test prodrug that yields a 50% reduction in the amount of a non-structural protein produced as compared to a control sample (IC₅₀). A single non-structural protein may be quantified or multiple non-structural proteins may be quantified. Antibodies suitable for carrying out such immunoblot assays are available commercially (e.g., from BIODESIGN International, Saco, Me.).

Alternatively, the inhibitory activity of the metabolically active agent of the prodrugs may be confirmed in an HCV infection assay, such as the HCV infection assay described in Fournier et al., 1998, J. Gen. Virol. 79(10):2367:2374, the disclosure of which is incorporated herein by reference. In one embodiment of this assay, the amount of test prodrug that is metabolized into an active agent that yields a 50% reduction in HCV replication or proliferation as compared to a control cell (IC₅₀) may be determined. The extent of HCV replication may be determined by quantifying the amount of HCV RNA present in HCV infected cells. A specific method for carrying out such an assay is provided in the Examples section.

As yet another example, the inhibitory activity of the metabolically active agent of the prodrugs can be confirmed using an assay that quantifies the amount of HCV RNA transcribed in treated replicon cells using, for example, a Taqman assay (Roche Molecular, Alameda, Calfi.). In one embodiment of this assay, the amount of test prodrug that is metabolized into an active agent that yields a 50% reduction in transcription of one or more HCV RNAs as compared to a control sample (IC₅₀) may be determined.

Regardless of the assay used, metabolically active agents of the prodrugs are generally those which exhibit IC₅₀s in the particular assay in the range of about 1 mM or less. Prodrugs that are metabolized into active agents which exhibit lower IC₅₀s, for example, in the range of about 100 μM, 10 μM, 1 μM, 100 nM, 10 nM, 1 nM, or even lower, are particularly useful for as therapeutics or prophylactics to treat or prevent HCV infections.

### Prodrugs of the Invention Metabolize into Active Agents that Inhibit HCV Translation or Replication

### Replicon Assay

The inhibitory activity of certain prodrugs of the invention, which may inhibit HCV translation or replication prior to metablism and/or are metabolized into active agents, was confirmed using an HCV replicon assay. The HCV replicon can include such features as the HCV 5′ untranslated region including the HCV IRES, the HCV 3′ untranslated region, selected HCV genes encoding HCV polypeptides, selectable markers, and a reporter gene such as luciferase, GFP, etc. In the assay, actively dividing 5-2 Luc replicon-comprising cells (obtained from Ralf Bartenschlager; see Lohmann et al., 1999, Science 285:110-113) were seeded at a density of between about 5,000 and 7,500 cells/well onto 96 well plates (about 90 pi of cells per well) and incubated at 37° C. and 5% CO₂ for 24 hours. Then, the test prodrug (in a volume of about 10 PI) was added to the wells at various concentrations and the cells were incubated for an additional 24-48 hours before luciferase assay. The media was aspirated from each well and Bright-Glo (Promega, Madison, WI) luciferase assay reagents were added to each well according to the manufacturer's instructions. Briefly, the BrightGlo reagent was diluted 1:1 with PBS and 100 l of diluted reagent was added to each well. After 5 min of incubation at room temperature, luciferin emission was quantified with a luminometer. In this assay, the amount of test prodrug that yielded a 50% reduction in luciferase emission (IC₅₀) was determined. This IC₅₀ value may represent the antiviral activity of the prodrug itself, the activity of prodrug transformed into its active metabolized form, or a combination of the two.

### Western Blot Assay

Certain prodrugs of the invention, which may inhibit HCV translation or replication prior to metablism and/or are metabolized into active agents, were also tested for their ability to inhibit HCV replication using a quantitative Western blot analysis with antibodies specific for the HCV NS5A or other non-structural proteins. Actively dividing 9-13 replicon cells were seeded into 6-well plates at a density of 1×10⁵ cells/well in a volume of 2 ml/well and incubated at 37° C. and 5% CO₂ for 24 hours. Various concentrations of test prodrugs (in a volume of 10 ul) were added to the wells and the cells incubated for another 48 hours. Protein samples were prepared from the cultured cells, resolved on a SDS-PAGE gel and transferred to a nitrocellulose membrane. The membrane was blocked with 5% non-fat milk in PBS for 1 hour at room temperature. Primary antibody (anti NS5A antibody; BIODESIGN International, Saco, Me.) incubation was performed for 1 hour at room temperature, after which the membrane was washed 3 times (for 15 min per time) with PBST (PBS plus 0.1% Tween 20). Horseradish peroxidase conjugated secondary antibody incubation was performed for 1 hour at room temperature and the membrane was washed 3 times (for 15 min per time) with PBST. The membrane was then soaked in substrate solution (Pierce) and exposed to a film or quantified using an imager. In this assay, the amount of test prodrug that is believed to be transformed into an active agent under the given conditions that yielded a 50% reduction in the amount of NS5A protein translated as compared to a control sample (IC₅₀) was determined.

The results of the Replicon assays are provided in TABLES 3-12 and 14, below. In TABLES 3 through 12, a value of “+” indicates an IC₅₀ of 10 μM or less in the specified assay; a value of “−” indicates an IC₅₀ of greater than 10 μM in the specified assay. Many of the metabolically active agents of the prodrugs exhibited IC₅₀s in the Replicon assay in the nanomolar range.

TABLE 3

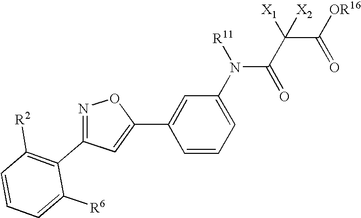

| Cpd | Replicon | X₁ | X₂ | R² | R⁶ | R¹¹ | R¹⁶ |
|---|---|---|---|---|---|---|---|
| 18 | + | Cl | Cl | Cl | Cl | H | -Me |
| 30 | + | Cl | Cl | CF₃ | F | H | -Me |
| 29 | + | Cl | Cl | CF₃ | H | H | -Me |
| 27 | + | Cl | Cl | Cl | OMe | H | -Me |
| 26 | + | Cl | Cl | CF₃ | 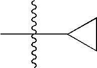 | H | -Me |
| 16 | + | Cl | Cl | Cl | Cl | H | —CH(CH₃)₂ |
| 17 | + | Cl | Cl | Cl | Cl | H | —CH₂-phenyl |
| 15 | + | Cl | Cl | Cl | Cl | H | -t-butyl |
| 13 | + | Cl | Cl | Cl | Cl | H | 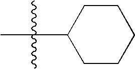 |
| 14 | + | Cl | Cl | Cl | Cl | H | -neopentyl |
| 809 | + | Cl | Cl | Cl | Cl | H | -i-propyl |
| 12 | + | Cl | Cl | Cl | Cl | H | 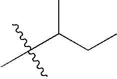 |
| 11 | + | Cl | Cl | Cl | Cl | H | 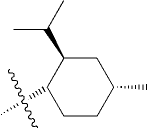 |
| 9 | + | Cl | Cl | Cl | Cl | H | 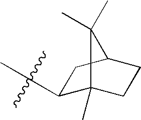 |

TABLE 4

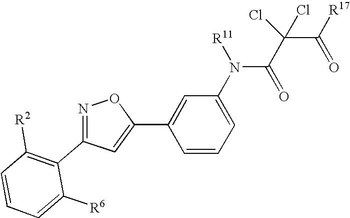

| Cpd | Replicon | R² | R⁶ | R¹¹ | R¹⁷ |
|---|---|---|---|---|---|
| 50 | + | Cl | Cl | H | 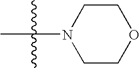 |
| 10 | − | Cl | Cl | H | -Me |

TABLE 5

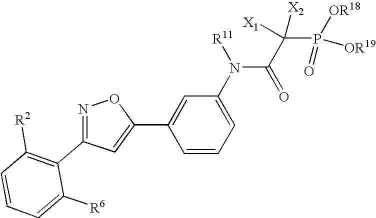

| Cpd | Replicon | X1 | X2 | R² | R⁶ | R¹¹ | R¹⁸/R¹⁹ |
|---|---|---|---|---|---|---|---|
| 405 | + | Cl | Cl | Cl | Cl | H | -Et/-Et |
| 407 | − | Cl | Cl | Cl | Cl | H | H/H |
| 5 | + | Cl | Cl | CF₃ | H | H | -Et/-Et |
| 6 | + | Cl | Cl | CF₃ | F | H | -Et/-Et |
| 4 | + | Cl | Cl | Cl | Cl | H | -i-propyl/-i-propyl |
| 2 | − | Cl | F | Cl | Cl | H | -EV-Et |
| 3 | − | F | F | Cl | Cl | H | -Et/-Et |

TABLE 6

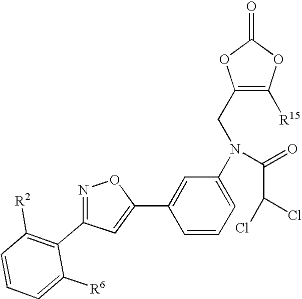

| Cpd | Replicon | R² | R⁶ | R¹⁵ |
|---|---|---|---|---|
| 901 | + | Cl | Cl | -Me |
| 31 | + | Cl | OMe | -Me |
| 34 | + | Cl | Cl | -i-propyl |
| 40 | + | Cl | Cl | -Et |
| 39 | + | Cl | Cl | 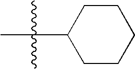 |
| 44 | − | Cl | Cl | C(O)OEt |
| 38 | + | Cl | Cl | -n-propyl |
| 36 | + | Cl | —C(O)O-t-butyl | -i-propyl |
| 37 | + | Cl | —OMe | -i-propyl |
| 1007 | + | Cl | Cl | -t-butyl |

TABLE 7

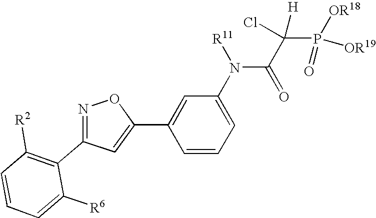

| Cpd | Replicon | R² | R⁶ | R¹¹ | R¹⁸/R¹⁹ |
|---|---|---|---|---|---|
| 1 | − | Cl | Cl | H | -Et/-Et |

TABLE 8

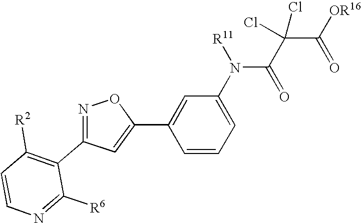

| Cpd | Replicon | R² | R⁶ | R¹¹ | R¹⁶ |
|---|---|---|---|---|---|
| 28 | + | Cl | —N(CH₃)₂ | H | -Me |
| 52 | − | 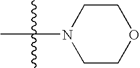 | Cl | H | 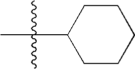 |
| 54 | − | 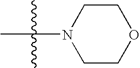 | Cl | H | -Et |
| 56 | − | 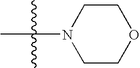 | Cl | H | -t-butyl |

TABLE 9

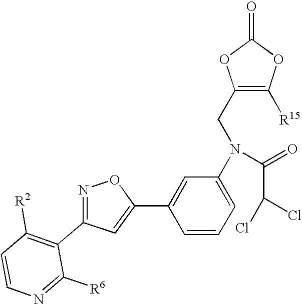

| Cpd | Replicon | R² | R⁶ | R¹⁵ |
|---|---|---|---|---|
| 32 | + | Cl | Cl | -Me |
| 33 | + | Cl | —N(CH₃)₂ | -Me |
| 35 | + | Cl | —N(CH₃)₂ | -i-propyl |

TABLE 10

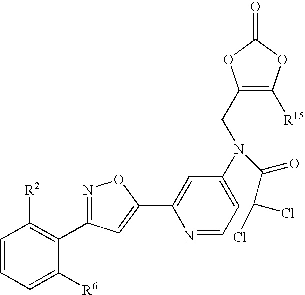

| Cpd | Replicon | R² | R⁶ | R¹⁵ |
|---|---|---|---|---|
| 41 | + | Cl | Cl | -Me |
| 42 | + | Cl | Cl | -i-propyl |
| 1107 | + | Cl | Cl | -t-butyl |
| 43 | + | Cl | Cl | -n-pentyl |

TABLE 11

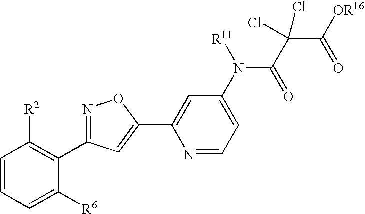

| Cpd | Replicon | R² | R⁶ | R¹¹ | R¹⁶ |
|---|---|---|---|---|---|
| 505 | + | Cl | Cl | H | -t-butyl |
| 23 | + | Cl | Cl | H | 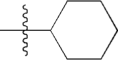 |

TABLE 12

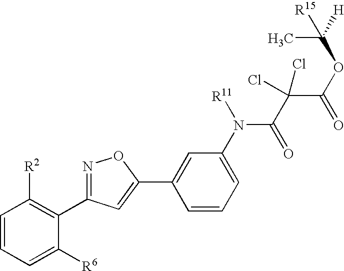

| Cpd | Replicon | R² | R⁶ | R¹¹ | R¹⁵ |
|---|---|---|---|---|---|
| 8 | + | Cl | Cl | H | C(O)OEt |

### Luciferase Counter Screen

A counter screen was used to identify non-specific inhibitors of the luciferase reporter gene. In the counter screen, a cell line carrying a construct such as a CMV-driven luciferase gene was used to identify metabolically active agents of the prodrugs that inhibit the reporter gene, and not HCV. In these CMV-Luc cells, the DNA construct, which comprises a luciferase gene downstream of a CMV promoter, is permanently integrated into the chromosome of Huh7 cells. For the counter screen, actively dividing CMV-Luc cells were seeded at a density of 5000-7500 cells/well in a volume of 90 ul/well into 96 well plate(s). The cells were then incubated at 37° C. and 5% CO₂ for 24 hours. Various concentrations of test prodrugs (in a volume of 10 ul) were added to the wells and the cells were incubated for 2448 hours. Media was aspirated from each well and Bright-Glo (Pharmacia) luciferase assay reagents were added to each well according to the manufacturer's manual. Luciferin counts were taken using a luminometer. IC₅₀ values were greater than 10 pM in the counter screen luciferase inhibition assay for the metabolically active agents of the prodrugs of TABLES 1 through 12 that were tested.

### PCR Assay

A TaqMan RT-PCR assay (Roche Molecular Systems, Pleasanton, CA) was used to analyze HCV RNA copy numbers, which confirmed that the viral genome of HCV is not being replicated. Actively dividing 9-13 replicon cells were seeded at a density of 3×10⁴ cells/well in a volume of 1 ml/well into 24-well plates. The cells were then incubated at 37° C. and 5% CO₂ for 24 hours. Various concentrations of test prodrugs (in a volume of 10 ul) were added to the wells and the cells were incubated for an additional 24-48 hours. Media was removed by aspiration and RNA samples prepared from each well. TaqMan one step RT-PCR (Roche Molecular Systems, Alameda, CA) was performed using the freshly prepared RNA samples according to the manufacturer's manual and analyzed on an ABI Prism 7700 Sequence Detector (Applied Biosystems). The ratio of HCV RNA to cellular GAPDH RNA was used as in indication of specificity of HCV inhibition to confirm that the viral genome was not replicated.

### HCV Infection Assay

The activity of a prodrug that is metabolized into an active agent can also be confirmed in an HCV infection assay. The assay can be carried out essentially as described in Fournier et al., 1998, J. Gen. Virol. 79:2367-2374. Briefly, hepatocyte cells from a doner can be plated on Day 1. On Day 3, the cells would be inoculated with HCV virus and test prodrug added. On Day 5, the medium would be changed and test prodrug would be added. On Day 7, the medium would be changed and test prodrug would be added. On Day 8, the RNA would be isolated and the HCV RNA quantified using a Taqman assay. Prodrugs that are metabolized into an active agent that exhibit an IC₅₀ of less than 10 μM in this assay can be identified.

### Determination of Non-Toxicity of Prodrugs in Cell Culture

Prodrugs can be tested in a cytotoxicity assay with liver cells including an HCV replicon (5-2 Luc cells, 9-13 cells or Huh-7 cells). In the assay, cells can be seeded onto 96-well plates (approx. 7500 cells/well in a volume of 90 μl) and grown for 24 hr at 37° C. On day 2, various concentrations of test prodrug (in a volume of 10 82 l) would be added to the wells and the cells would be grown for an additional 48 hr at 37° C. On day 3, an ATP-dependent R-Luciferase assay (Cell Titer Glo assay) would be performed to determine the number of viable cells. Prodrugs that are metabolized into an active agent exhibiting an CC₅₀ of greater than 10 μM would be considered as non-toxic.

### Animal Studies

The safety of prodrugs can be evaluated in rats by oral, subcutaneous and intravenous administration in several experiments. Doses as high as 30 mg/kg/day can be monitored. Experimental procedures are summarized below.

In a first study the toxicity of prodrugs can be evaluated either by the subcutaneous (SC) route or the intravenous (IV via jugular cannula) route of administration in Sprague Dawley rats. Two male rats would be used in each group. A dose escalation scheme would be employed where a prodrug would be delivered IV or SC for 3 consecutive days at a dose of 10 mg/kg (study Days 1-3) in a 80%:20% - PEG/water vehicle; delivered one day IV or SC dose of 30 mg/kg (study Day 4) in 100% PEG; and an IV dose of 60 mg/kg (study Day 5) in 100% PEG. Prodrugs could be identified as being well tolerated at doses up to and including 30 mg/kg by both routes of administration.

In a second study prodrugs can be was administered by the IV route at doses of 10 and 30 mg/kg in 100% PEG. The volume administered for the 10 mg/kg dose would be 0.67 ml/kg/day and volume given the 30 mg/kg group would be 2 ml/kg/day. In addition, there would be two control groups. One control would receive 100 % PEG alone at a volume of 2 ml/kg/day while the other would be an untreated sham control group. All groups (except for the untreated control with 3 male rats) would include 4 male rats each. Parameters of study would include: clinical observations, body weights, hematology, clinical chemistry, gross necropsy, organ weights, bone marrow assessment and histopathology of selected organs. Decreases in red blood cells, hemoglobin and hematocrit relative to the untreated control but not the vehicle control could be determined.

In a third study prodrugs can be compared with other compounds and administered at a dose of 10 and 30 mg/kg in 100 % PEG and delivered by IV at a concentration of 1 mI/kg/day first via a jugular cannula and when the cannula failed by the lateral tail vein. A vehicle control group would receive the 100% PEG alone at the same volume. Groups would comprise 3 males and 3 females each. Before reducing the dose to 10 and 30 mg/kg two rats would receive 100 mg/kg IV at a volume of 1 ml/kg. Parameters of study would include: clinical observations, body weights, hematology, clinical chemistry, gross necropsy, organ weights and histopathology of selected organs (including injection sites).

### Sustained Plasma Levels

The pharmacokinetic properties of prodrugs can be calculated in rats, monkeys and chimpanzees using the intravenous and subcutaneous routes of administration with a variety of different delivery vehicles. Sustained plasma levels can be determined with several different liposome suspension vehicles using subcutaneous administration: (i) 5 mg/ml prodrug in water with 100 mg/ml lecithin; (ii) 5 mg/ml prodrug in water with 200 mg/ml lecithin; and (iii) 5 mg/ml prodrug in water with 100 mg/ml lecithin and 5 mg/ml cholesterol. Based on these results, it is expected that other liposome formulations as are well-known in the art may be used to administer the prodrugs of the invention

The prodrugs of the invention are rapidly metabolized in microsomes from rat and human livers and, in part, converted to the active compound, A-B-C-NHCOCHX₂. Since the active compounds are degraded rapidly by non-NADPH dependent esterases, an esterase inhibitor, bis(p-nitrophenyl) phosphate (BNPP), was used in microsomal incubations to prevent degradation of the active compound. BNPP has been shown, with a number of prodrugs, to have no effect on the disappearance rate of the prodrugs in either rat or human microsomes. Metabolism of the prodrugs occurs by NADPH dependent enzyme(s), presumably P450. Although all of the prodrugs are rapidly metabolized in liver microsomes, the amount of active compound produced varies significantly among the compounds tested. Metabolic pathways have not been extensively explored but, for many of the compounds, the absence of the deacetylation product has been confirmed using LC/MS/MS. That is, the prodrug side-chain serves to prevent esterase attack leading to inactive, deacetylation products. Loss of the alkyl side-chain seems to be the preferred metabolic pathway. Additional studies have been conducted in microsomes isolated from human jejunum. The compounds, in general, are stable in these gut microsomes.

### Methods

Incubations were conducted using cryopreserved rat and human microsomes purchased from commercial sources. Incubations were conducted at a final protein concentration of lmg/ml in phosphate buffer (100 mM, pH=7.4) containing 1 mM NADPH. BNPP concentrations were 1 mM in the incubation mixture. Reaction was started by addition of the compounds (5 μl of 10 μg/ml in water:DMSO; 9:1). Reactions were conducted at a volume of 100 μL in 96-well plates and reactions were quenched by addition of 50 μl of dimethyl sulfoxide containing 1% formic acid. Samples were prepared for analysis by addition of an internal standard (verapamil, 10 μl of 0.5 μM in water) and 100 μl of an organic solvent mixture (Acetonitrile:ethanol:DMSO; 2:1:1). Samples were centrifuged before analysis to remove precipitated proteins.

### LC/MS/MS Analysis

A PE/Sciex 3000 instrument was used for all analyses. Samples were injected onto a Betasil C8 column (50×3mm; Thermo Electron Corp.) and eluted with an acetonitrile gradient. A heated nebulizer source was used as the interface between the HPLC column and the MS/MS. Quantitative analyses were conducted using the MRM mode of operation with specific parameters determined, in advance, for each compound. Standards were prepared in rat or human microsomes using the procedures outlined above and covered a range from 1-1000 ng/ml.

### Pharmokinetic Studies

Pharamcokinetic studies were conducted in rats containing surgically implanted cannula in the portal and jugular veins. Blood samples were taken simultaneously from both cannula at various times after oral administration of the compounds. In the portal vein, the prodrugs are detected and, in some cases, the active compound, A-B-C-NHCOCHX₂, has also been detected. The presence of active compound in the portal vein samples has been attributed to metabolism of the prodrug in transit through the gut wall. Surprisingly, the levels of inactive metabolite have been low. In the jugular samples, many, but not all, of the prodrugs have been detected in systemic circulation, depending on the hepatic extraction ratio of the prodrug. In contrast, oral administration of A-B-C-NHCOCHX₂ (where the “C” ring is a 2-pyridyl) results in high levels of inactive metabolite in the portal vein (attributed to esterase activity in the small intestine) and low levels of A-B-C-NHCOCHX₂.

Evidence for the conversion of prodrug 1107 to its active metabolite in rat liver was obtained by measuring prodrug and active compound concentrations in bile fluid. High levels of 1107 and resultant A-BC-NHCOCHX₂ were detected in bile fluid for a period of four hours after oral administration. In contrast, no A-B-C-NHCOCHX₂ was detected in bile fluid after oral administration of equivalent A-B-C-NHCOCHX₂. The data suggests that prodrug 1107 accumulates in the liver and is slowly converted to the active dihaloacetamide. See Table 13.

### Methods

Compounds were dissolved in a mixture of TPGS:PEG:PG (35%:60%:5%) and diluted with saline for oral administration. Typically, 6.5mg of compound was dissolved in 1 ml of the organic mixture and added to 5.5 ml of saline. Animals were dosed with 5 ml/kg of this mixture to give a typical dose of 5 mg/kg. All animal studies were conducted in male SpraqueDawley rats and the animals were fasted overnight prior to dose administration. Surgeries (to implant blood sampling cannula) were conducted at least two days prior to the study. Blood samples (100 μl) were collected using sodium heparin as anticoagulant and added to 300 μl of a mixture of acetate buffer (100 mM, pH=6.5):acetonitrile:ethanol:DMSO (2:1:1:2 v/v). Samples were centrifuged and the supernatant was analyzed by LC/MS/MS using the procedures outlined above. Standard were prepared similarly using fresh rat blood from naive animals. See Table 13.

TABLE 13

| | | Microsomal Stability | | | | Rat PK | | |
|---|---|---|---|---|---|---|---|---|
| | | | | % Conversion | | | | Hepatic |
| | | Half-life (min.) | | to active agent | | Cmax, ng/ml | | extraction |
| | | | | | | | Active | ratio of |
| Cpd | Structure | Rat | Human | Rat | Human | Prodrug | Agent | Prodrug |
| 1107 | 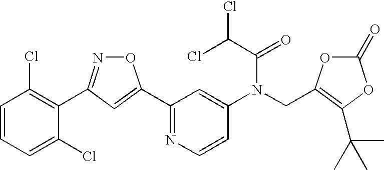 | — | — | — | ˜50 | 510 | 50 | 0.97 |

TABLE 14

| Cpd. No. | Assay | Result Value (μM) |
|---|---|---|
| 1018 | HEPC_REPLICON-ICWESTERN | +++ |
| 1021 | HEPC_REPLICON-ICWESTERN | ++ |
| 1022 | HEPC_REPLICON-ICWESTERN | +++ |
| 1023 | HEPC_REPLICON-ICWESTERN | +++ |
| 1024 | HEPC_REPLICON-ICWESTERN | +++ |
| 1026 | HEPC_REPLICON-ICWESTERN | + |
| 1027 | HEPC_REPLICON-ICWESTERN | ++ |
| 1028 | HEPC_REPLICON-ICWESTERN | + |
| 1037 | HEPC_REPLICON-ICWESTERN | + |
| 1038 | HEPC_REPLICON-ICWESTERN | ++ |
| 1039 | HEPC_REPLICON-ICWESTERN | + |
| 1040 | HEPC_REPLICON-ICWESTERN | ++ |
| 1041 | HEPC_REPLICON-ICWESTERN | ++ |
| 1041 | HEPC_REPLICON-LUC | ++ |
| 1042 | HEPC_REPLICON-ICWESTERN | + |
| 1043 | HEPC_REPLICON-ICWESTERN | ++ |
| 1043 | HEPC_REPLICON-LUC | ++ |
| 1044 | HEPC_REPLICON-ICWESTERN | +++ |
| 1044 | HEPC_REPLICON-LUC | +++ |

The results of the Replicon assays are provided in Tables 14 and 15. In Table 14, a value of “+++” means less than 1 μM; ++ means between 1 and 20 μM; + means greater than 20 μM. In TABLE 15, a value of “+” indicates an IC₅₀ of 10 μM or less in the specified assay; a value of “−” indicates an C₅₀ of greater than 10 μM in the specified assay.

TABLE 15

| Cpd No. | Replicon |
|---|---|
| 100a | + |
| 102a | + |
| 103a | + |
| 104a | + |
| 105a | + |
| 106a | + |
| 107a | + |
| 108a | + |
| 109a | + |
| 110a | + |
| 111a | + |
| 112a | + |
| 113a | + |
| 114a | + |
| 115a | + |
| 116a | + |
| 117a | + |
| 118a | + |
| 119a | + |
| 120a | + |
| 121a | − |
| 122a | − |
| 123a | + |
| 124a | + |
| 125a | − |
| 126a | + |
| 127a | + |
| 128a | + |
| 129a | + |
| 130a | + |
| 131a | + |
| 132a | + |
| 133a | + |
| 134a | + |
| 135a | + |
| 136a | + |
| 137a | + |

### Stabilization of the Active Parent Compound by a Prodrug Approach

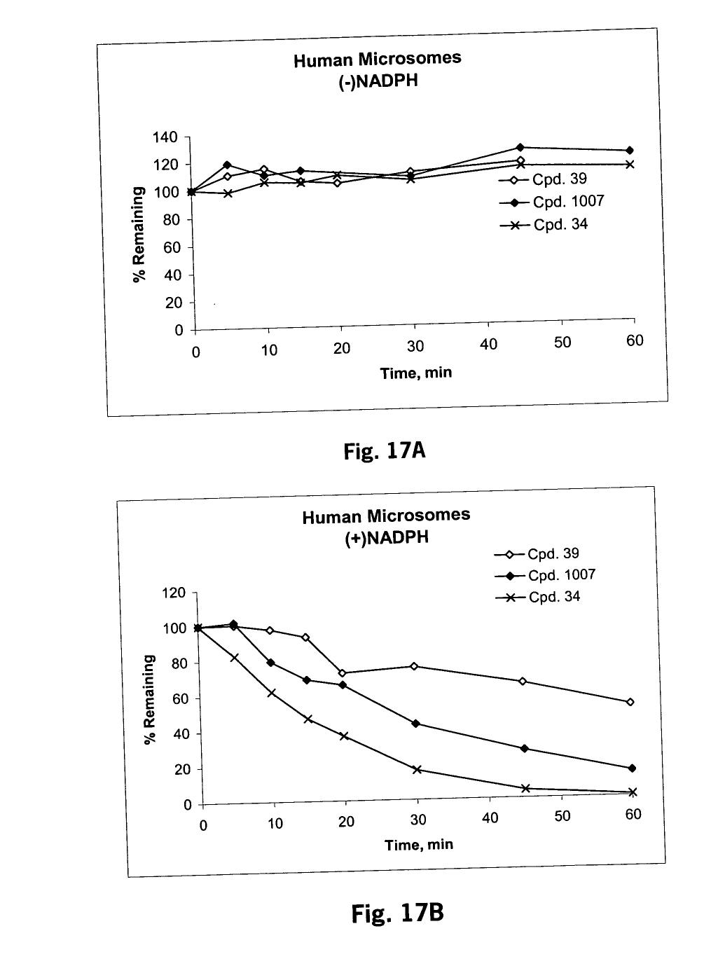

The active parent compound is cleaved by esterase enzymes to an inactive metabolite in a reaction (for example, see scheme below) that does not require NADPH as a cofactor. Alkylation of the acetyl nitrogen stabilizes the active parent compound against direct attack by esterases and yet, if NADPH is added as a cofactor, conversion to the active parent compound occurs through CYP P450 enzyme activity. Because esterases are present in the gut, the active parent compound is extensively hydrolyzed during the absorption process. The prodrug approach prevents gut hydrolysis of the active parent compound and, because the liver contains high levels of P450 activity, the active parent compound is generated within the liver. Some results are shown in FIGS. 17A and 17B.

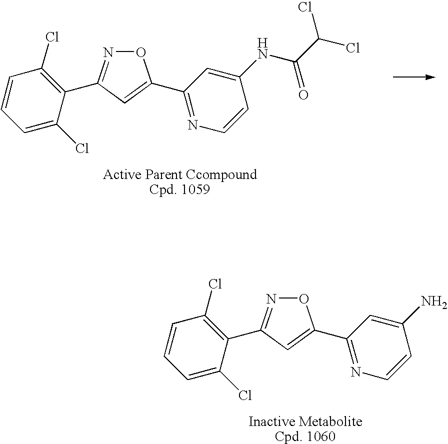

### Oral Administration of the Active Parent Compound—Hydrolysis in Rats

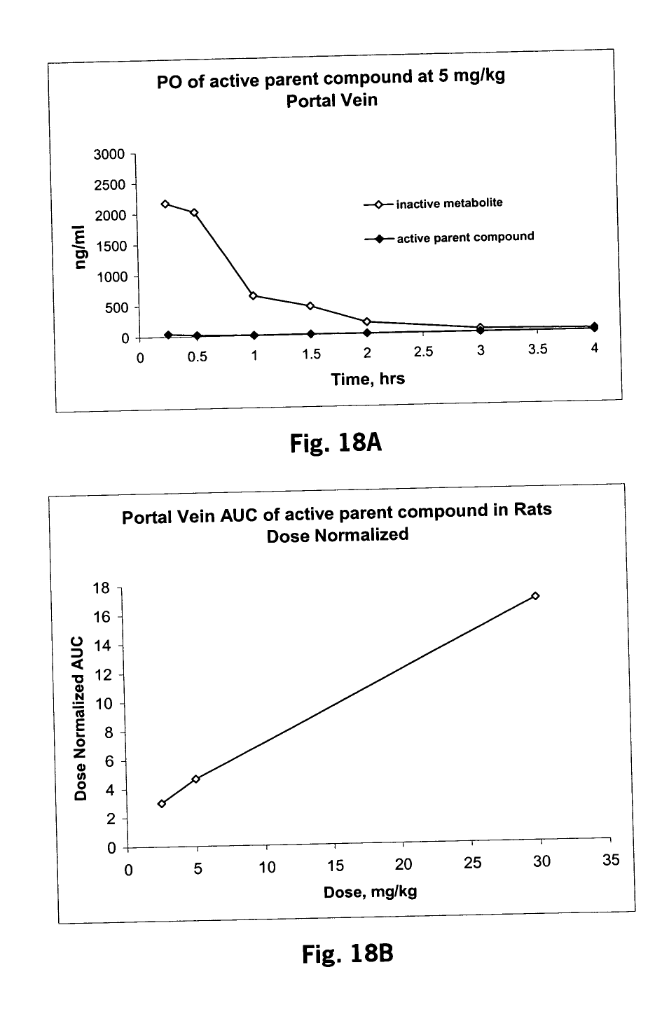

Administered orally, the active parent compound is well absorbed but is extensively hydrolyzed to the inactive metabolite as demonstrated in this experiment in which plasma samples were taken from the portal vein of rats and analyzed for the active parent compound and the inactive metabolite. However, a portion of the active parent compound remains intact and the active parent compound concentrations increase in a linear fashion with dose. Because the portal blood flows directly to the liver, this intact the active parent compound may exert an anti-viral effect in the liver. Some results are shown in FIGS. 18A and 18B.

| Dose | Ratio Cpd. 1060/ |
|---|---|
| (mg/kg) | Cpd. 1059 |
| 2.5 | 29 |
| 5 | 88 |
| 30 | 24 |

### Hydrolysis of the Active Parent Compound in the Cynomolgus Monkey

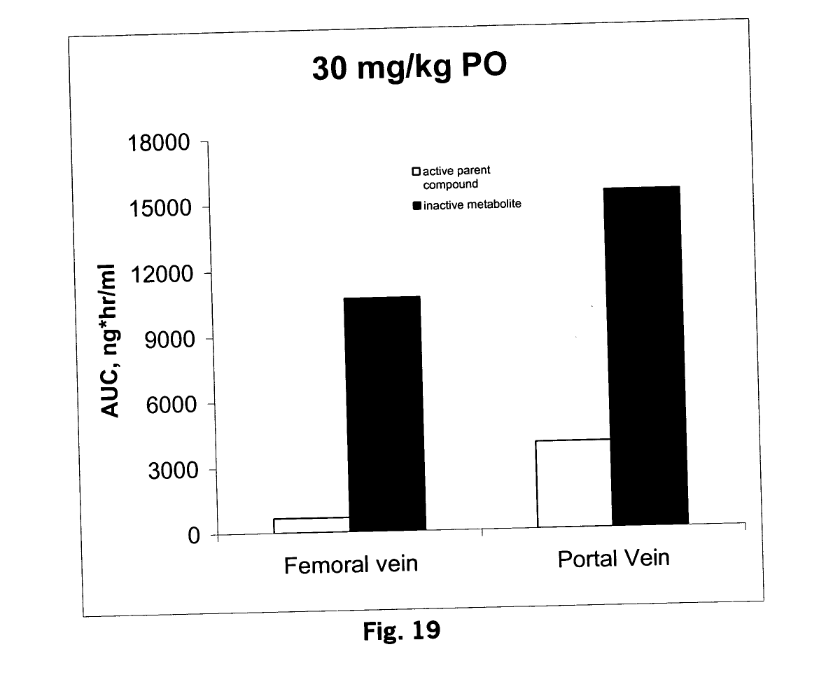

Consistent with results in rats, the active parent compound is extensively hydrolyzed in the gut of cynomolgus monkeys as shown by the high ratio of the inactive metabolite to the active parent compound in the portal vein of monkeys. A portion of the active parent compound present in the portal vein (and the inactive metabolite as well) is extracted by the liver resulting in lower concentrations reaching systemic circulation. The results are shown in FIG. 19.

### Biliary Excretion of the Active Parent Compound and the Inactive Metabolite in Rats Following Oral Administration of the Active Parent Compound

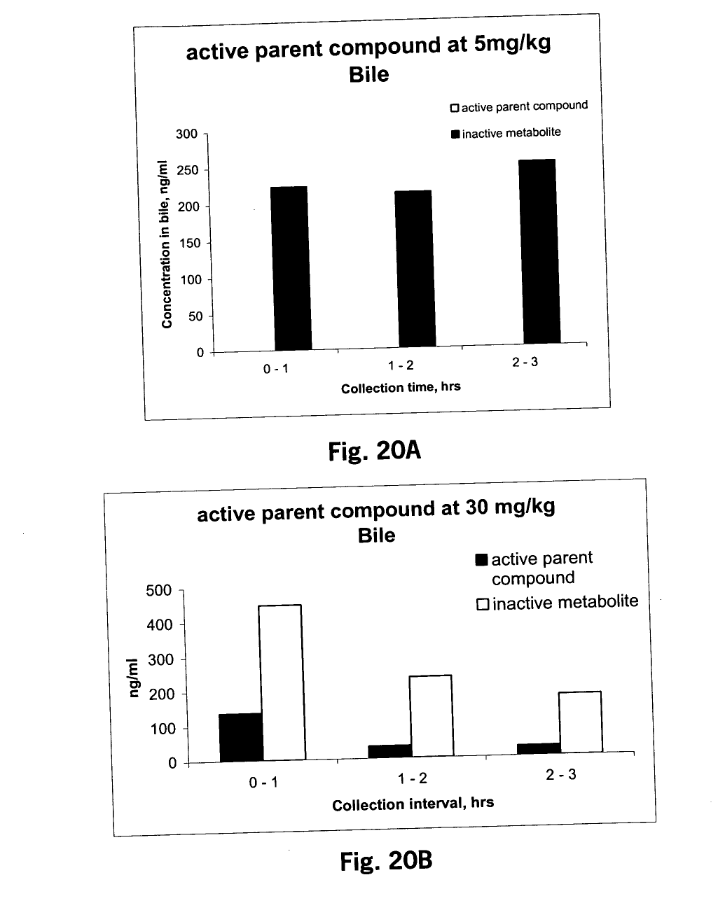

The active parent compound that remains intact during absorption through the gut wall is primarily extracted in the liver and does not reach systemic circulation. Esterases are present in various tissues in the liver and may further degrade the active parent compound prior to reaching the HepC virus that is localized within hepatocyte cells. Experiments with radio-labeled the active parent compound indicate that the active parent compound is excreted from the body through bile (data not shown). In the experiment shown below, two doses of the active parent compound were administered orally to rats. Bile was collected in one-hour intervals for three hours. At the lower dose, 5 mg/kg, no intact the active parent compound was detected in the bile. At the higher dose, 30 mg/kg, the active parent compound was detected in bile—suggesting that the active parent compound escaped esterase activity. At both doses, however, the inactive metabolite concentrations in bile were substantially higher than the active parent compound concentrations. As discussed above, the inactive metabolite is generated, in part, in the gut. Additional inactive metabolite may be generated in the liver itself. The goal of the prodrug strategy has been to reduce or eliminate exogenous metabolism of the active parent compound and to generate the active parent compound in-situ (i.e. within hepatocytes) using enzymes which are endogenous to hepatocytes. The results are shown in FIGS. 20A and 20B.

### Absorption of the Active Parent Compound and Prodrugs in Cynomolgus Monkeys

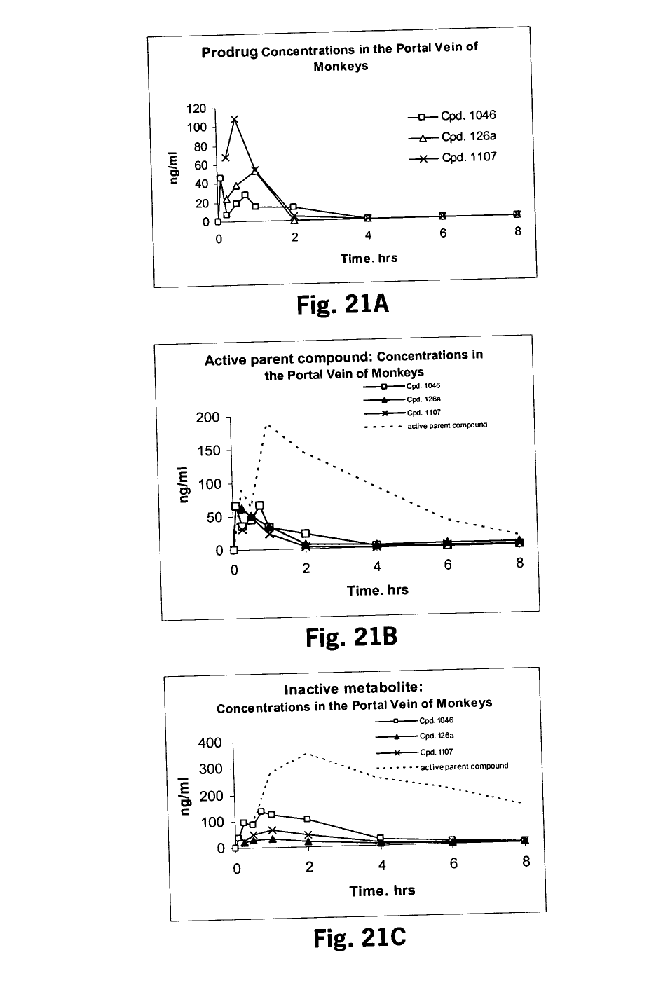

A number of prodrugs of the active parent compound, and the active parent compound itself, were administered orally to cynomolgus monkeys and portal vein samples were analyzed. All of the prodrugs administered were detected in the portal vein and partial conversion of the prodrugs to the active parent compound was observed (thought to be the result of low levels of P450 activity present in the gut epithelium). The levels of the inactive metabolite were lower than those generated from direct administration of the active parent compound—the prodrug approach prevents gut metabolism to inactive inactive metabolite. The results are shown in FIGS. 21A, 21B and 21C.

### Biliary Excretion of the Active Parent Compound and the Inactive Metabolite Following Oral Administration of Prodrugs to Rats

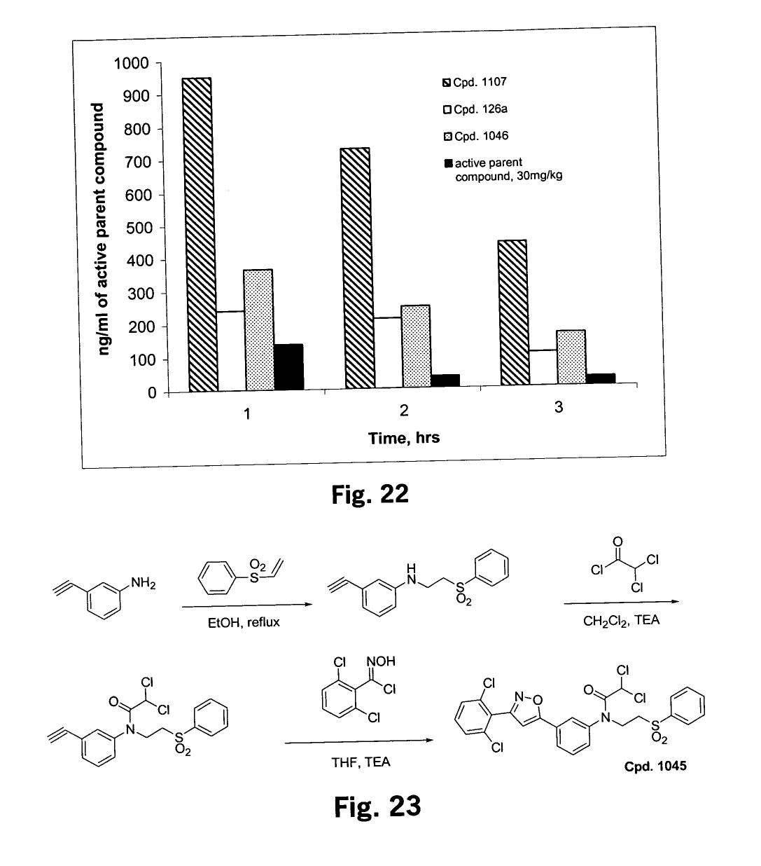

Prodrugs were administered at a dose of 5mg/kg, the active parent compound was administered at a dose of 30mg/kg. Bile was collected for three hours in one-hour intervals and analyzed for the active parent compound. The data indicates that the prodrugs reached the liver, were converted into the active parent compound and that the the active parent compound was excreted unchanged. The concentrations of the active parent compound in the bile were substantially higher for a 5mg/kg dose of prodrug than for a 30mg/kg dose of the active parent compound. The results are shown in FIG. 22.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. AMENDMENTS

1. A compound according to structural formula
A—B—C−(C(O))ₛ—N(R¹¹)—C(O)—CX₂—R¹²
or a pharmaceutically acceptable salt, hydrate, solvate or N-oxide thereof, wherein
A is a phenyl or six-membered heteroaryl ring having from one to five of the same or different R²⁰ substituents, provided that at least one of the substituents is positioned at the ortho position;
B is a saturated, unsaturated, or aromatic heteroatomic ring having from one to three annular heteroatoms selected from N, O, and S, where the A and C moieties are attached to non-adjacent ring atoms of B, provided that when the B includes more than one annular oxygen atom, the oxygen atoms are not adjacent;
C is a phenyl or a heteroaryl ring, wherein when C is phenyl, it is substituted relative to the B moiety at the meta position with the —(C(O))₁—N(R¹¹)—C(O)—CX₂—R¹², or when C is a heteroaryl group, the B moiety and the -(C(O))₁—N(R¹¹)—C(O)—CX₂—R¹² moiety are positioned on C with only one ring atom of C between them;
s is 0 or 1;
R¹¹ is selected from the group consisting of hydrogen, lower alkyl, -(CHR¹⁰)ₙ—J—G, or a group of the formula 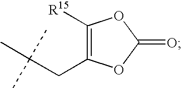
each X is independently H or halo, provided both X are not H;
R¹² is selected from the group consisting of hydrogen, —O—C(O)-alkyl, —C(O)OR¹⁶, —C(O)R¹⁷ and —P(O)(OR¹⁸)OR¹⁹;
R¹⁵ is lower alkyl, arylalkyl, aryl, substituted cycloheteroalkyl, cycloheteroalkyl, substituted cycloalkyl, cycloalkyl, —C(O)OR¹⁸ or —CH₂—OR³⁰;
R³⁰ is hydrogen, lower alkyl or a sugar moiety;
R¹⁶ is selected from the group consisting of aryl-C₁-C₆ alkyl, aryl, substituted cycloheteroalkyl, cycloheteroalkyl, substituted cycloalkyl, cycloalkyl, —C(O)OR¹⁶ or —CH₂—OR³⁰, (C₁₋₁₅) alkyl and (C₇-₁₅) arylalkyl;
R¹⁷ is selected from the group consisting of lower alkyl, —N(R^{c})₂, N-morpholino, N-piperazino and N-pyrrolidino;
each R^{c} is independently R^{b} or alternatively, the both R^{c} taken together with the nitrogen atom to which they are bonded form a 5-, 6- or 7-membered cycloheteroalkyl which optionally includes from 1 to 4 of the same or different additional heteroatoms selected from the group consisting of O, N and S;
each R^{b} is independently hydrogen or R^{a};
R^{a} is selected from the group consisting of alkyl, hydroxyalkyl, cycloalkyl, heteroalkyl, C₀-C₆ alkyl-cycloheteroalkyl, substituted C₀-C₆ alkyl-cycloheteroalkyl, aryl, arylalkyl, heteroaryl and heteroarylalkyl;
R¹⁸ is H, lower alkyl, aryl or arylalkyl;
R¹⁹ is H, lower alkyl, aryl or arylalkyl;
n is 0, 1, 2, 3 or 4;
each R¹⁰ is independently hydrogen or lower alkyl;
J is selected from the group consisting of -(CH₂)₁₋₃-, —O—, —O—(CH₂)₁₋₃-, —CH(OH)-, −C(═O)-, —S(O)ₘ-, —C(═NR²³)-, —C(═NOR²⁹)-, —C(N—N—(R²⁵)₂)-, —C(N−NR²⁷C(═O)N—(R²⁷)₂)- and —C(Z—R²⁸)₂-;
m is 0, 1, or 2;
R²³ is selected from the group consisting of hydrogen, lower alkyl, aryl, substituted aryl, arylalkyl and substituted arylalkyl;
R²⁹ is selected from the group consisting of hydrogen, lower alkyl, aryl, substituted aryl, arylalkyl and substituted arylalkyl;
each R²⁵ is independently selected from the group consisting of hydrogen, lower alkyl, aryl, substituted aryl, arylalkyl and substituted arylalkyl;
each R²⁷ is independently selected from the group consisting of hydrogen, lower alkyl, aryl, substituted aryl, arylalkyl and substituted arylalkyl;
each Z is independently —O— or —S—;
each R²⁸ is independently selected from the group consisting of lower alkyl, aryl, substituted aryl, arylalkyl and substituted arylalkyl or alternatively, the two R¹⁸'s taken together with the heteroatoms to which they are bonded form a 5, 6 or 7 membered cycloheteroalkyl;
G is selected from the group consisting of aryl, substituted aryl, arylalkyl, substituted arylalkyl, heteroaryl, substituted heteroaryl, cycloheteroalkyl and substituted cycloheteroalkyl; and
each R²⁰ is, independently of the other, selected from the group consisting of —OH, —SH, —CN, —C(O)H, —NO₂, halo, fluoro, chloro, bromo, iodo, lower alkyl, substituted lower alkyl, lower heteroalkyl, substituted lower heteroalkyl, cycloalkyl, substituted cycloalkyl, cycloheteroalkyl, substituted cycloheteroalkyl, lower haloalkyl, monohalomethyl, dihalomethyl, trihalomethyl, trifluoromethyl, lower alkylthio, substituted lower alkylthio, lower alkoxy, substituted lower alkoxy, methoxy, substituted methoxy, lower heteroalkoxy, substituted lower heteroalkoxy, cycloalkoxy, substituted cycloalkoxy, cycloheteroalkoxy, substituted cycloheteroalkoxy, lower haloalkoxy, monohalomethoxy, dihalomethoxy, trihalomethoxy, trifluoromethoxy, amino, lower di- or monoalkylamino, substituted lower di- or monoalkylamino, aryl, substituted aryl, aryloxy, substituted aryloxy, phenoxy, substituted phenoxy, arylalkyl, substituted arylalkyl, arylalkyloxy, substituted arylalkyloxy, benzyl, benzyloxy, heteroaryl, substituted heteroaryl, heteroaryloxy, substituted heteroaryloxy, heteroarylalkyl, substituted heteroarylalkyl, heteroarylalkyloxy, substituted heteroarylalkyloxy, carboxyl, lower alkoxycarbonyl, substituted lower alkoxycarbonyl, aryloxycarbonyl, substituted aryloxycarbonyl, arylalkyloxycarbonyl, substituted arylalkyloxycarbonyl, carbamate, substituted carbamate, carbamoyl, substituted carbamoyl, thiocarbamoyl, substituted thiocarbamoyl, ureas, substituted ureas, thioureas, substituted thioureas, sulfamoyl, substituted sulfamoyl and a group of the formula —L—R¹⁴, where “L” is a linker and R¹⁴ is cycloalkyl, substituted cycloalkyl, cycloheteroalkyl or substituted cycloheteroalkyl;
provided that when R¹¹ is hydrogen or lower alkyl, then R¹² is not hydrogen.

2. The compound according to claim 1, wherein s is 1, R¹² is —H, and R¹¹ is a group of the formula 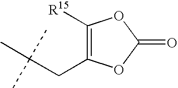
wherein R¹⁵ is —CH₂—OR³⁰.

3. The compound according to claim 2, wherein R³⁰ is a sugar moiety.

4. The compound according to claim 1, wherein s is 0.

5. The compound according to claim 4, wherein R¹² is —H, R¹¹ is a group of the formula 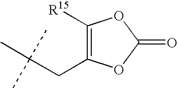
wherein R¹⁵ is lower alkyl, arylalkyl, aryl, cycloheteroalkyl, cycloalkyl, or —CH₂—OR³⁰.

6. The compound according to claim 5, wherein R¹⁵ is piperidyl, pyrrolidinyl, t-butyl, benzyl, cyclobutyl or propyl.

7. The compound according to claim 6, wherein R¹⁵ is —CH₂—OR³⁰.

8. The compound according to claim 7, wherein R³⁰ is a sugar moiety.

9. The compound according to claim 1, wherein s is 0 and R¹¹ is selected from the group consisting of hydrogen or -(CHR¹⁰)ₙ—J—G, wherein n is 0, 1 or 2, J is -(CH₂)₁₋₃-, —C(O)—, —O— or —O—(CH₂)₁₋₃-, and G is substituted aryl, cycloheteroalkyl, substituted cycloheteroalkyl or heteroaryl.

10. The compound according to claim 9, wherein R¹¹ is hydrogen.

11. The compound according to claim 9, wherein G is pyrrolidinyl, morpholinyl or imidazolyl.

12. The compound according to claim 9, wherein G is phenyl substituted with methoxy, -chloro, fluoro, CH₂—P(O)(OR^{b})(OR^{b}), —O—P(O)(OR^{b})(OR^{b}), methyl, —O—C(O)—NH—R^{b}, —NR^{b}C(O)OR^{b}, ethyl-piperazinyl, piperazinyl, t-butyl-O-C-(O)-piperazinyl, —O—(CH₂)₀₋₄—R^{b}, or —C(O)OR^{b}, wherein R^{b} is —H, propyl, t-butyl, ethyl, or morpholinyl.

13. The compound according to claim 1, wherein
s is 0;
each X is chloro, and R¹² is selected from the group consisting of hydrogen, —C(O)OR¹⁶, —C(O)R¹⁷ and —P(O)(OR¹⁸)OR¹⁹;
R¹⁶ is lower alkyl, arylalkyl, substituted cycloalkyl, or cycloalkyl;
R¹⁷ is selected from the group consisting of lower alkyl, —N(R^{c})₂, or N-morpholino;
each R^{c} is independently hydrogen, alkyl, hydroxyalkyl, C₀-C₆ alkyl-cycloheteroalkyl, or heteroarylalkyl; and
R¹⁸ and R¹⁹ are independently H or lower alkyl.

14. The compound according to claim 13, wherein R¹² is —P(O)(OR¹⁸)OR¹⁹.

15. The compound according to claim 14, wherein R¹⁸ and R¹⁹ are both —H, ethyl or propyl.

16. The compound according to claim 13, wherein R¹² is —C(O)OR¹⁶.

17. The compound according to claim 16, wherein R¹⁶ is adamantane methyl, propyl, —CH₂-phenyl, t-butyl, cyclohexyl, or cyclohexyl substituted with methyl, propyl, or pentyl.

18. The compound according to claim 13, wherein R¹² is —C(O)R¹⁷.

19. The compound according to claim 18, wherein R¹⁷ is methyl, N-morpholino, or —N(R^{c})₂.

20. The compound according to claim 19, wherein each Rc is independently hydrogen, -(CH₂)₃-morpholinyl, —CH₂-pyridinyl or -(CH₂)₂—OH.

21. The compound of claim 1, wherein s is 0 and A is phenyl substituted with at least two R²⁰ groups selected from the group consisting of halo, lower alkoxy, carboxyl, lower haloalkyl, cycloalkyl, lower alkoxycarbonyl and —L—R¹⁴, wherein “L” is a —O— and R¹⁴ is cycloheteroalkyl or substituted cycloheteroalkyl.

22. The compound of claim 21, wherein A represents a phenyl ring substituted at the 2- and 6-positions with the same or different R²⁰ substituent.

23. The compound of claim 22, wherein one R²⁰ is halo and the other R²⁰ is lower alkoxy, lower haloalkyl or cycloalkyl.

24. The compound of claim 23 wherein one R²⁰ is chloro and the other R²⁰ is methoxy, —CF₃, or cyclopropyl.

25. The compound of claim 22, wherein one R²⁰ is halo and the other R²⁰ is carboxyl or lower alkoxycarbonyl.

26. The compound of claim 25, wherein one R²⁰ is chloro and the other R²⁰ is methoxycarbonyl.

27. The compound of claim 22 wherein one R²⁰ is halo and the other R²⁰ is —O—R¹⁴, wherein R¹⁴ is morpholinyl or morpholinyl substituted with —C(O)—O—t-butyl or —C(O)—CH₃.

28. The compound of claim 1, wherein s is 0 and A represents a pyrid-2-yl ring substituted at the 3-position with an R²⁰ substituent, a pyrid-3-yl ring substituted at the 2- and 4-positions with the same or different R²⁰ substituents or a pyrid-4-yl ring substituted at the 3- and 5-positions with the same or different R²⁰ substituents.

29. The compound according to claim 28, wherein each R²⁰ is independently selected from the group consisting of halo, lower dialkylamino and —L—R¹⁴, wherein “L” is a —O— and R¹⁴ is cycloheteroalkyl.

30. The compound according to claim 29, wherein each R²⁰ is independently selected from the group consisting of chloro, —N(CH₃)₂, and —O—R¹⁴, wherein R14 is morpholinyl.

31. The compound according to claim 1, wherein s is 0 and C represents a phenyl ring, a pyrid-2-yl ring or a pyrid-3-yl ring.

32. The compound of claim 1, wherein s is 0 and B represents a isoxazolyl, pyrazolyl, oxadiazolyl or triazolyl ring.

33. The compound of claim 1, wherein s is 0 and B is 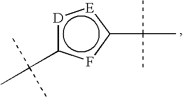
where D, E and F are each, independently of one another, selected from N, O and CH, provided that at least two of D, E and F are other than CH and D and E are not both simultaneously O.

34. The compound of claim 1, wherein s is 0 and each X is —Cl.

35. The compound of claim 34, wherein is selected from the group consisting of: 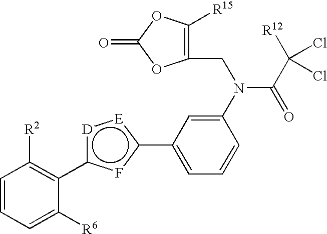 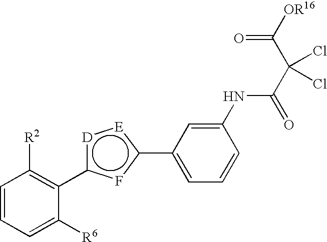 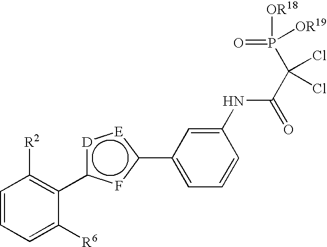 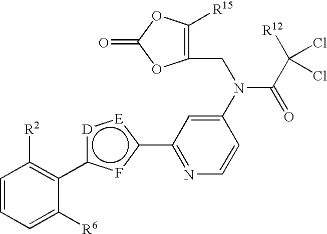 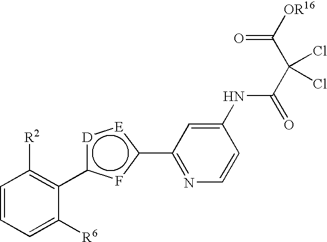 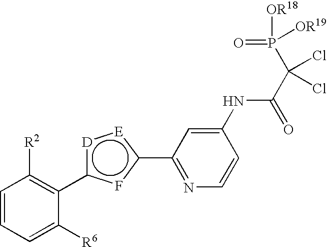 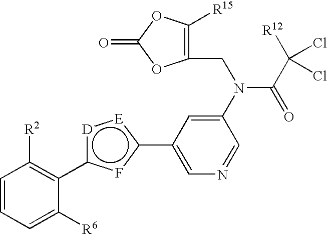 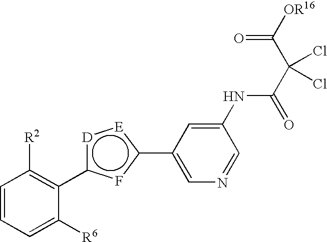 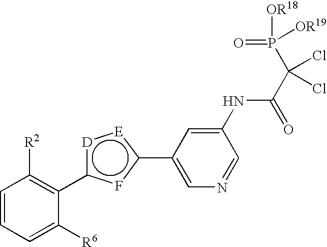
wherein R² and R⁶ are R²⁰ and each, independently of one another, is selected from the group consisting of —OH, —NO₂, halo, fluoro, chloro, bromo, iodo, lower alkyl, methyl, lower heteroalkyl, (C₃-C₆) cycloalkyl, 5- or 6-membered cycloheteroalkyl, N-morpholinyl, N-methyl-N-piperazinyl, N-piperadinyl, substituted N-piperadinyl, 4-(N-piperadinyl)-N-piperadinyl, 4-amino-N-piperadinyl, lower alkoxy, methoxy, ethoxy, lower alkylthio, methylthio, lower haloalkyl, monohalomethyl, dihalomethyl, trihalomethyl, trifluoromethyl, lower haloalkyloxy, monohalomethoxy, dihalomethoxy, trihalomethoxy, trifluoromethoxy, aryl, phenyl, arylalkyl, benzyl, aryloxy, phenoxy, arylalkyloxy, benzyloxy, 5- or 6-membered heteroaryl, lower alkyloxycarbonyl, sulfamoyl and —L—R¹⁴, where L is -(CH₂)₁₋₃- or —O—(CH₂)₁₋₃- and R¹⁴ is a 5- or 6-membered cycloheteroalkyl or N-morpholinyl.

36. The compound of claim 35, wherein R² and R⁶ are each, independently of one another, selected from the group consisting of chloro, bromo, iodo and fluoro.

37. The compound of claim 35, wherein D is O, E is N and F is CH.

38. The compound of claim 35, wherein D is N, E is O and F is CH.

39. The compound according to claim 35, wherein R¹² is —H and R¹⁵ is lower alkyl, arylalkyl, aryl, cycloheteroalkyl, cycloalkyl, or a sugar moiety.

40. The compound according to claim 39, wherein R¹⁵ is piperidyl, pyrrolidinyl, t-butyl, benzyl, cyclobutyl or propyl.

41. The compound according to claim 35, wherein R¹⁶ is lower alkyl or cycloalkyl.

42. The compound according to claim 41, wherein R¹⁶ is t-butyl or adamantane.

43. The compound according to claim 35, wherein R¹⁸ is —H or lower alkyl and R¹⁹ is H or lower alkyl.

44. The compound according to claim 43, wherein R¹⁸ and R¹⁹ are both —H, ethyl or propyl.

45. The compound according to claims 1, wherein s is 0 and G is aryl or substituted aryl.

46. The compound according to claims 45, wherein G is phenyl substituted with one or more groups selected from hydrogen, —F, —Cl, —OMe, —CO₂H, —CO₂t—Bu, —CH₂CO₂Et, methyl —OC(O)CH₃, —OC(O)CH₂N(CH₃)₂, —OC(O)CH₂N(CH₃)Boc, —OC(O)CH₂NH(CH₃), or

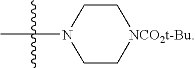

47. The compound according to claims 1, wherein s is 0 and G is substituted arylalkyl.

48. The compound according to claim 47, wherein G is

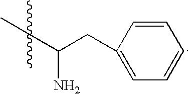

49. The compound according to claims 1, wherein s is 0 and G is heteroaryl.

50. The compound according to claim 49, wherein G is

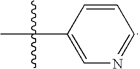

51. The compound according to claims 1, wherein s is 0 and G is cycloheteroalkyl or substituted cycloheteroalkyl.

52. The compound according to claim 51, wherein G is

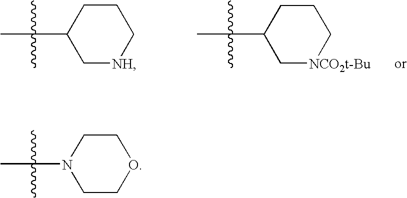

53. The compound of claim 1, wherein when administered to a cell comprising a hepatitis C virion, the compound inhibits HCV replication and/or proliferation and has an IC₅₀ of 10 μM or less, as measured in an in vitro assay.

54. The compound of claim 1 which is selected from the group consisting of those provided in Tables 1 through 13.
